(19) United States
(12) Patent Application Publication
(10) Pub. No.: US 2022/0273824 A1
(43) Pub. Date: Sep. 1, 2022

(54) CELL PENETRATING PEPTIDE FUNCTIONALIZED PERFLUOROCARBON NANOEMULSION COMPOSITIONS AND METHODS FOR IMAGING CELL POPULATIONS

(71) Applicant: THE REGENTS OF THE UNIVERSITY OF CALIFORNIA, Oakland, CA (US)

(72) Inventors: Eric T. Ahrens, Encinitas, CA (US); Dina Hingorani, San Diego, CA (US); Stephen Adams, Poway, CA (US)

(21) Appl. No.: 17/630,286

(22) PCT Filed: Aug. 6, 2020
(86) PCT No.: PCT/US20/45279

§ 371 Date: Jan. 26, 2022

Related U.S. Application Data
(60) Provisional application No. 62/884,111, filed on Aug. 7, 2019.

(51) Int. Cl. A61K 49/00; A61K 49/18; A61B 6/03; A61B 6/00; C07K 14/16

(52) U.S. Cl. CPC A61K 49/0078; A61K 49/0032; A61K 49/1806; A61B 6/032; A61B 6/5235; C07K 14/16

(57) ABSTRACT

This disclosure provides compositions of fluorinated nanoemulsions and associated methods for producing cellular labels for tracking cells by magnetic resonance imaging (MRI), computed tomography (CT), positron emission tomography (PET), and related methods.

### CROSS-REFERENCE TO RELATED APPLICATIONS

This application claims priority to U.S. Provisional Application No. 62/884,111, filed Aug. 7, 2019, which is hereby incorporated by reference in its entirety.

### STATEMENT AS TO RIGHTS TO INVENTIONS MADE UNDER FEDERALLY SPONSORED RESEARCH OR DEVELOPMENT

This invention was made with government support under EB024015 awarded by the National Institutes of Health. The government has certain rights in the invention.

### BACKGROUND OF THE INVENTION

Clinical non-invasive imaging techniques are widely used as diagnostics and to track medical procedures. Magnetic resonance imaging (MRI) is a widely used clinical diagnostic tool because it is non-invasive, allows views into optically opaque subjects, and provides contrast among soft tissues at reasonably high spatial resolution. Conventional MRI mostly focuses on visualizing anatomy and lesions and has no specificity for any particular cell type. The ‘probe’ used by conventional MRI is the ubiquitous proton (¹H) in mobile water molecules. Cells are the fundamental building blocks of any organ system. An exogenous MRI probe or reagent to specifically tag cells is needed to facilitate cell-specific imaging in living subjects. For small animal studies, there are many options available for tracking cells in their native environment, especially using various fluorescent and bioluminescent probes and reporters. However, there remains a great unmet need for cell tracking technologies that have the potential for clinical translation. There are several non-invasive diagnostic imaging modalities that are routinely used in humans including various radioisotope methods, MRI, computed tomography, and ultrasound. Adopting existing diagnostic imaging modalities to visualize cells in the body is a complex problem. Non-invasive imaging of the dynamic trafficking patterns of populations of immune cells can play an important role in elucidating the basic pathogenesis of major diseases such as cancer and autoimmune disorders. Other cell populations, such as tumor or stem cells, can be tracked using MRI to provide insight into metastatic processes, cell engraftment and differentiation, and tissue renewal. Moreover, cells are increasingly being used as therapeutic agents to treat genetic and neurological disorders, as well as chronic conditions such as autoimmunity and cancer. A common need for virtually all cell therapies, particularly at the development stage, is a non-invasive way to detect and quantify the cell biodistribution (e.g, the distribution or location of the cell in the body) following injection. Non-invasive imaging of cell trafficking is capable of providing critical feedback regarding modes of action of the cells, optimal routes of delivery and therapeutic doses for individuals. On the regulatory side, emerging new therapies, such as those using immunotherapeutic and stem cells, are slow to gain regulatory approvals partly because clinical researchers are challenged to verify where the cells go immediately after inoculation and where they migrate to days and weeks later. Cell tracking can potentially provide this information and may help in lowering regulatory approval barriers.

Intimately related to cell trafficking is inflammation and the inflammatory response. Prevalent inflammatory diseases include, for example, arthritis, asthma, atherosclerosis, cancer, diabetes, chronic obstructive pulmonary disease (COPD), inflammatory bowel disease (IBD), infection, multiple sclerosis, and organ transplant rejection. The progression of these diseases can often be slow, and the effectiveness of treatment can be observed only after days, weeks or months. Thus, there is a strong unmet need for inflammation-specific diagnostics, as well as inflammation surrogate biomarkers that permit therapeutic developers to determine efficacy quickly, quantitatively, and in a longitudinal fashion. A related need entails pharmacological safety profiling to detect ‘off target’ inflammatory side effects in pre/clinical drug trials. A non-invasive, image-based biomarker could potentially fill these unmet needs. Vital imaging can accelerate the ‘go/no go’ decision making process at the preclinical and clinical trial stages, and can facilitate smaller, less costly trials by enrolling fewer patients. Imaging can potentially yield quantitative data about inflammation severity and time course in the anatomical context. The highest value imaging biomarker would have broad utility for multiple diseases and be applicable from mouse-to-man, thereby minimizing validation studies.

Fluorine-19 (¹⁹F) ‘tracer’ agents are an emerging approach to intracellularly label cells of interest, either ex vivo or in situ, to enable cell detection via ¹⁹F MRI (Ahrens et al, NMR in Biomed, 2013, 26(7), 860-871; Ahrens and Bulte, Nat Rev Immunol, 201313(10), 755-63). The ¹⁹F label yields positive-signal ‘hot-spot’ images, with no background signal due to negligible fluorine concentration in tissues. Images can be quantified to measure fluorine content in regions of interest yielding a measure of cell numbers at sites of accumulation. Tracer agent compositions have mostly focused on nontoxic perfluorocarbons (PFC). Fluorine-19 is an alternate nucleus that can be imaged using many of today's MRI installations, and this ability is well known in the art.

Often a key limitation of ¹⁹F MRI using various types of probes is sensitivity. Improving the sensitivity of ¹⁹F cell detection could lower the barriers for using these technologies in a much wider range of biomedical applications. Unlike conventional ¹H MRI, where the probe (water) concentration (>100 Molar ¹H) and thus sensitivity is high, ¹⁹F MRI is limited by the total amount and distribution of fluorine atoms introduced into the subject's tissue. In cell tracking and inflammation imaging applications, most often the amount of ¹⁹F in a region of interest is limited by the amount of tracer agent that can be safely internalized into cells of interest. Thus, to improve sensitivity and overall detectability of sparse cell numbers in tissue, one must somehow improve the intrinsic MRI sensitivity of the PFC molecule (or other type of ¹⁹F probe molecule).

Noninvasive methods for tracking cell therapy grafts are an urgent unmet clinical need. With the development of adoptive immunotherapy against cancer, such as using chimeric antigen receptor (CAR) T cell therapy, there is a need to determine the initial biodistribution and survival of the therapeutic cells. Visualizing cell populations in vivo can also provide insights into off-site toxicities and help refine dosing regimens to enhance therapeutic efficacy.

The present invention meets this need.

### BRIEF SUMMARY OF THE INVENTION

In one aspect, provided herein is a nanoemulsion formulation comprising a surfactant, a perfluorocarbon, and a hydrophilic anchor, wherein the perfluorocarbon is conjugated to the hydrophilic anchor via a linker.

In some embodiments, the hydrophilic anchor of the nanoemulsion is at an amount of at least 2% (w/w) of the hydrophilic anchor to the surfactant. In certain embodiments, the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

In some embodiments, the perfluorocarbon of the nanoemulsion comprises perfluoropolyether (PFPE) or perfluoro-15-crown-5-ether (PFCE). In an exemplary embodiment, the perfluorocarbon is conjugated to the hydrophilic anchor via a linker. In some embodiments, the linker is an aliphatic hydrocarbon linker.

In some embodiments, the surfactant of the nanoemulsion comprises a block copolymer of polyethylene and polypropylene glycol.

In certain embodiments, the nanoemulsion further comprises a detectable moiety. In particular embodiments, the detectable moiety is attached to the perfluorocarbon. In certain embodiments, the detectable moiety is a fluorescent moiety.

Provided herein is a nanoemulsion formulation comprising a poloxamer surfactant, a perfluorocarbon, and a TAT peptide of HIV, wherein said perfluorocarbon is conjugated to the hydrophilic anchor via a linker.

Provided herein is a nanoemulsion formulation comprising a poloxamer surfactant, a perfluorocarbon, and a TAT peptide of HIV, wherein said perfluorocarbon is conjugated to the hydrophilic anchor via a linker and said TAT peptide is an amount of at least 2% (w/w) of TAT peptide to surfactant.

Provided herein is a nanoemulsion formulation comprising a poloxamer surfactant, perfluoropolyether (PFPE), and a TAT peptide of HIV, wherein said PFPE is conjugated to the hydrophilic anchor via a linker.

Provided herein is a nanoemulsion formulation comprising a poloxamer surfactant, perfluoro-15-crown-5-ether (PFCE), and a TAT peptide of HIV, wherein said PFCE is conjugated to the hydrophilic anchor via a linker.

Provided herein is a nanoemulsion formulation comprising a poloxamer surfactant, perfluoropolyether (PFPE), and a TAT peptide of HIV, wherein said PFPE is conjugated to the hydrophilic anchor via a linker and said TAT peptide is an amount of at least 2% (w/w) of TAT peptide to surfactant.

Provided herein is a nanoemulsion formulation comprising a poloxamer surfactant, perfluoro-15-crown-5-ether (PFCE), and a TAT peptide of HIV, wherein said PFCE is conjugated to the hydrophilic anchor via a linker and said TAT peptide is an amount of at least 2% (w/w) of TAT peptide to surfactant.

In some embodiments, any of the nanoemulsion formulations further comprises a detectable moiety. In some instances, the detectable moiety is a fluorescent moiety. In some embodiments, the detectable moiety is attached to the perfluorocarbon (e.g., PFPE and PFCE) of the nanoemulsion.

Provided herein is a nanoemulsion formulation comprising a detectable moiety, a poloxamer surfactant, a perfluorocarbon, and a TAT peptide of HIV, wherein said perfluorocarbon is conjugated to the hydrophilic anchor via a linker.

Provided herein is a nanoemulsion formulation comprising a detectable moiety, a poloxamer surfactant, a perfluorocarbon, and a TAT peptide of HIV, wherein said perfluorocarbon is conjugated to the hydrophilic anchor via a linker and said TAT peptide is an amount of at least 2% (w/w) of TAT peptide to surfactant.

Provided herein is a nanoemulsion formulation comprising a detectable moiety, a poloxamer surfactant, perfluoropolyether (PFPE), and a TAT peptide of HIV, wherein said PFPE is conjugated to the hydrophilic anchor via a linker and said TAT peptide is an amount of at least 2% (w/w) of TAT peptide to surfactant.

Provided herein is a nanoemulsion formulation comprising a detectable moiety, a poloxamer surfactant, perfluoro-15-crown-5-ether (PFCE), and a TAT peptide of HIV, wherein said PFCE is conjugated to the hydrophilic anchor via a linker and said TAT peptide is an amount of at least 2% (w/w) of TAT peptide to surfactant.

In some aspect of the invention, provided herein is a non-invasive imaging method comprising: (a) administering to a subject a cellular labelling composition comprising (i) a compound comprising fluorine-19 (¹⁹F) and (ii) a nanoemulsion comprising a surfactant, a perfluorocarbon, and a hydrophilic anchor, wherein the perfluorocarbon is conjugated to the hydrophilic anchor via a linker, wherein the hydrophilic anchor interacts with the one or more cells, and wherein the composition associates with one or more cells; and (b) detecting the association using an imaging modality, wherein the association can include cellular binding and/or cellular uptake.

In some embodiments, the imaging modality is selected from the group consisting of magnetic resonance imaging (MRI), positron emission tomography (PET), single-photon emission coherent tomography (SPECT), ultrasonography (US), and computed tomography (CT).

In some embodiments, the compound comprising fluorine-19 (¹⁹F) comprises a perfluorinated compound.

In some embodiments, the hydrophilic anchor of the nanoemulsion is at an amount of at least 2% (w/w) of the hydrophilic anchor to the surfactant. In some embodiments, the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

In some embodiments, the perfluorocarbon of the nanoemulsion comprises perfluoropolyether (PFPE) or perfluoro-15-crown-5-ether (PFCE). In some embodiments, the perfluorocarbon is conjugated to the hydrophilic anchor via a linker. In some embodiments, the linker is an aliphatic hydrocarbon linker. In some embodiments, the surfactant of the nanoemulsion comprises a block copolymer of polyethylene and polypropylene glycol. In certain embodiments, the nanoemulsion further comprises a detectable moiety. In some embodiments, the detectable moiety is attached to the perfluorocarbon. In certain embodiments, the detectable moiety is a fluorescent moiety.

In some embodiments, the composition outlined allows tracking cells by MRI, wherein the method comprises detecting the cells associated with at least one component of the composition comprising fluorine-19 (¹⁹F). In some embodiments, the one or more cells are immune cells that accumulate at tissue sites as part of an inflammatory response.

In some embodiments, the method is a diagnostic detection method.

In some embodiments, the one or more cells are engineered immune cells that are administered to the subject to treat a disease or condition. In some embodiments, the method is cytotherapy.

In some embodiments, the one or more cells are endogenous cells of the subject. In some cases, the one or more cells are autologous to the subject. In other cases, the one or more cells are allogeneic to the subject.

In some embodiments, the one or more cells of the method are selected from the group consisting of T cells, B cells, macrophages, natural killer (NK) cells, dendritic cells (DCs), stem cells, progenitor cells, and cancer cells. In some embodiments, the one or more cells comprise engineered cells.

In some embodiments, the one or more cells are engineered chimeric antigen receptor (CAR) T cells that are administered to a subject to treat cancer.

In some embodiments, the compound comprising fluorine-19 (¹⁹F) is a dual-mode agent and is capable of being detected by more than one imaging modality. In certain embodiments, the compound comprising fluorine-19 (¹⁹F) is a dual-mode agent and is capable of being detected by two or more imaging modalities.

In another aspect, provided herein is an in vivo imaging method comprising: (a) ex vivo labeling cells with a cellular labelling composition comprising (i) a compound comprising fluorine-19 (¹⁹F) and (ii) a nanoemulsion comprising a surfactant, a perfluorocarbon, and a hydrophilic anchor, wherein the perfluorocarbon is conjugated to the hydrophilic anchor via a linker, under such conditions that the composition is internalized by the cells; (b) administering the labeled cells to a subject; (c) detecting the labeled cells in the subject using an imaging modality; and (d) assaying for the degree of cell accumulation in one or more tissues in the subject.

In some embodiments, the assaying step of the method comprises quantitating an average total intracellular probe mass at one or more sites of accumulation of the labeled cells.

In some embodiments, the cells of the method are autologous cells. In particular embodiments, the cells are allogeneic cells.

In some embodiments, the imaging modality is selected from the group consisting of magnetic resonance imaging (MRI), positron emission tomography (PET), single-photon emission coherent tomography (SPECT), ultrasonography (US), and computed tomography (CT). In certain embodiments, the imaging modality is magnetic resonance imaging (MRI).

In some embodiments, the cells are selected from the group consisting of T cells, B cells, macrophages, natural killer (NK) cells, dendritic cells (DCs), stem cells, progenitor cells, and cancer cells. In some embodiments, the cells are engineered cells.

In some embodiments, the compound comprising fluorine-19 (¹⁹F) comprises a perfluorinated compound.

In some embodiments, the hydrophilic anchor of the nanoemulsion is at an amount of at least 2% (w/w) of the hydrophilic anchor to the surfactant. In certain embodiments, the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

In some embodiments, the perfluorocarbon of the nanoemulsion comprises perfluoropolyether (PFPE) or perfluoro-15-crown-5-ether (PFCE). In an exemplary embodiment, the perfluorocarbon is conjugated to the hydrophilic anchor via a linker. In some embodiments, the linker is an aliphatic hydrocarbon linker.

In some embodiments, the surfactant of the nanoemulsion comprises a block copolymer of polyethylene and polypropylene glycol.

In certain embodiments, the nanoemulsion further comprises a detectable moiety. In particular embodiments, the detectable moiety is attached to the perfluorocarbon. In certain embodiments, the detectable moiety is a fluorescent moiety.

In one aspect, provided herein is a pharmaceutical and/or diagnostic composition comprising a compound comprising fluorine-19 (¹⁹F) and a nanoemulsion formulation comprising a surfactant, a perfluorocarbon, and a hydrophilic anchor, wherein the perfluorocarbon is conjugated to the hydrophilic anchor via a linker, wherein the composition associates with one or more cells and wherein the association is capable of being detected using an imaging modality.

In some embodiments, the compound of the pharmaceutical and/or diagnostic composition comprises fluorine-19 (¹⁹F) comprising a perfluorinated compound.

In some embodiments, the hydrophilic anchor of the pharmaceutical and/or diagnostic composition interacts with the one or more cells. In certain embodiments, the hydrophilic anchor is at an amount of at least 2% (w/w) of the hydrophilic anchor to the surfactant. In some embodiments, the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

In some embodiments, the perfluorocarbon of the pharmaceutical and/or diagnostic composition comprises perfluoropolyether (PFPE) or perfluoro-15-crown-5-ether (PFCE). In some embodiments, the perfluorocarbon is conjugated to the hydrophilic anchor via a linker. In some embodiments, the linker is an aliphatic hydrocarbon linker. In some embodiments, the surfactant comprises a block copolymer of polyethylene and polypropylene glycol. In certain embodiments, the nanoemulsion further comprises a detectable moiety. In some embodiments, the detectable moiety is attached to the perfluorocarbon. In certain embodiments, the detectable moiety is a fluorescent moiety.

In some embodiments, the pharmaceutical and/or diagnostic composition comprises at least two compounds comprising fluorine-19 (¹⁹F), wherein the at least two compounds provide at least two distinct signatures when detected using an imaging modality capable of individual detection.

In some embodiments, the imaging modality is selected from the group consisting of magnetic resonance imaging (MRI), positron emission tomography (PET), single-photon emission coherent tomography (SPECT), ultrasonography (US), and computed tomography (CT). In some embodiments, the distinct signatures correspond to multiple cell types, the same cell type at different time points, or multiple molecular epitopes within a subject.

In some embodiments, the compound comprising fluorine-19 (¹⁹F) is a theranostic agent. In certain embodiments, the theranostic agent functions as both a therapeutic agent and an imaging probe. In some embodiments, the theranostic agent allows for visualizing the accurate delivery and dose of the therapy within the subject.

Detailed descriptions of PFC containing nanoemulsions are disclosed in Hingorani et al., Magn Reson Med, 2020, 83:974-987 (published Oct. 21, 2019), U.S. Pat. No. 9,352,057, PCT published application WO2017/147212, and U.S. Provisional Application No. 62/777,008, the disclosures including the examples, figures, and figure legends are incorporated by reference herein in their entirety.

### BRIEF DESCRIPTION OF THE DRAWINGS

FIGS. 1A-1B. Synthesis of TAT functionalized perfluorocarbon nanoemulsions. Panel (A) displays the synthesis of TAT conjugates with fluorous anchors, TATP and TATA and poloxamer surfactant formulated nanoemulsions. Panel (B) shows an exemplary scheme for TAT-phospholipid anchor conjugation for EYP surfactant formulated nanoemulsions.

FIGS. 2A-2E. T cell labeling with TATA-F68-PFC and TATP-F68-PFC nanoemulsions. The TAT anchor stoichiometry is optimized by measuring uptake (A) and viability (B) in Jurkat cells while varying the percent by weight of TAT in Pluronic surfactant PFC nanoemulsion, namely TATP-F68-PFC (black bars) and TATA-F68-PFC (grey bars). No significant differences are noted. Cell uptake (C) and viability (D) for varying dosages (in mg/mL) of 10% w/w TATP-F68-PFC and TATA-F68-PFC after 18 hour incubation are shown (p<0.01, uptake TATP-F68-PFC and TATA-F68-PFC. No significant differences are noted for viability) CAR T cells labeled using the same conditions exhibit an 8.2-fold uptake improvement compared to control F68-PFC labeled cells at a dose of 15 mg/ml (dashed bars, * indicates p<0.001, (E). The viability of labeled CAR T cells is displayed above the bar graph. Uptake was measured from ¹⁹F NMR spectra of cell pellets, and viability was measured by the Trypan blue assay and direct cell counts.

FIGS. 3A-3C. Jurkat T cell labeling with lipid-TAT-PFC nanoemulsion. The TAT anchor stoichiometry is optimized by measuring uptake (A) in cells while varying the percent by molarity of TAT in phospholipid surfactant nanoemulsions using two different methods of preparation including post-insertion (dark grey) and direct insertion (light grey) of TAT conjugate. There is no statistical difference between the two insertion methods. The cell uptake (B) and viability (C) with varying dosage of 0.1 mol % lipid-TAT-PFC after 18 hour incubation are displayed. No significant viability impairment is noted.

FIGS. 4A-4L. Microscopy of CAR T cells labeled with TAT-F68-PFC nanoemulsions. Confocal microscopy images of untreated CAR T cells are displayed in (A), and CAR T cells labeled with (15 mg/mL) of Cy5-TATP-F68-PFC nanoemulsions (red) are shown in (B). Data show intracellular localization of Cy5-TATP-F68-PFC emulsion, where Hoechst dye (blue) stains nuclei and Alexa488 anti-human CD3 antibody (green) delineates cell membrane. Electron microscopy of untreated CAR T cells is shown in (C) and magnified in (D). CAR T cells labeled with TATP-F68-PFC (E-H) show numerous bright ˜100 nm nanoemulsion droplets (E, magnified in F, arrows) and occasional ˜1 μm coalesced droplets (G, magnified in H, arrows). CAR T cells labeled with TATA-F68-PFC (I-L) show similar nanoemulsion droplets as with TATP-F68-PFC nanoemulsion. Large coalesced droplets (I, inset J) as well as numerous smaller droplets (K, inset L) are found in the cytoplasm.

FIGS. 5A-5F. Phenotype of CAR T cells labeled with TAT-F68-PFC nanoemulsions. Scatter plots confirm pure population of CAR T cells (CD3) (A-C). CD3 expression is unaltered after labeling with TATP-F68-PFC (A) or TATA-F68-PFC (B) nanoemulsions compared to unlabeled cells (C). Flow analysis for expression of CD4/CD8 shows a ˜90/10 ratio of CD4+ to CD8+ positive cells (D-F). CAR T cells labeled with TATP-F68-PFC (D) or TATA-F68-PFC (E) ex vivo exhibit comparable phenotype to unlabeled cells (F). FSC-A indicates forward scatter, FITC stands for fluorescein isothiocyanate, and PE/Cy5 is phycoerythrin-cyanine 5.

FIGS. 6A-6F. In vivo ¹⁹F MRI signal enhancement in TATP-F68-PFC labeled human CAR T cells. Panel (A) displays composite ¹⁹F (hot-iron) and ¹H (grayscale) contiguous slices of a mouse with bilateral gliomas in the flanks, where the left and right tumor (LT, RT) each received 1×10⁷ CAR T cells labeled with either F68-PFC (control) or TATP-F68-PFC nanoemulsions, respectively. An external capillary reference (REF) is also shown in the field of view consisting of 1.20 dilution of F68-PFC in agarose. Panel (B) displays a three-dimensional rendering of the MRI data shown in (A). MRI data were acquired at 11.7 T using RARE sequences for ¹⁹F and ¹H. A histogram of the ¹⁹F signal-to-noise ratio for each image voxel in the tumors is displayed in (C) and shows sensitivity improvement of the TATP-containing nanoemulsion compared to control. Comparison of apparent ¹⁹F atoms per tumor, as measured in vivo for N=4 mice, is displayed in (D) showing ˜8-fold sensitivity enhancement (* indicates p<0.001) for TAT-F68-PFC nanoemulsions compared to control. To verify intratumoral delivery of CAR T cells, two days after CAR T cell injection, tumors were excised and fixed for extremely high-resolution MRI. Panels (E and F) show composite ¹⁹F/¹H three-dimensional renderings of intratumoral CAR T cells labeled with control and TAT-F68-PFC nanoemulsions, respectively. Data in (E and F) were acquired at 9.4 T at 100 μm isotropic resolution using RARE (¹⁹F) and spin-echo (¹H) imaging sequences.

FIG. 7. Synthesis scheme of F68-TAT co-surfactant. F68 is functionalized with a maleimide group to enable addition of the TAT peptide with a terminal cysteine (Cys-TAT).

FIGS. 8A-8D. Size stability of TAT-F68-PFC nanoemulsions. The effect of % TAT incorporation on size (A) and polydispersity index (PDI, B) of nanoemulsions is shown. The nanoemulsion size (C) and PDI (D) of nanoemulsions over time while stored at 4° C. is displayed.

FIGS. 9A-9B. Optimization of lipid-TAT-PFC incubation time in Jurkat cells. Incubation times of 2, 4 and 18 hours are tested as shown in (A), and the highest uptake is observed at 18 hours. Jurkat cell viability is not altered by labeling for different durations (B).

FIG. 10. Cy5-TATA, P-F68-PFC synthesis scheme. Scheme shows synthesis of fluorescently labeled co-surfactants 8 and 9 consisting of Cy5 dye attached to the respective fluorous anchors 6 and 7 for incorporation into TATP-F68-PFC and TATA-F68-PFC nanoemulsions.

FIGS. 11A-11C. Localization impact of incorporation of fluorescent dye into surfactant layer during nanoemulsion preparation. Panel (A) displays ¹⁹F uptake for cells treated with nanoemulsions prepared with and without anchored Cy5 at 10 mg/mL and 20 mg/mL doses; no significant differences are observed. Additionally, CAR T cell viability is not affected as shown in (B). Panel (C) shows intracellular localization of the nanoemulsion (Cy5 in red) in CAR T cells via confocal microscopy. Hoechst dye (nuclei, blue) and Alexa488 dye (cell membrane, green) is used to delineate cell structures.

FIG. 12 Fluorescent dye conjugate nanoemulsions without TAT do not get internalized into CAR T cells. Panels show that dye compounds 8 and 9 do not induce non-specific internalization into live cells. Hoechst dye (nuclei, blue) and Alexa488 dye (cell membrane, green) are used to delineate the cells.

FIG. 13. CAR T cell killing assay in vitro. Co-incubation of human U87-EGFRvIII-Luc glioma cells with TATP-F68-PFC-labeled or unlabeled CAR T cells, or untransduced T cells results in significant cell death at 12 and 24 h. CAR T cells exhibit significant tumor killing ability (˜98%) compared to untransduced T cells (˜60%). Killing efficacy is unaltered by nanoemulsion labeling of the cells.

FIG. 14. Ex vivo 3D microimaging of excised glioma tumors harboring PFC labeled CAR T cells. Contiguous images show overlays of ¹⁹F (pseudo-color) and ¹H (grayscale) slices of right tumor receiving an intratumoral injection of 10⁷ TATP-F68-PFC labeled CAR T cells (A), and the left tumor with the same number of F68-PFC labeled CAR T cells (B).

FIG. 15. Examples of commonly used functional groups for covalent coupling.

FIG. 16. Examples of fluorous anchor moieties of the co-surfactant for interaction with the perfluorocarbon oil.

FIG. 17. Examples of linker molecule to separate chemically dissimilar molecules in the co-surfactant.

FIG. 18. Examples of reactive functional groups.

### DETAILED DESCRIPTION OF THE INVENTION

The invention describes compositions and methods for the formulation of perfluorocarbon (PFC) based emulsions (PFC emulsions) that are functionalized with a peptide, or other moiety, on the emulsion droplet's surface as a component of the surfactant layer. Outlined herein is an agent comprising a colloidal medium comprising perfluorocarbons contained within a surfactant and co-surfactant coating. The novel co-surfactant increases uptake by cells resulting, for example, in enhanced imaging sensitivity and reduces scanning time. This agent is stable, non-toxic and useful, for example, for non-invasive in vivo cell tracking for visualization of engineered immune cell homing in vivo.

In one aspect, the invention provides a composition or formulation of a co-surfactant comprising a fluorous anchor moiety, a linker to mimic the chemical nature of the surfactant, and a hydrophilic anchor such as a cell penetrating peptide.

In some embodiments, the components comprising the co-surfactant maybe connected to each other in a linear of branched synthesis scheme using any of the commonly known chemical coupling chemistries that are prevalent in the literature. The same covalent bond between two functional groups can be formed by various reagents (synthetic, biological, electromagnetic spectrum or by physical force) and reaction types. In addition to cysteine-maleimide chemistry, amide formation through activated carboxylic acids or using coupling reagent (carbodiimide etc), click reaction with propargyl/azide containing non-natural amino acid or heterobifunctional linker and any other standard methods known in the field for conjugating peptides/proteins to small molecules.

Such an agent can be used in clinical non-invasive imaging methods, particularly magnetic resonance imaging (MRI), to visualize cells and cells targets in the body. The agent is useful as an imaging probe for fluorine-19 (¹⁹F) magnetic resonance imaging (MRI) and nuclear imaging (e.g., PET and SPECT) of therapeutic cell products infused in the body and for diagnostic imaging. For instance, cells (e.g., target cells) labelled with the fluorine-19 containing compositions provided herein can be visualized (imaged, tracked, tracked, and the like) in a subject, e.g., a human subject, and quantitated.

The compositions described herein are useful for MRI as they can provide a single sharp resonance, provide desireable signal intensity and signal-to-noise ratio (SNR) efficiency, eliminate any chemical shift artifact, maximize the SNR, are thermodynamically stable, and allow clear identification of the perfluorinated compound.

Other embodiments of the invention are metalated perfluorinated probes that can be detected by positron emission tomography (PET), single-photon emission coherent tomography (SPECT), ultrasonography (US), or computed tomography (CT), all of which are commonly used medical imaging modalities. The invention provides novel uses for these imaging modalities by providing a means to detect inflammatory cells and track cytotherapies non-invasively. Also, so called “dual-mode” agents are envisioned, which can be detected by more than one imaging modality (e.g., MRI-PET), thereby maximizing the utility of new generations of clinical imaging apparatus that integrate two (or more) detection modalities.

Some applications include the diagnostic detection of immune cells that accumulate at tissue sites as part of an inflammatory response and cells that are grafted into the body in order to treat a disease or condition, i.e., cytotherapy. Cells can be endogenous cells in the body, for example, various immune cells (T cells, B cells, macrophages, NK cells, DCs, etc.), various stem cells and progenitor cells, cancer cells, as well as engineered cells, which are often used in cytotherapy in its various forms. Non-invasive imaging of immune cells in the body is useful because it can aid in the diagnosis and monitoring of inflammation. In the field of cytotherapy, the ability to image the cell graft provides valuable feedback about the persistence of the graft, potential cell migration, and improves safety surveillance. Many experimental cell therapies that are in clinical trials, e.g., stem cells and immunotherapeutic cells, could benefit from the use of this technology.

Additionally, ex vivo or in vivo targeted imaging and theranostic agents are described using the molecular platform that provide imaging of cells, tissues, and/or lesions having selected and prevalent molecular epitopes. For example targeting moieties can include antibodies (or fragments thereof), peptides, arginine-rich domains, cationic lipids, aptamers, etc.

The compositions of the invention can be used for targeted drug delivery and theranostic applications. Such theranostic agents may serve both as a therapeutic (or drug delivery vehicle) agent and an imaging probe (or diagnostic agent) that can help visualize the accurate delivery and dose of the therapy within the body. The pharmaceutical and/or diagnostic composition disclosed herein can be administered to a subject, the delivery of the composition (or cells labelled with the composition), and the dose/amount of the composition can be detected, monitored, tracked, and/or measured in the subject.

The invention also describes synthetic schemes and methods for the chemical attachment of peptides to the surface of the PFC emulsions. The peptide attachment imparts new functionalities to the PFC emulsions. In some embodiments, peptide-PFC emulsions of the present invention can be used for boosting emulsion uptake into cells labeled ex vivo for use in in vivo imaging after grafting to the subject. Described herein is an example of how a peptide (TAT) can be used to dramatically increase cellular uptake (>8-fold) of an imaging agent in therapeutic T cells; importantly, the imaging sensitivity for ¹⁹F MRI detection of labeled cells scales proportionally to cellular uptake of labeling probe enabling more reliable detection and the detection of more dilute cell deposits in vivo.

Provided herein is a novel approach for boosting sensitivity for cell imaging is via rational chemical design of the imaging probe.

The invention also describes novel methods to assay the degree of cell labeling with the imaging probe, for example, as represented by the average total intracellular probe mass following labeling. Methods for quantitating labeled cells include methods known by those skilled in the art and used in MRI, PET, SPECT, US, and CT imaging.

In some embodiments, the compositions or formulations includes a first compound comprising ¹⁹F have a first ¹⁹F spectral frequency and a second compound comprising ¹⁹F have a second ¹⁹F spectral frequency that is different than the first ¹⁹F spectral frequency. In some instances, the first compound includes a first metal ion and the second compound includes a second metal ion, such that the first and second metal ions are different. Detailed descriptions of fluorous metal chelates are disclosed in U.S. Pat. No. 9,352,057, PCT Publication No. WO2017/147212 filed Feb. 22, 2017, and U.S. Provisional Application Nos. 62/298,430 filed Feb. 22, 2016 and 62/777,008 filed Dec. 7, 2018, the disclosures including the examples, figures, and figure legends are incorporated by reference herein in their entirety. The first compound and the second compound can provide two separate, different spectral frequencies (i.e., two distinct imaging signatures) when detected simultaneously. In other cases, the first and second compounds are detected sequentially. The compounds can be detected using one imaging modality, e.g., MRI. In some cases, the compounds are detected using two different imaging modalities, such as, but not limited to, MRI and PET, MRI and SPECT, and PET and SPECT.

In some instances, the first ¹⁹F-containing compound labels a first cell type, and the second ¹⁹F-containing compound labels a second cell type. In certain cases, the first ¹⁹F-containing compound labels a cell type at a first time point, and the second ¹⁹F-containing compound labels the same cell type at a second time point (i.e., a later time point). In other cases, the first ¹⁹F-containing compound comprises a first targeting moiety that specifically binds to a first cell type, and the the second ¹⁹F-containing compound comprises a second targeting moiety that specifically binds to a second cell type. The first and second cell types can be introduced into the subject. Optionally, the first and second cell types can be two different endogenous cell types located in the subject. In some embodiments, two, three or four different cell types can be introduced.

The present invention provides peptide-PFC nanoemulsions formulated entirely from synthetic components. Prior art nanoemulsions employ phospholipid surfactants to form nanoemulsion that mimic the membranes of live cells and impart biocompatibility. However, phospholipid-formulated emulsions are prone to instability under storage conditions due to oxidation-mediated changes to the lipid that limits shelf-life, especially if metal ions are present, and lipid oxidation by-products may lead to cytotoxicity upon cell contact. Additionally, the formulation of phospholipid-based nanoemulsions requires a time-consuming multi-step chemical process. Outlined herein are novel and improved PFC nanoemulsions comprising cell penetrating peptides conjugated with synthetic polymeric co-surfactants.

### DETAILED DESCRIPTION OF EMBODIMENTS

In one aspect of the invention, provided herein are compositions or formulations and methods for the formulation of perfluorocarbon (PFC) based emulsions that are functionalized with a peptide, or other moiety, on the emulsion droplet's surface as a component of the surfactant layer. Provided herein are methods for ex vivo cell labeling using perfluorocarbon (PFC) nanoemulsions comprising a cell-penetrating peptide (CPP). The peptide-PFC nanoemulsions can be paired with ¹⁹F MRI detection and used as a non-invasive approach for cell product detection in vivo. The peptide can be conjugated to the co-surfactant of the nanoemulsions.

Also, provided herein are PFC nanoemulsion imaging probes displaying cell-penetrating peptides. In some embodiments, the PFC nanoemulsion imaging probes are used for non-invasively imaging cell populations in a subject. In some embodiments, the cell-penetrating peptide is a transactivating transcription sequence (TAT) of the human immunodeficiency virus.

In some embodiments, the PFC of the nanoemulsion comprises perfluoropolyether (PFPE, a perfluorinated polyethylene glycol). In some embodiments, the PFC comprises perfluoro-15-crown-5-ether (PFCE). In some embodiments, the nanoemulsion comprises a poloxamer surfactant and a CPP.

In some embodiments, the co-surfactant of the nanoemulsion comprises a phospholipid surfactant and a CPP. In some embodiments, the peptide-PFC nanoemulsion further comprise a detectable agent. In some instances, the detectable agent is a fluorescent dye.

In some embodiments with the poloxamer surfactant, the CPP is conjugated with a terminal cysteine directly to the poloxamer that has been functionalized with a maleimide group. In some embodiments, the poloxamer is Pluronic™ F68.

In some embodiments, the CPP with a terminal cysteine is conjugated to one or two small linear fluorous molecule anchors via a short aliphatic hydrocarbon linker that comprises a maleimide group, wherein the fluorous molecule anchors comprise a perfluoroheptyl group or a perfluoroPEG group.

In some instances, the peptide-PFC nanoemulsion composition comprising a phospholipid surfactant is made by a direct insertion of the peptide conjugate method. In other instances, the peptide-PFC nanoemulsion composition comprising a phospholipid surfactant is made by a post-insertion of the peptide conjugate method.

In some embodiments, the invention provides an agent comprising a colloidal medium comprising perfluorocarbons contained within a surfactant and co-surfactant coating. The novel co-surfactant increases uptake by cells resulting, for example, in enhanced imaging sensitivity and reduces scanning time. This agent is stable, non-toxic and useful. The agent can be used for non-invasive in vivo cell tracking for visualization of engineered immune cell homing in vivo.

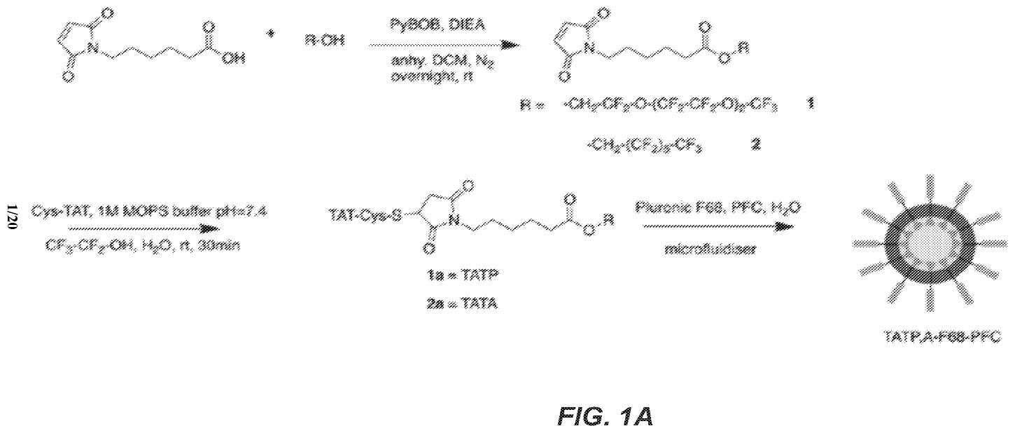

In some embodiments, the co-surfactant comprises a fluorous anchor moiety, a linker, and a hydrophilic anchor. In some embodiments, the hydrophilic anchor comprises a cell penetrating peptide. Exemplary examples of a peptide-PFC nanoemulsion of the present invention are shown in FIG. 1A and FIG. 1B.

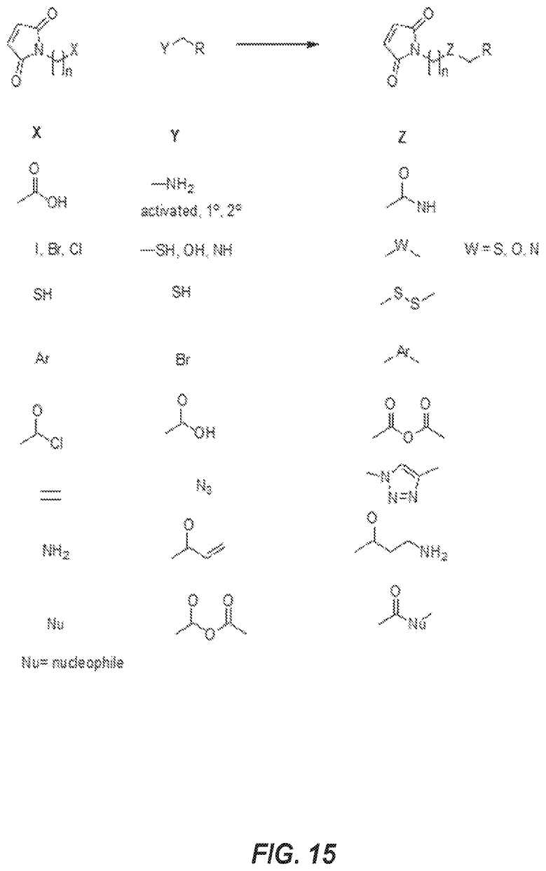

In some embodiments, the three key components of the co-surfactant are connected to each other in a linear of branched synthesis scheme using any of the commonly known chemical coupling chemistries that are prevalent in the literature. In some instances, the same covalent bond between two functional groups are formed by various reagents (synthetic, biological, electromagnetic spectrum or by physical force) and reaction types. Non-limiting examples of chemical coupling chemistries include cysteine-maleimide chemistry, amide formation through activated carboxylic acids or using coupling reagent (carbodiimide etc), click reaction with propargyl/azide containing non-natural amino acid or heterobifunctional linker and any other standard methods known in the field for conjugating peptides/proteins to small molecules. Exemplary examples of functional groups for covalent coupling are depicted in FIG. 15.

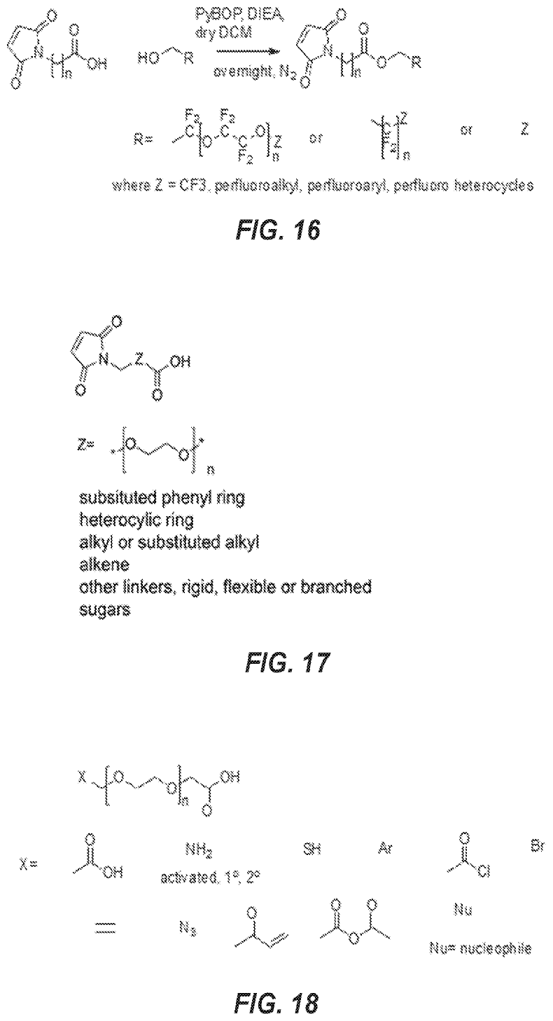

In some embodiments, the fluorous anchor moiety is linear or branched for maintaining optimal stability of the nanoemulsion. Exemplary examples of fluorous anchor moieties of the co-surfactant for interaction with the perfluorocarbon oil are shown in FIG. 16.

In some embodiments, the linker separating the hydrophobic and lipophobic fluorous anchor and the hydrophilic anchor (e.g., the cell interacting sequence) has the ability to mimic the surfactant chosen for encapsulation of the perfluorocarbon oil. Non-limiting examples of a linker are depicted in FIG. 17. The criteria for selection of the linker involves the ability to mimic the surfactant chosen for encapsulation of the perfluorocarbon oil. The selection of the reactive functional group on either end of the linker are modifiable (such as but not limited to those depicted in FIG. 18) based on the selected coupling chemistry include those depicted in FIG. 15.

In some embodiments, the hydrophilic anchor (or cell interacting moiety) comprises any one selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

In some embodiments, the co-surfactant is purified by standard purification techniques of high pressure liquid chromatography (HPLC) and normal phase chromatography. In some embodiments, the purity and mass of the co-surfactant is determined by liquid chromatography mass spectroscopy (LC-MS) prior to use.

In some embodiments, the co-surfactant is stored in aliquoted amounts as a dry powder after lyophilization and refrigerated at −20° C. for subsequent use.

In one aspect, the invention describes synthetic schemes and methods for the chemical attachment of peptides to the surface of PFC emulsions. Peptide attachment imparts novel functionalities to PFC emulsions. For example, peptide-PFC emulsion can be used for boosting emulsion uptake into cells labeled ex vivo for use in in vivo imaging after grafting to the subject. The example below illustrates the use of a peptide (TAT) to dramatically increase cellular uptake (>8-fold) of an imaging agent in therapeutic T cells; importantly, the imaging sensitivity for ¹⁹F MRI detection of labeled cells scales proportionally to cellular uptake of labeling probe enabling more reliable detection and the detection of more dilute cell deposits in vivo.

In some embodiments, the peptide-PFC nanoemulsion labels immune cells, such as but not limited to, lymphocytes, primary T cells, primary human chimeric antigen receptor (CAR) T cells, primary B cells, primary NK cells, and the like. The cells are labeled by the PFC nanoemulsion by incubating the cells with the PFC nanoemulsion under specific conditions. In some embodiments, the cells are incubated with the PFC nanoemulsion for about 10-20 hours at about 37° C. In particular embodiments, the cells are incubated with the PFC nanoemulsion for about 10-20 hours at about 37° C. and 5% CO₂.

In some embodiments, the peptide-PFC nanoemulsion described herein are endocytosed by non-phagocytic cells. In some embodiments, the peptide-PFC nanoemulsions results in a 4- to 8-fold increase in cell loading compared to a corresponding nanoemulsion.

In some embodiments, the peptide-PFC nanoemulsion comprises at least 2% w/w CPP to surfactant. In certain embodiments, the peptide-PFC nanoemulsion comprises at least 10% w/w CPP to surfactant. In particular embodiments, the peptide-PFC nanoemulsion comprises at least 15% w/w CPP to surfactant.

In some embodiments, the peptide-PFC nanoemulsions are stable at 4° C. for at least one month. In some embodiments, the peptide-PFC nanoemulsions are stable at 4° C. for at least two month or more In some embodiments, the peptide-PFC nanoemulsions are stable at 4° C. for at least 6 month or more.

### Uses of Peptide-PFC Nanoemulsions

Peptides can be used for targeting of PFC, either to cells ex vivo prior to administration, or in situ in the body following infusion. Cell-specific targeting in culture, ex vivo, is appropriate for adaptive cell therapies and regenerative medicine using stem/progenitor cells. In the case of in situ targeting of peptide-PFC nanoemulsions following infusion, the addition of the peptide enables targeting disease for more precise diagnosis using non-invasive imaging, for example to detect thrombosis, atherosclerosis, cancer, etc. A range of peptides are known in the art that can target lesions, specific cell phenotypes, and molecules of interest in vivo for diagnostic and therapeutic purposes.

In some embodiments, the peptide-PFC emulsions are used as targeted drug delivery vehicles. In certain embodiments, the peptide-PFC emulsions are used for delivery of anti-inflammatory agents, such as various steroids and nonsteroidal anti-inflammatory agents, to treat pain and other inflammatory conditions. In certain embodiments, the peptide-PFC emulsions are used as vehicles for interleukins, chemokines, immunomodulators, anti-cancer drugs and the like. Methods for association of a drug to a PFC emulsion are known in the art. An exemplary method includes, but is not limited to, the inclusion of hydrocarbon layer between PFC and surfactant-peptide to absorb lipophobic test articles.

The compositions of the present invention can be combined with other emulsion chemical and formulation modifications. In some embodiments, the addition of fluorous metal chelates dissolved in the fluorous phase of the emulsion imparts additional functionality to the peptide-PFC nanoemulsion. In some instances, metal chelates in PFC sequesters and tightly binds metal ions from the emulsion buffer into the fluorous phase (see, e.g., Kislukhin et al., Nat Mater, 2016, 15(6), 662-668). These metal ions can consist of radioactive isotopes used for nuclear imaging, such as 89Zr, 68Ga, etc., which are used for positron emission tomography (PET) and single photon emission computed tomography (SPECT). In some cases, the peptide-PFC emulsions are used as a probe for PET/SPECT detection when a radioactive metal ion is bound. Detailed descriptions of fluorous metal chelates useful for nanoemulsions are disclosed in U.S. Pat. No. 9,352,057, PCT Publication No. WO2017/147212 filed Feb. 22, 2017, and U.S. Provisional Application Nos. 62/298,430 filed Feb. 22, 2016 and 62/777,008 filed Dec. 7, 2018, the disclosures including the examples, figures, and figure legends are incorporated by reference herein in their entirety.

In another aspect, provided herein are compositions of peptide-PFC emulsion comprising a therapeutic drug. Such peptide-PFC nanoemulsions may be used as a theranostic probe, wherein drug delivery is detected via non-invasive imaging methods, such as but not limited to, MRI and PET/SPECT.

The compounds, compositions, and methods described herein can be used to track or trace cells by an imaging method, such as MRI, by detecting the cells associated (labeled) with the fluorine-19 containing compound or composition.

In some embodiments, the compounds, compositions, and methods are used to diagnose a disease by detecting or tracking the labeled cells, e.g., labeled immune cells. In some cases, the compounds and compositions can be administered to a subject to label a specific cell type. In other cases, cells of interest are labeled with the compounds and compositions in vitro, the labeled cells are administered to a subject, and the cells are detected using an imaging modality, e.g., MRI, PET, SPECT, CT, and ultrasound. The cells can be engineered cells, such as cells that express recombinant DNA encoding one or more recombinant proteins. In some cases, the recombinant protein is a targeting moiety, such as antibodies and fragments thereof, peptides, arginine-rich domains, cationic lipids, and aptamers.

The compounds, compositions, and methods described herein can be used for cytotherapy, e.g., cell-based treatment of a disease or condition. Cytotherapy includes introducing, administering, or grafting therapeutic cells into a tissue in order to treat a disease or condition. In other embodiments, the compounds and compositions are used to treat a disease or condition by administering or grafting cells labeled with the fluorine-19 containing compound or composition to a subject in need thereof. The labeled cells can be autologous or allogeneic cells. The cells can also be engineered cells, such as cells that express recombinant DNA encoding one or more recombinant proteins. In some cases, the recombinant protein is a therapeutic protein, e.g., antibody or a fragment thereof. The recombinant protein can be a targeting moiety, such as antibodies and fragments thereof, peptides, arginine-rich domains, cationic lipids, and aptamers.

The compounds and compositions can be an imaging probe that can be used for in vivo applications (e.g., diagnostic detection methods, cytotherapeutic methods, and the like). For instance, cells labeled with the compounds and compositions can be monitored after administration to a subject to determine the biodistribution of the labeled cells or uptake of the labeled cells in the subject.

### PFC Nanoemulsions Comprising a CPP and a Poloxamer Surfactant

Provided herein is a method of synthesizing peptide-PFC nanoemulsion comprising a poloxamer surfactant. In some embodiments, the peptide-PFC nanoemulsion comprises a hydrophilic anchor, a perfluorocarbon and a poloxamer surfactant. In some embodiments, the perfluorocarbon is conjugated to the hydrophilic anchor via a linker. In some embodiments, the peptide-PFC nanoemulsion comprises a cell penetrating peptide, a perfluorocarbon and a poloxamer surfactant. In some embodiments, the peptide-PFC nanoemulsion comprises a transactivating transcription peptide, a perfluorocarbon and a poloxamer surfactant. In some embodiments, the peptide-PFC nanoemulsion comprises a transactivating transcription peptide of a virus (such as but not limited to HIV), a perfluorocarbon and a poloxamer surfactant. In some instances, the perfluorocarbon includes perfluoropolyether (PFPE) or perfluoro-15-crown-5-ether (PFCE). In some embodiments, the perfluorocarbon in any of the nanoemulsions is conjugated to the hydrophilic anchor (e.g., a cell penetrating peptide) via a linker (such as but not limited to aliphatic hydrocarbon linker). In some embodiments, the surfactant comprises a block copolymer of polyethylene and polypropylene glycol. In some embodiments, the surfactant is a Poloxamer, such as Pluronic® F68. In some embodiments, the hydrophilic anchor is linked to the surfactant via an aliphatic hydrocarbon linker. For instance, the hydrophilic anchor is not directly conjugated to the poloxamer surfactant. In some embodiments, the nanoemulsion comprises a detectable moiety.

Provided herein are PFC nanoemulsions comprising TAT peptides (e.g., cell penetrating peptides), poloxamer surfactants (e.g., Pluronic® F68), and F-dense perfluorocarbon molecules (e.g., PFPE and PFCE). Exemplary TAT peptides can have an amino acid sequence as set forth in, for example, Uniprot No. P04608 and NCBI Ref. Seq. No. NP_057853.1. In some embodiments, a TAT peptide with a terminal cysteine is conjugated to a fluorous molecule anchor via a short hydrocarbon linker bearing a maleimide group that is synthesized from the corresponding alcohols and PyBOP as a conjugation agent. In some embodiments, the fluorous molecule anchor comprises a perfluoroheptyl (TATA) or a short perfluoroPEG group (TATP). The anchors are incorporated with variable percentages by weight (% wt). Useful TAT doped PFC nanoemulsions include TATA-F68-PFC and TATP-F68-PFC, as provided in the figures including FIG. 1A. In some embodiments, the PFC nanoemulsion is referred to as a TATA-F68-PFC nanoemulsion. In some embodiments, the PFC nanoemulsion is referred to as a TATP-F68-PFC.

In some embodiments, the nanoemulsion droplets include chemically modified TAT peptides attached to the surfactant to display the hydrophilic and positively charged cell penetrating moiety on the nanoemulsion surface.

In some embodiments, the average size of a nanoemulsion particle (such as those including poloxamer surfactants) ranges from about 170 nm to about 210 nm, e.g., about 170 nm to about 210 nm, about 170 nm to about 190 nm, about 175 nm to about 210 nm, about 180 nm to about 210 nm, about 170 nm to about 200 nm, and about 170 nm to about 195 nm. Methods for determining nanoemulsion size includes using dynamic light scattering methods known to those skilled in the art.

In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm after synthesis. In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm at day 1, day 5, day 10, day 15, day 20, day 25, day 30, day 35, day 40, day 45, day 50 or more after synthesis.

In some embodiments, the average size of a nanoemulsion particle comprising 1.5%-20% (w/w) TAT ranges from about 170 nm to about 210 nm, e.g., about 170 nm to about 210 nm, about 170 nm to about 190 nm, about 175 nm to about 210 nm, about 180 nm to about 210 nm, about 170 nm to about 200 nm, and about 170 nm to about 195 nm. In certain instances, the nanoemulsion particle comprises about 1.5%-20% (w/w) TAT, e.g., about 1.5%-20% (w/w) TAT, about 1.5%-15% (w/w) TAT, about 2.0%-20% (w/w) TAT, about 2.0%-15% (w/w) TAT, about 1.5%-13% (w/w) TAT, about 2.0%-13% (w/w) TAT, about 1.5%-10% (w/w) TAT, about 2.0%-10% (w/w) TAT, about 1.5% (w/w) TAT, about 2.0% (w/w) TAT, about 2.5% (w/w) TAT, about 3.0% (w/w) TAT, about 3.5% (w/w) TAT, about 4.0% (w/w) TAT, about 4.5% (w/w) TAT, about 5.0% (w/w) TAT, about 5.5% (w/w) TAT, about 6.0% (w/w) TAT, about 6.5% (w/w) TAT, about 7.0% (w/w) TAT, about 7.5% (w/w) TAT, about 8.0% (w/w) TAT, about 8.5% (w/w) TAT, about 9.0% (w/w) TAT, about 9.5% (w/w) TAT, about 10.0% (w/w) TAT, about 10.5% (w/w) TAT, about 11.0% (w/w) TAT, about 11.5% (w/w) TAT, about 12.0% (w/w) TAT, about 12.5% (w/w) TAT, about 13.0% (w/w) TAT, about 13.5% (w/w) TAT, about 14.0% (w/w) TAT, about 14.5% (w/w) TAT, about 15.0% (w/w) TAT, about 15.5% (w/w) TAT, about 16.0% (w/w) TAT, about 16.5% (w/w) TAT, about 17.0% (w/w) TAT, about 17.5% (w/w) TAT, about 18.0% (w/w) TAT, about 18.5% (w/w) TAT, about 19.0% (w/w) TAT, about 19.5% (w/w) TAT, and about 20.0% (w/w) TAT.

In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm after synthesis. In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm at day 1, day 5, day 10, day 15, day 20, day 25, day 30, day 35, day 40, day 45, day 50 or more after synthesis.

In some embodiments, the average size of a nanoemulsion particle is about 170 nm, about 171 nm, about 172 nm, about 173 nm, about 174 nm, about 175 nm, about 176 nm, about 177 nm, about 178 nm, about 179 nm, about 180 nm, about 181 nm, about 182 nm, about 183 nm, about 184 nm, about 185 nm, about 186 nm, about 187 nm, about 188 nm, about 189 nm, about 190 nm, about 191 nm, about 192 nm, about 193 nm, about 194 nm, about 195 nm, about 196 nm, about 197 nm, about 198 nm, about 199 nm, about 200 nm, about 201 nm, about 202 nm, about 203 nm, about 204 nm, about 205 nm, about 206 nm, about 207 nm, about 208 nm, about 209 nm, or about 210 nM.

In some embodiments, the PDI value ranges from about 0.050 to about 0.14, as measured using standard light scattering methods. In some embodiments, the PDI value ranges from about 0.050 to about 0.14, e.g., about 0.05-0.14, about 0.05-0.12, about 0.05-0.10, about 0.06-0.14, about 0.06-0.13, about 0.06-0.12, about 0.06-0.11, about 0.06-0.10, about 0.07-0.14, about 0.07-0.13, about 0.07-0.12, about 0.07-0.11, about 0.07-0.10, about 0.0795-0.095, 0.0795-0.10, 0.0795-0.11, 0.0795-0.12, 0.0795-0.12, 0.0795-0.13, and 0.0795-0.14. In some embodiments, the PDI value ranges from about 0.050 to about 0.14 at day 1, day 5, day 10, day 15, day 20, day 25, day 30, day 35, day 40, day 45, day 50 or more after synthesis.

In some embodiments, the average size of a nanoemulsion particle is about 180 nm with a polydispersity index (PDI) of about 0.0795-0.095, as measured using standard light scattering methods known in the art. In some embodiments, the particle size does not separate into fluorous and aqueous phases over at least 3 months or more. In some embodiments, the average particle size slightly increases after synthesis. In some cases, the average particle size increases by an average of about 9% by day 45 post-synthesis. In some instances, the average particle size stabilizes after about 10 days, 20 days, 30 days, 40 days, 50 days, 60 days or more after synthesis. In some embodiments, the nanoemulsions comprising TATA were stable for at least 2 months at 4° C. In some embodiments, the nanoemulsions comprising TATP were stable for at least 2 months at 4° C.

In one aspect, provided herein is a method for producing a cell penetrating peptide-PFC nanoemulsion comprising a poloxamer surfactant. In some embodiments, the method comprises the following procedure: (a) 3.78 g of polyethylene-polypropylene (F68, 1 equiv, 0.0453 mmol, mol wt=8350 g/mol), purchased as a solid, is dried to a powder under high vacuum for 1 hour prior to use; (b) 25 mL of anhydrous dichloromethane is added to the powder and stirred until dissolution, and thus yielding a clear solution; (c) the reaction is maintained under dry conditions using a steady stream of N₂ gas, and 172 mg of 6-maleimidohexanoic acid (1.8 equiv, 0.815 mmol) is added in one portion yielding a pale yellow solution; (d) 0.906 mL of N, N′-Dicyclohexylcarbodiimide is added dropwise to yield a cloudy solution and a precipitate; (d) the reaction is stirred overnight at room temperature under inert gas and can be monitored by thin layer chromatography (TLC) with product retention factor (R_{f})=0.1 in 20:80:0.5 mix of MeOH:CHCl₃:AcOH; (e) the reaction byproduct is removed by filtration and an excess of hexanes is added to the filtrate causing the product to precipitate, which is then collected by filtration; (f) the product can be a pale pink solid, 10 (yield=2.183 g, mol wt=8493 g/mol); (g) to 2.5 mg of 10 (1 equiv, 0.00029 mmol) which is prepared as a 5 mg/mL solution in distilled water, 0.3 mg of Cys-TAT (0.68 equiv, 0.00020 mmol) which is prepared as a 2 mg/mL solution in HEPES buffer is added and stirred overnight; (h) 10 equiv of cysteine is added to cap any remaining maleimide groups; and (i) the contents of the reaction vessel is dialyzed to remove residual Cys-Tat (mol wt=1661.99 g/mol) and cysteine (mol wt=121 g/mol).

Also provided herein is a method for preparing a CPP surfactant (e.g., TAT-poloxamer). In some embodiments, the method comprises the following procedure: (a) 1 mmol of 1H,1H-perfluoro-1-heptanol (0.350 g, 1 mmol, mol wt=350 g/mol) or 1H,1H-perfluoro-3,6,9-trioxadecan-1-ol (0.398 g, 1 mmol, mol wt=398 g/mol) is added to a 25 mL round bottom flask along with 232 mg 6-maleimidohexanoic acid (232 mg, 1.1 equiv, 1.1 mmol, mol wt=211.32 g/mol); (b) anhydrous dichloromethane (5 mL) is added and the flask is maintained under a constant stream of N₂ gas while stirring; (c) once the reactants are dissolved, 572.4 mg benzotriazol-1-yl-oxytripyrrolidinophosphonium hexafluorophosphate (PyBOP, 572.4 mg, 1.1 equiv, 1.1 mmol, mol wt=520.39 g/mol) is added in one portion and after 2 min for the coupling reagent to dissolve, 350 μL of diisopropylethylamine (DIEA, 2 equiv, 2 mmol) is added to start the reaction, and the flask is left under a slow N₂ stream with constant stirring at room temperature for 16 hours; (d) the reaction completion can be monitored by thin layer chromatography (TLC, R_{f}=0.4, 3:7 EtOAc:hexanes), and purification and solvent removal is accomplished using a Combiflash Rf Lumen silica gel column (12 g, silica Redisep column) using a hexane and ethyl acetate gradient with 1:0 hexane:EtOAc for 3 min, followed by an increase in polarity to 1:1 hexane:EtOAc from 3 min to 14 min, followed by a 0:1 hexane:EtOAc wash for 1 min. In some embodiments, the method also includes the following steps: (i) using an evaporative light scattering (ELS) detector for monitoring product peaks, at 250 nm and 280 nm wavelengths, which elute at retention times (t_{R})=9 min (1) and t_{R}=10.5 min (2), respectively; (ii) the collected fractions are concentrated with a rotary evaporator and are dried on high vacuum overnight. In some instances, the products can be a clear oil with mol wt=591.28 g/mol (1) (yield=325 mg) and mol wt=543.28 g/mol (2) (yield=380.1 mg). In some embodiments, a method for synthesizing a poloxamer-TAT conjugate is described in Example 1 below.

In some embodiments, the method for producing a cell penetrating peptide-PFC nanoemulsion also includes the following procedure: (1) Cys-TAT.9TFA (30 mg, 0.016 mmol, 1 equiv, mol wt=2688.16 g/mol) is dissolved in 464 μL of 0.05% TFA-water and a solution of 1 or 2 (0.014 mmol) in trifluoroethanol (556 μL) is then added to the solution of Cys-TAT, followed by the addition of 116 μL of 1 M 3-(N-morpholino)propanesulfonic acid (MOPS) buffer at pH=7.4; (2) in some instances, the reaction completion is assessed by liquid chromatography mass spectroscopy (LC-MS, Model 1100 with LC/MSD Trap) using a 95:5 gradient of water+0.05% TFA:acetonitrile+0.05% TFA for 5 min, then 95:5 to 10:90 in 20 min, followed by 10:90 to 0:100 in 10 min, t_{R}=18.5 min (1) and t_{R}=18.6 min (2)] and stopped after 30 min by addition of 100 μL of glacial acetic acid; (3) following filtration (using a 0.22 μm nylon filter), the crude mixture is purified by semi-prep high pressure liquid chromatography [HPLC, gradients can be used: 90:10 descending to 10:90 water+0.05% TFA, acetonitrile+0.05% TFA in 20 min, t_{R}=12.5 min, m/z=1127.4, mol wt=2254.28 g/mol (1a, TATP) and t_{R}=13.7, m/z=1103.5, mol wt=2206.28 g/mol (2a, TATA)].

In some embodiments, the method for preparing nanoemulsions includes the following procedure: (a) a 5% w/w ratio of total surfactant to PFC is used and for 4 mL of nanoemulsion product, 40 mg of polyethylene-polypropylene (Pluronic® F68) in 400 μL of water is added to a glass vial containing 465 μL PFCE; (b) to the solution, 4 mg (1.21 μmol) of TATP (1a) or TATA (2a) (1.23 μmol) is added followed by 3.135 mL of purified water; (c) the solution is ultrasonicated (30% power, 1 min, using, for instance, an Omni Ruptor 250W) and then is passed through a microfluidizer (LV1) at 10,000 psi pressure four times; and (d) the TATA- and TATP-F68-PFC (3) nanoemulsions are sterile filtered using a 0.22 μm syringe filter (such as one from Acrodisc PF) and bottled in autoclaved glass vials. The capped vials can be stored at 4° C. until use.

In some embodiments, a detectable moiety is incorporated in a poloxamer containing nanoemulsion. In some embodiments, the detectable moiety comprises a fluorescent moiety, a luminescent moiety, a phosphorescent moiety, a fluorescence-quenching moiety, a radioactive moiety, a radiopaque moiety, a paramagnetic moiety, a contrast agent, or a combination thereof. In some embodiments, the fluorescent moiety comprises a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are disclosed, for example, in U.S. Pat. Nos. 4,452,720; 5,227,487; 5,543,295; 7,329,735; 7,906,636; and 9,695,251, the disclosures are herein incorporated by reference in their entirety, including the formulas and figures.

Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. Nos. 6,008,379; 5,750,409; 5,066,580, and 4,439,356. A useful dye may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS REDO), and other rhodamine dyes. Other rhodamine dyes can be found, for example, in U.S. Pat. Nos. 6,080,852; 6,025,505; 5,936,087; 5,750,409. Another useful dye may include a cyanine dye, such as, for example, Cy3, Cy3B, Cy3.5, Cy5, Cy5.5, Cy7.

In some embodiments, a fluorescent moiety is a fluorescent label. In some embodiments, a fluorescent label is indocarbocyanine dye, Cy5, Cy5.5, Cy7, IRDYE 800CW, ALEXA647, or a combination thereof. In some embodiments, a detectable moiety is a MRI contrast agent. In some embodiments, a detectable moiety is Gd complex of [4,7,10-tris(carboxymethyl)-1,4,7,10-tetraazacyclododec-1-yl]acetyl.

Some of the above compounds or their derivatives will produce phosphorescence in addition to fluorescence, or will only phosphoresce. Some phosphorescent compounds include porphyrins, phthalocyanines, polyaromatic compounds such as pyrenes, anthracenes and acenaphthenes, and so forth, and may be, or may be included in, a cargo moiety. A cargo moiety may also be or include a fluorescence quencher, such as, for example, a (4-dimethylamino-phenylazo)benzoic acid (DABCYL) group.

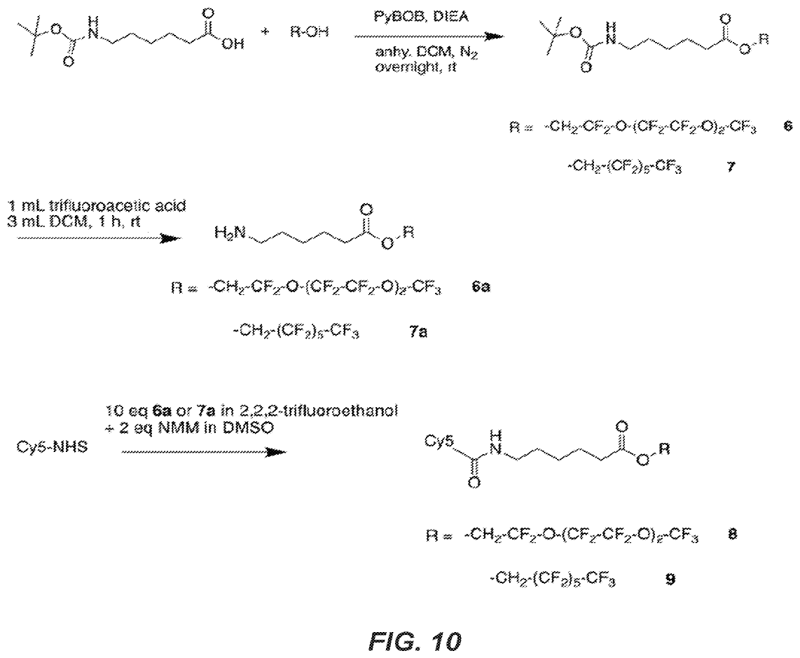

In one aspect, a labeled nanoemulsion is prepared with a detectable dye (e.g., Cy5 dye) attached. Exemplary methods for preparing labeled nanoemulsions are provided in FIG. 10. An exemplary method comprises the following steps: (a) to 0.5 mmol of 1H,1H-Perfluoro-1-heptanol (mol wt=350.08 g/mol) or 1H,1H-Perfluoro-3,6,9-trioxadecan-1-ol (mol wt=398.08 g/mol), 0.55 mmol of 6-(Boc-amino)caproic acid (mol wt=231.29 g/mol) is added and this mixture is dissolved in a minimum amount of dry dichloromethane (DCM), and the reaction mix is stirred under N2 gas; (b) 0.55 mmol pyBOB (benzotriazol-1-yl-oxytripyrrolidinophosphonium hexafluorophosphate) is added, followed by 0.74 mmol of diisopropylethylamine (DIEA), and the mixture is stirred under inert gas overnight at room temperature (in some cases, reaction completion is monitored by TLC); (c) the solvent is removed with a rotary evaporator, and the sample is dissolved in minimum amount of DCM; (d) wet crude sample is loaded on a 4 gm silica gel Redisep column for purification using a Combiflash Rf Lumen; (e) product was eluted with 100% hexane for 2 min, then 70%:30% EtOAc:hexane over 10 min, and the desired product 6 or 7 is eluted between 30-40% EtOAc and tR=6-7.5 min, as monitored by ELS detector. The method also comprises the following steps: (1) a 25 mM stock solution of 6a or 7a is prepared by dissolving weighed oil in a calculated amount of trifluoroethanol, for instance, 8 μL of 6 mM Cy5-N-hydroxysuccinimide (Cy5-NHS, 48 nmol, GE Healthcare, Chicago, Ill.) and an excess of 6a or 7a (approximately 20 equiv, 960 nmol or 38.5 μl of 25 mM stock prepared above) is added; (2) the molar equivalent amount of N-methyl morpholine is added and prepared as a 50 mM solution in DMSO, and the reaction is stirred at room temperature overnight; (3) afterwards, 2 μl of acetic acid is added, and the reaction mix is purified by HPLC (gradient 10:90 to 90:10 water+0.05% TFA:Acetonitrile+0.05% in 20 min and retain at 90:10 for an additional 10 min on a Phenomenex Luna 5 μm C18(2) 100 Å, 250×10 mm column); and (4) the desired product is eluted at t_{R}=21.6 min, m/z=1150.3 (8) and t_{R}=20.3 min, m/z=1102.3 min (9), as monitored by UV absorbance at 650 nm.

To prepare labeled TATP nanoemulsions, prior to sonication, 0.3 μM of 8 is added to a labeling cocktail. Similarly, to prepare labeled TATA nanoemulsions 0.3 μM of 9 is added to a cocktail. Following sonication and microfluidization a faint blue nanoemulsion is obtained.

### PFC Nanoemulsion Comprising CPP and Phospholipid Surfactants

Provided herein is a method of synthesizing peptide-PFC nanoemulsion comprising a phospholipid surfactant. In some embodiments, the peptide-PFC nanoemulsion comprises a hydrophilic anchor, a perfluorocarbon and a phospholipid surfactant. In some embodiments, the nanoemulsion comprises a hydrophilic anchor, a perfluorocarbon and a phospholipid surfactant.

In some embodiments, the peptide-PFC nanoemulsion comprises a cell penetrating peptide, a perfluorocarbon and a phospholipid surfactant. In some embodiments, the peptide-PFC nanoemulsion comprises a transactivating transcription peptide, a perfluorocarbon and a phospholipid surfactant. In some embodiments, the peptide-PFC nanoemulsion comprises a transactivating transcription peptide of a virus (such as but not limited to HIV), a perfluorocarbon and a phospholipid surfactant. In some instances, the perfluorocarbon includes perfluoropolyether (PFPE) or perfluoro-15-crown-5-ether (PFCE). In some embodiments, the phospholipid surfactant comprises an egg york phospholipid (EYP). In some embodiments, the nanoemulsion comprises a detectable moiety.

In some embodiments, the nanoemulsion comprises a TAT peptide, PFCE, and an egg york phospholipid. In some embodiments, the nanoemulsion comprises a TAT peptide, PFPE, and an egg york phospholipid. In some embodiments, the nanoemulsion is synthesized by direct insertion of the peptide conjugate into a phospholipid. In some embodiments, the nanoemulsion is synthesized by post-insertion of the peptide conjugate into a phospholipid. As described above, exemplary TAT peptides include an amino acid sequence as set forth in, for example, Uniprot No. P04608 and NCBI Ref. Seq. No. NP_057853.1. Useful TAT doped PFC nanoemulsions include a phospholipid-TAT-PFC, as provided in the figures including FIG. 1B—a scheme for TAT-phospholipid anchor conjugation for EYP surfactant formulated nanoemulsions. Such nanoemulsions are formed with EYP surfactant, where cys-TAT was conjugated to 1,2-Distearoyl-sn-glycero-3-phosphoethanolamine-peg2000-maleimide. TAT-modified pegylated phospholipid incorporated into EYP-surfactant nanoemulsions are useful for labeling cells.

In some embodiments, nanoemulsions comprise up to about 0.15 mol % of EYP. In some embodiments, nanoemulsions comprise about 0.01 mol % to about 0.15 mol % of EYP, e.g., about 0.01 mol % to about 0.15 mol %, about 0.01 mol % to about 0.14 mol %, about 0.01 mol % to about 0.13 mol %, about 0.01 mol % to about 0.12 mol %, about 0.01 mol % to about 0.11 mol %, about 0.01 mol % to about 0.10 mol %, about 0.01 mol % to about 0.09 mol %, about 0.05 mol % to about 0.15 mol %, about 0.05 mol % to about 0.10 mol %, about 0.10 mol % to about 0.15 mol %, and about 0.02 mol % to about 0.05 mol % of EYP.

In some embodiments, the average size of a nanoemulsion particle (such as those including phospholipid surfactants) ranges from about 170 nm to about 210 nm, e.g., about 170 nm to about 210 nm, about 170 nm to about 190 nm, about 175 nm to about 210 nm, about 180 nm to about 210 nm, about 170 nm to about 200 nm, and about 170 nm to about 195 nm.

In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm after synthesis. In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm at day 1, day 5, day 10, day 15, day 20, day 25, day 30, day 35, day 40, day 45, day 50 or more after synthesis.

In some embodiments, the average size of a nanoemulsion particle comprising 1.5%-20% (w/w) TAT ranges from about 170 nm to about 210 nm, e.g., about 170 nm to about 210 nm, about 170 nm to about 190 nm, about 175 nm to about 210 nm, about 180 nm to about 210 nm, about 170 nm to about 200 nm, and about 170 nm to about 195 nm. In certain instances, the nanoemulsion particle comprises about 1.5%-20% (w/w) TAT, e.g., about 1.5%-20% (w/w) TAT, about 1.5%-15% (w/w) TAT, about 2.0%-20% (w/w) TAT, about 2.0%-15% (w/w) TAT, about 1.5%-13% (w/w) TAT, about 2.0%-13% (w/w) TAT, about 1.5%-10% (w/w) TAT, about 2.0%-10% (w/w) TAT, about 1.5% (w/w) TAT, about 2.0% (w/w) TAT, about 2.5% (w/w) TAT, about 3.0% (w/w) TAT, about 3.5% (w/w) TAT, about 4.0% (w/w) TAT, about 4.5% (w/w) TAT, about 5.0% (w/w) TAT, about 5.5% (w/w) TAT, about 6.0% (w/w) TAT, about 6.5% (w/w) TAT, about 7.0% (w/w) TAT, about 7.5% (w/w) TAT, about 8.0% (w/w) TAT, about 8.5% (w/w) TAT, about 9.0% (w/w) TAT, about 9.5% (w/w) TAT, about 10.0% (w/w) TAT, about 10.5% (w/w) TAT, about 11.0% (w/w) TAT, about 11.5% (w/w) TAT, about 12.0% (w/w) TAT, about 12.5% (w/w) TAT, about 13.0% (w/w) TAT, about 13.5% (w/w) TAT, about 14.0% (w/w) TAT, about 14.5% (w/w) TAT, about 15.0% (w/w) TAT, about 15.5% (w/w) TAT, about 16.0% (w/w) TAT, about 16.5% (w/w) TAT, about 17.0% (w/w) TAT, about 17.5% (w/w) TAT, about 18.0% (w/w) TAT, about 18.5% (w/w) TAT, about 19.0% (w/w) TAT, about 19.5% (w/w) TAT, and about 20.0% (w/w) TAT.

In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm after synthesis. In some embodiments, the average size of a nanoemulsion particle ranges from about 170 nm to about 210 nm, e.g., about 170 nm to about 210 nM, about 170 nm to about 190 nm, about 175 nm to about 210 nm, about 180 nm to about 210 nm, about 170 nm to about 200 nm, and about 170 nm to about 195 nm at day 1, day 5, day 10, day 15, day 20, day 25, day 30, day 35, day 40, day 45, day 50 or more after synthesis.

In some embodiments, the average size of a nanoemulsion particle is about 170 nm, about 171 nm, about 172 nm, about 173 nm, about 174 nm, about 175 nm, about 176 nm, about 177 nm, about 178 nm, about 179 nm, about 180 nm, about 181 nm, about 182 nm, about 183 nm, about 184 nm, about 185 nm, about 186 nm, about 187 nm, about 188 nm, about 189 nm, about 190 nm, about 191 nm, about 192 nm, about 193 nm, about 194 nm, about 195 nm, about 196 nm, about 197 nm, about 198 nm, about 199 nm, about 200 nm, about 201 nm, about 202 nm, about 203 nm, about 204 nm, about 205 nm, about 206 nm, about 207 nm, about 208 nm, about 209 nm, or about 210 nM.

In some embodiments, the PDI value ranges from about 0.050 to about 0.14, as measured using standard light scattering methods. In some embodiments, the PDI value ranges from about 0.050 to about 0.14, e.g., about 0.05-0.14, about 0.05-0.12, about 0.05-0.10, about 0.06-0.14, about 0.06-0.13, about 0.06-0.12, about 0.06-0.11, about 0.06-0.10, about 0.07-0.14, about 0.07-0.13, about 0.07-0.12, about 0.07-0.11, about 0.07-0.10, about 0.0795-0.095, 0.0795-0.10, 0.0795-0.11, 0.0795-0.12, 0.0795-0.12, 0.0795-0.13, and 0.0795-0.14. In some embodiments, the PDI value ranges from about 0.050 to about 0.14 at day 1, day 5, day 10, day 15, day 20, day 25, day 30, day 35, day 40, day 45, day 50 or more after synthesis.

In some embodiments, the average size of a nanoemulsion particle is about 180 nm with a polydispersity index (PDI) of about 0.0795-0.095, as measured using standard light scattering methods known in the art. In some embodiments, the particle size does not separate into fluorous and aqueous phases over at least 3 months or more. In some embodiments, the average particle size slightly increases after synthesis. In some cases, the average particle size increases by an average of about 9% by day 45 post-synthesis. In some instances, the average particle size stabilizes after about 10 days, 20 days, 30 days, 40 days, 50 days, 60 days or more after synthesis. In some embodiments, the nanoemulsions comprising phospholipid surfactants were stable for at least 2 months at 4° C.

In one aspect, provided herein is a method for producing a cell penetrating peptide-PFC nanoemulsion comprising a phospholipid surfactant.

In some embodiments, the method comprises preparing a CPP-phospholipid surfactant. In some embodiments, the following method includes the following procedure: (a) 1,2-distearoyl-sn-glycero-3-phosphoethanolamine-N-[maleimide(polyethyleneglycol)-2000] (DSPE-PEG2000-maleimide, 1.8 equiv., 0.00217 mmol, 6.5 mg) is suspended in HEPES buffer (0.05 M, 500 μL, pH=7.5) by sonication; (b) a fresh solution of Cys-TAT (1 equiv, 0.0012 mmol, 2.0 mg) in HEPES buffer (0.05 M, 300 μL, pH=7.5) is added in one portion, and the mixture is agitated on a shaker at 37° C. for 6 hours; (c) 2-Mercaptoethanol (0.8 μL, 10 equiv, 0.012 mmol) is added to react with any remaining maleimide groups, and the solution is agitated further for 30 min; (d) the conjugate is de-salted and purified in deionized water using a dialysis cassette (such as a Slide-A-Lyzer #2K MWCO, cassette size=3 mL) at room temperature; (e) water is replaced at about 2, 4 and 22 hours (volume 300:1 compared to cassette size); (f) the sample is recovered from the dialysis cassette and in some instances, is analyzed by matrix assisted laser desorption/ionization (MALDI) mass spectrometry, such that as a mixture of the desired product is identified—DSPE-PEG(2000)-Cys-TAT (mol wt=4660 g/mol) 4 and DSPE-PEG(2000)-mercaptoethanol (mol wt=3020 g/mol); and (g) the solution is lyophilized to a dry powder to give a near-quantitative yield of the desired product, 4.

Provided herein are methods for preparing nanoemulsions includes incorporating phospholipid-PEG-TAT conjugates into egg yolk phospholipids (EYPs). In some embodiments, the method for direct insertion of the peptide conjugate into the egg yolk phospholipid comprises the following steps: (1) compound 1 (2.8 mg, 0.6 μmol) and EYP (304 mg, 0.4 mmol, Sigma Aldrich) are mixed resulting in a TAT to lipid surfactant ratio of 0.15 mol %; (2) PFPE oil (1.18 g, 0.87 mmol, mol wt=1300-1400 g/mol, Exfluor) is added to obtain a 26% w/w ratio of phospholipid to PFPE; (3) sterile water is added to obtain a 120-150 mg/mL concentration of PFPE; and (4) the nanoemulsions are sterile-filtered through a 0.2 μm filter into glass vials, capped, and stored at 4° C. until use. Each nanoemulsion can be characterized by dynamic light scattering (DLS) particle analysis and ¹⁹F NMR.

The method also includes the following: (a) compound 4 is added to a solution of EYP in chloroform (5 mL), vortexed on medium for 1 min, and the resulting solution evaporated with a stream of nitrogen while manually rotating the vessel; (b) the vial is then placed under high vacuum overnight to give a dry lipid film; (c) sterile water is added to hydrate the lipid film for 5 min followed by vortexing on medium for 2 min and then ultrasonication (30% power, 4 min); (d) PFPE is added to the vial in one portion, vortexed briefly, and then ultrasonicated (30% power, 2 min); and (e) the crude emulsion (5) is passed four times through a microfluidizer at 20,000 psi with the reaction chamber cooled on ice.

In some embodiments, the method for post-insertion of the peptide conjugate into the egg yolk phospholipid comprises the following steps: (1) compound 1 (2.8 mg, 0.6 μmol) and EYP (304 mg, 0.4 mmol, Sigma Aldrich) are mixed resulting in a TAT to lipid surfactant ratio of 0.15 mol %; (2) PFPE oil (1.18 g, 0.87 mmol, mol wt=1300-1400 g/mol, Exfluor) is added to obtain a 26% w/w ratio of phospholipid to PFPE; (3) sterile water is added to obtain a 120-150 mg/mL concentration of PFPE; and (4) the nanoemulsions are sterile-filtered through a 0.2 μm filter into glass vials, capped, and stored at 4° C. until use. Each nanoemulsion can be characterized by dynamic light scattering (DLS) particle analysis and ¹⁹F NMR.

The method also includes the following: (a) forming a suspension of EYP in sterile water by ultrasonication (30% power, 4 min); (b) PFPE oil is added to the vial in one portion, vortexed briefly, and then ultrasonicated (30% power, 2 min); (c) the crude emulsion is passed four times through a microfluidizer at 20,000 psi with the reaction chamber cooled on ice; (d) to incorporate TAT, solutions of 1 based on mol % of total EYP surfactant are prepared in sterile water and the solution of 1 is added to the preformed nanoemulsion and agitated on a bioshaker at 37° C. for 5 h to obtain (5) nanoemulsion.

### Emulsions

The imaging reagent used in the subject methods is a fluorocarbon, i.e., a molecule including at least one carbon-fluorine bond. By virtue of the ¹⁹F atoms, the imaging reagents disclosed herein may be detected by ¹⁹F MRI and other nuclear magnetic resonance techniques, such as MRS techniques. In certain embodiments, a fluorocarbon imaging reagent will have one or more of the following properties: (1) reduced cytotoxicity; (2) a ¹⁹F NMR spectrum that is simple, ideally having a single, narrow resonance to minimize chemical shift artifacts; (3) high sensitivity with a large number of NMR-equivalent fluorine atoms in each molecule; and (4) formulated to permit efficient labeling of many cell types and not restricted to phagocytic cells. In some embodiments, the imaging reagent comprises a plurality of fluorines bound to carbon, e.g., greater than 5, greater than 10, greater than 15 or greater than 20 fluorines bound to carbon. In some embodiments, at least 4, at least 8, at least 12 or at least 16 of the fluorines have a roughly equivalent NMR chemical shift.

For labeling cells in culture, the imaging reagents can be employed in one or more of at least three modalities: (1) imaging reagents that are internalized or otherwise absorbed by target cells without the formation of any covalent or other binding association (first type); (2) imaging reagents that covalently attach to target cells (second type); and (3) imaging reagents coupled to molecules, such as antibodies or ligands, that bind to molecules present on the target cells (third type). In some embodiments, the imaging reagents that are internalized or otherwise absorbed by target cells without the formation of any covalent or other binding association (first type). In some embodiments, the imaging reagents that covalently attach to target cells (second type). In some embodiments, the imaging reagents coupled to molecules, such as antibodies or ligands, that bind to molecules present on the target cells (third type). In some embodiments, the imaging agent is a mixture of one or more of first, second, third types.

Imaging reagents of the first type include the perfluoro crown ethers and other perfluoropolyethers (PFPEs) that are taken up by cells and, preferably, are retained in the cell without degradation for a substantial period of time, e.g., having a half-life in the cell of at least 1 hour, at least 4 hours, at least about a day, at least about three days, or even at least about a week. In some embodiments, the imaging reagent does not interfere with ordinary cellular functions or exhibit cytotoxicity at the concentrations employed for labeling. As demonstrated herein, perfluoropolyethers show reduced toxic effect on the labeled cells.

Imaging reagents of the second type include electrophilic compounds that react with nucleophilic sites on the cell surface, such as exposed thiol, amino, and/or hydroxyl groups. Accordingly, imaging reagents such as maleimides, alkyl iodides, N-hydroxysuccinimide or N-hydroxysulfosuccinimide esters (NHS or sulfo-NHS esters), acyl succinimides, and the like can form covalent bonds with cell surfaces. Other techniques used in protein coupling can be adapted for coupling imaging reagents to cell surface proteins. See, for example, Means et al. (1990) Bioconjugate Chemistry 1:2-12, for additional approaches to such coupling.

Imaging reagents of the third type can be prepared by reacting imaging reagents of the second type not with the cells themselves, but with a functional moiety that is cell-targeting ligand or antibody. Suitable ligands and antibodies can be selected for the application of interest. For example, a ligand that selectively targets hematopoietic cells could be labeled with an imaging reagent as described herein and administered to a patient such as by infection. In some embodiments, the ligand can be a ligand that targets an immune cell.

Alternatively, an imaging reagent can be coupled to an indiscriminate internalizing peptide, such as antennapedia protein, HIV transactivating (TAT) protein, mastoparan, melittin, bombolittin, delta hemolysin, pardaxin, Pseudomonas exotoxin A, clathrin, Diphtheria toxin, C9 complement protein, or a fragment of any of these. Cells treated with this indiscriminate molecule ex vivo will absorb the imaging reagent. When such labeled cells are implanted into an animal, such as a mammal, the imaging reagent can be used to visualize and/or track the implanted cells by nuclear magnetic resonance techniques.

In one embodiment, the internalizing peptide is derived from the Drosophila antepennepedia protein, or homologs thereof. The 60-amino acid-long homeodomain of the homeo-protein antennapedia has been demonstrated to translocate through biological membranes and can facilitate the translocation of heterologous polypeptides to which it is coupled. See, for example, Derossi et al, (1994) J Biol Chem 269:10444-10450; and Perez et al. (1992) J Cell Sci 102:717-722. It has been demonstrated that fragments as small as 16 amino acids long of this protein are sufficient to drive internalization. See, for example, Derossi et al, (1990) J Biol Chem 271:18188-18193.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol 63:1-8). Peptides or analogs that include a sequence present in the highly basic region can be conjugated to fluorinated imaging reagents to aid in internalization and targeting those reagents to the intracellular milieu.

The present invention provides novel compositions comprising imaging reagents. For example, the present invention provides an aqueous composition comprising perfluoro-15-crown-5 ether or PFPE oxide, an emulsifier, a surfactant co-mixture, and an additive, in certain embodiments, the surfactant co-mixture comprises lecithin (i.e., lipoid egg phosphatidyl choline), cholesterol, and dipalmltoyl phosphatidylethanolamine (DPPE). In certain such embodiments, the surfactant co-mixture comprises 70 mol % of lecithin; 28 mol % of cholesterol; and 2 mol % of DPPE. In certain embodiments, the additive is propylene glycol.

As used herein, the term “PFPE oxide” refers to perfluoropoly(ethylene glycol) Dialkyl Ether (e.g., commercially available and can be purchased from Exfluor Inc., TX).

In certain embodiments, the emulsifier is also a non-ionic solubilizer. In certain embodiments, the emulsifier comprises glycerol polyethylene glycol ricinoleate. In certain such embodiments, the emulsifier further comprises fatty acid esters of polyethylene glycol, free polyethylene glycols, and ethoxylated glycerol. In certain embodiments, the emulsifier is prepared by reacting castor oil and ethylene oxide in a molar ratio of 1:35. Exemplary emulsifiers can be obtained from BASF Corporation and are sold under the trade name of Cremophor EL®.

In certain embodiments, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises perfluor-15-crown-5 ether or PFPE oxide in the range of 20% to 50% w/v, such as 25% to 45% w/v, such as 30% to 40% w/v, such as 30%, 31%, 32%, 33%, 34%, 35%, 36%, 37%, 38%, 39% or 40% w/v. In certain such embodiments, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises perfluor-15-crown-5 ether or PFPE oxide in the range of 35% to 36% w/v, such as 35.1%, 35.2%, 35.3%, 35.4%, 35.5%, 35.6%, 35.7%, 35.8%, or 35.9% w/v. In certain embodiments, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises perfluor-15-crown-5 ether pr PFPE oxide in 35.6% w/v.

In certain embodiments, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises Cremophor EL® in the range of 1% to 10% w/v, such as 1% to 5% w/v, such as 1%, 2%, 3%, 4%, or 5% w/v. In certain embodiments, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises Cremophor EL® in 3% w/v.

In certain embodiments, the aqueous composition comprising perfluoro-15-crown-5 ether or PFPE oxide, Cremophor EL®, a surfactant co-mixture (e.g., comprising lecithin, cholesterol, and DPPE), and propylene glycol comprises propylene glycol in the range of 1% to 10% w/v, such, as 1% to 5% w/v, such as 1%, 2%, 3%, 4%, or 5% w/v. In certain embodiments, the aqueous composition comprising perfluoro-15-crown-5 ether or PFPE oxide), Cremophor EL®, a surfactant co-mixture (e.g., comprising lecithin, cholesterol, and DPPE), and propylene glycol comprises propylene glycol in 2% w/v.

In certain embodiments, the aqueous composition comprising perfluoro-15-crown-5 ether or PFPE oxide, Cremophor EL®, an additive (e.g., propylene glycol), and a surfactant co-mixture, wherein the surfactant co-mixture comprises lecithin, cholesterol, and DPPE, comprises the surfactant co-mixture, wherein the surfactant co-mixture comprises lecithin, cholesterol, and DPPE, in the range of 1% to 10% w/v, such as 1% to 5% w/v, such as 1%, 2%, 3%, 4%, or 5% w/v. In certain embodiments, the aqueous composition comprising perfluoro-15-crown-5 ether or PFPE oxide, Cremophor EL®, an additive (e.g., propylene glycol), and a surfactant co-mixture, wherein the surfactant co-mixture comprises lecithin, cholesterol, and DPPE, comprises the surfactant co-mixture, wherein the surfactant co-mixture comprises lecithin, cholesterol, and DPPE, in 2% w/v.

In certain embodiments, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) further comprises polyethylamine. In certain such embodiments, the aqueous composition comprises polyethylamine in the range of 0.01% to 5.0% w/w. In certain embodiments, the aqueous composition comprising perfluoro-15-crown-5 ether or PFPE oxide, Cremophor EL®, a surfactant co-mixture (e.g., comprising lecithin, cholesterol, and DPPE), an additive (e.g., propylene glycol), and polyethylamine further comprises protamine sulfate. In certain such embodiments, the aqueous composition protamine-sulfate in the range of 0.01% to 5.0% w/w.

In certain embodiments, the present invention provides an aqueous composition comprising perfluoro-15-crown-5 ether or PFPE oxide in 35.6% w/v, Cremophor EL® in 3.0% w/v, a surfactant co-mixture (e.g., comprising lecithin, cholesterol, and DPPE) in 2.0% w/v, and an additive (e.g., propylene glycol) in 2.0% w/v.

The terms emulsion and nanoemulsion as used in this application are equivalent unless specifically stated otherwise. In certain embodiments, the emulsion may further comprise a block copolymer of polyethylene and polypropylene glycol. In certain embodiments, the emulsion may further comprise a Plutonic™ Nonionic Plutonic™ surfactants, polyethyleneoxide (PEO)/polypropyleneoxide (PPO)/polyethyleneoxide (PEO) block (ABA type), (PEO/PPO/PEO) block copolymers, exhibit a wide range of hydrophilicity/hydrophobicity as a function of the PEO/PPO ratio, so that one can expect to obtain different phase separated morphologies with polymers such as PLA as well as different degrees of hydration of the matrix. In particular, hydration plays an important role in determining polymer degradation via hydrolysis of the ester backbone. These polymeric surfactants exhibited minimal toxicities in vivo and some of them are in clinical use, as described by BASF Corporation in their 1989 Technical Bulletin; Attwood, et al., Int. J. Pharm. 26, 25 (1985); and U.S. Pat. No. 4,188,373 to Krezanoski. These materials can be obtained from BASF Corporation. In certain embodiments, emulsions of the present invention further comprise tri-block copolymer which comprises polyethyleneoxide and polypropyleneoxide.

In certain embodiments, emulsions of the present invention comprise a tri-block copolymer of poly(ethylene oxide)-poly(propylene oxide)-poly(ethylene oxide) (PEO-PPO-PEO) comprising 80% PEO content. In certain such embodiments, the hydrophilic-lipophilic balance (HLB) value of the tri-block copolymer is 29, wherein the HLB value can be calculated from the following equation:

$$HLB = - 36 \frac{m}{2 n + m} + 33$$

where n represents the number of repeat units in the PEO segment of the polymer and m represents the number of repeat units in the PPO segment of the polymer. Exemplary tri-block copolymers can be obtained, from BASF Corporation and are sold under the trade name of Pluronic™ F68.

The present invention further provides an aqueous composition comprising perfluoro-15-crown-5 ether or PFPE oxide and the Pluronic™ F68. In certain embodiments, the aqueous composition comprising perfluoro-15-crown-5 ether or PFPE-oxide and the Pluronic™ F68, comprises perfluoro-15-crown-5 or PFPE oxide ether in the range of 10% to 20% w/w, such as 12% to 1/% w/w, such as 12%, 13%, 14%, 15%, 16%, or 17% w/w. In certain embodiments, the aqueous composition comprising perfluoro-15-crown-5 ether or PFPE oxide and the Pluronic™ F68, comprises-perfluoro-15-crown-5 ether or PFPE oxide in 15% w/w. In certain embodiments, the aqueous composition comprising perfluoro-15-crown-5 ether or PFPE oxide and the Pluronic™ F68, comprises the Pluronic™ F68 in the range of 0.1% to 2.0% w/w, such as 0.1% to 1.0% w/w, such as 0.1%, 0.2%, 0.3%, 0.4%, 0.5%, 0.6%, 0.7%, 0.8%0, 0.9% or 1.0% w/w. In certain embodiments, the aqueous composition comprising perfluoro-15-crown-5 ether or PFPE oxide and the Pluronic™ F68, comprises the Pluronic™ F68 in 0.6% w/w.

In certain embodiments, the present invention provides an aqueous composition comprising perfluoro-15-crown-5 ether or PFPE oxide in 15% w/w and the Pluronic™ F68 in 0.6% w/w.

In certain embodiments, the aqueous composition comprising perfluoro-15-crown-5 ether or PFPE oxide and the Pluronic™ F68 further comprises protamine sulfate. In certain such embodiments, the aqueous composition comprising perfluoro-15-crown-5 ether or PFPE oxide, the Pluronic™ F68, and protamine sulfate comprises protamine sulfate in the range of 0.01% to 1.0% w/w, such as 0.01% to 0.5% w/w, such as 0.01% to 0.10% w/w, such as 0.01%, 0.02%, 0.03%, 0.04%, 0.05%, 0.06%, 0.07%, 0.08%, 0.09%, or 0.10% w/w. In certain embodiments, the aqueous composition comprising perfluoro-15-crown-5 ether or PFPE oxide, the Pluronic™ F68, and protamine sulfate comprises protamine sulfate in 0.04% w/w.

In certain embodiments, the aqueous composition comprising perfluoro-15-crown-5 ether or PFPE oxide and the Pluronic™ F68 further comprises polyethylamine. In certain embodiments, the present invention provides an aqueous composition comprising perfluoro-15-crown-5 ether or PFPE oxide in 15% w/w, the Pluronic™ F68 in 0.6% w/w, and protamine sulfate in 0.04% w/w.

The present invention also provides formulations of the compositions of the present invention as described above that are suitable for uptake by cells. For example, the compositions of the present invention may be formulated as an emulsion. As an example, the present invention provides an emulsion comprising an aqueous composition comprising perfluoro-15-crown-5 ether or PFPE oxide. Cremophor EL®, a surfactant co-mixture, and an additive. In certain embodiments, the surfactant co-mixture comprises lecithin, cholesterol, and dipalmitoyl phosphatidyl ethanolamine (DPPE). In certain such embodiments, the surfactant co-mixture comprises 70 mol % of lecithin; 28 mol % of cholesterol; and 2 mol % of DPPI. In certain embodiments, the additive is propylene glycol.

In certain embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises perfluor-15-crown-5 ether or PFPE oxide in the range of 20% to 50% w/v, such as 25% to 45% w/v, such as 30% to 40% w/v, such as 30%, 31%, 32%, 33%, 34%, 35%, 36%, 37%, 38%, 39% or 40% w/v. In certain such embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises perfluor-15-crown-5 ether or PFPE oxide in the range of 35% to 36% w/v, such as 35.1%, 35.2%, 35.3%, 35.4%, 35.5%, 35.6%, 35.7%, 35.8%, or 35.9% w/v. In certain embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises perfluor-15-crown-5 ether or PFPE oxide in 35.6% w/v.

In certain embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises Cremophor EL®, in the range of 1% to 10% w/v, such as 1% to 5% w/v, such as 1%, 2%, 3%, 4%, or 5% w/v. In certain embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and an additive (e.g., propylene glycol) comprises Cremophor EL® in 3% w/v.

In certain embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and propylene glycol comprises propylene glycol in the range of 1% to 10% w/v, such as 1% to 5% w/v, such as 1%, 2%, 3%, 4%, or 5% w/v. In certain embodiments of the foregoing emulsion, the aqueous composition comprising perfluoro-15-crown-5 ether or PFPE oxide, Cremophor EL®, a surfactant co-mixture (e.g., comprising lecithin, cholesterol, and DPPE), and propylene glycol comprises propylene glycol in 2% w/v.

In certain embodiments of the foregoing emulsion, the aqueous composition comprising perfluoro-15-crown-5 ether or PFPE oxide, Cremophor EL®, an additive (e.g., propylene glycol), and a surfactant co-mixture, wherein the surfactant co-mixture comprises lecithin, cholesterol, and DPPE, comprises the surfactant co-mixture, wherein the surfactant co-mixture comprises lecithin, cholesterol, and DPPE, in the range of 1% to 10% w/v, such as, 1% to 5% w/v, such as 1%, 2%, 3%, 4%, or 5% w/v. In certain embodiments of the foregoing emulsion, the aqueous composition comprising perfluoro-15-crown-5 ether or PFPE oxide, Cremophor EL®, an additive (e.g., propylene glycol), and a surfactant co-mixture, wherein the surfactant co-mixture comprises lecithin, cholesterol, and DPPE, comprises the surfactant co-mixture, wherein the surfactant co-mixture comprises lecithin, cholesterol, and DPPE, in 2% w/v.

In certain embodiments, the present invention provides an emulsion comprising an aqueous composition comprising perfluoro-15-crown-5 ether or PFPE oxide in 35.6% w/v, Cremophor EL® in 3.0% w/v, a surfactant co-mixture (e.g., comprising lecithin, cholesterol, and DPPE) in 2.0% w/v, and an additive (e.g., propylene glycol) in 2.0% w/v.

The present invention further provides an emulsion comprising an aqueous composition comprising perfluoro-15-crown-5 ether or PFPE oxide and the Pluronic™ F68. In certain embodiments of the foregoing emulsion, the aqueous composition comprising perfluoro-15-crown-5 ether or PFPE oxide and the Pluronic™ F68, comprises perfluoro-15-crown-5 ether or PFPE oxide in the range of 10% to 20% w/w, such as 12% to 17% w/w, such as 12%, 13%, 14%, 15%, 16%, or 17% w/w. In certain embodiments of the foregoing emulsion, the aqueous composition comprising perfluoro-15-crown-5 ether or PFPE oxide and the Pluronic™ F68, comprises perfluoro-15-crown-5 ether or PFPE oxide in 15% w/w. In certain embodiments of the foregoing emulsion, the aqueous composition comprising perfluoro-15-crown-5 ether or PFPE oxide and the Pluronic™ F68, comprises the Pluronic™ F68 in the range of 0.1% to 2.0% w/w, such as 0.1% to 1.0% w/w, such as 0.1%, 0.2%, 0.3%, 0.4%, 0.5%, 0.6%, 0.7%, 0.8%, 0.9% or 1.0% w/w. In certain embodiments of the foregoing emulsion, the aqueous composition comprising perfluoro-15-crown-5 ether or PFPE oxide and the Pluronic™ F68, comprises the Pluronic™ F68 in 0.6% w/w.

In certain embodiments, the present invention provides an emulsion comprising an aqueous composition comprising perfluoro-15-crown-5 ether or PFPE oxide in 15% w/w and the Pluronic™ F68 in 0.6% w/w.

In certain embodiments of the foregoing emulsion, the aqueous composition comprising perfluoro-15-crown-5 ether or PFPE oxide and the Pluronic™ F68 further comprises protamine sulfate. In certain such embodiments of the foregoing emulsion, the aqueous composition comprising perfluoro-15-crown-5 ether or PFPE oxide, the Pluronic™ F68, and protamine sulfate comprises protamine sulfate in the range of 0.01% to 1.0% w/w, such as 0.01% to 0.5% w/w, such as 0.01% to 0.10% w/w, such as 0.01%, 0.02%, 0.03%, 0.04%, 0.05%, 0.06%, 0.07%, 0.08%, 0.09%, or 0.10% w/w. In certain embodiments of the foregoing emulsion, the aqueous composition comprising perfluoro-15-crown-5 ether or PFPE oxide, the Pluronic™ F68, and protamine sulfate comprises protamine sulfate in 0.04% w/w.

In certain embodiments, the present invention provides an emulsion comprising an aqueous composition comprising perfluoro-15-crown-5 ether or PFPE oxide, in 15% w/w, the Pluronic™ F68 in 0.6% w/w, and protamine sulfate in 0.04% w/w.

In certain embodiments, the compositions and emulsions of the present invention comprise Cremophor® EL, a nonionic solubiliser and emulsifier comprising polyethylene glycol ricinoleate, made by reacting castor oil with ethylene oxide in a molar ratio of 1:35. This material can be obtained from BASF Corporation.

In certain embodiments, the emulsion may further comprise a lipid. In certain embodiments of emulsions of the present invention that further comprise a lipid, the lipid is DMPC. In certain embodiments of emulsions of the present invention that further comprise a lipid, the emulsion further comprises a Pluronic™. In certain embodiments, the Pluronic™ is F68.

In certain embodiments, the emulsion may further comprise polyethylamine. In certain embodiments, the emulsion may further comprise protamine sulfate. In certain embodiments of emulsions of the present invention that further comprise protamine sulfate, the emulsion further comprises a Pluronic™, In certain embodiments, the Pluronic™ is F68. In certain embodiments, the emulsion of the present invention further comprises protamine sulfate.

Emulsions of the present invention will preferably have a distribution of droplet sizes that allow adequate cellular uptake. In certain embodiments, a uniform droplet size may be advantageous. The desired degree of uniformity of droplet size may vary depending upon the application. In certain embodiments, the emulsion has a mean droplet size less than 500 nm, or less than 400 nm, or less than 300 nm, or less than 200 nm in diameter. Optionally, 25%, or 50%, or 75% or more of the droplets will fall within the selected range. Droplet sizes may be evaluated by, for example, light scattering techniques or by visualizing the emulsion droplets using electron microscopy micrographs. In certain cell types that have a relatively small amount of cytoplasm, such as most stem cells, the emulsions have a mean droplet size of less than 200 nm, or less than 100 nm, or less than 50 nm in diameter. In some embodiments, the nanoemulsion droplets are about 50-300 nm in mean diameter, e.g., about 50-300 nm, 50-250 nm, 50-150 nm, 50-100 nm, 100-300 nm, 100-200 nm, 100-150 nm, 110-200 nm, 120-200 nm, 130-200 nm, 140-200 nm, 150-200 nm, 150-300 nm, 160-300 nm, 170-300 nm, or about 200-300 nm in mean diameter.

In certain embodiments, small droplet size is advantageous. In certain embodiments, small droplet size increases: circulation time in applications where the emulsion is injected intravenously (iv). In certain embodiments, droplets are separable from cells by circulation. In certain embodiments, small droplet size increases ex vivo cell labeling. In certain embodiments, small droplet size increases uniform labeling.

Emulsions for use in cells should preferably be stable at a wide range of temperatures. In certain embodiments, emulsions will be stable at body temperature (37° C. for humans) and at a storage temperature, such as 4° C. or room temperature (20-25° C.). For example, it will often be desirable to store the emulsion at a cool temperature, in the range of 2-10° C., such as 4° C., and then warm the emulsion to room temperature (e.g., 18 to 28° C., and more typically 20 to 25° C.). After labeling of cells, the emulsion will experience a temperature of about 37° C. Accordingly, a emulsion will retain the desired range of droplet sizes at temperatures ranging from refrigeration temperatures up to body temperature. In certain embodiments, the emulsion is stable at temperatures ranging from 4° C. to 37° C.

The properties of an emulsion may be controlled primarily by the properties of the imaging reagent itself, the nature of surfactants and/or solvents used, and the type of processing device (e.g., sonicator, microfluidixer, homogenixer, etc.). Methods for forming emulsions with certain PFPE molecules are extensively described in U.S. Pat. Nos. 5,330,681 and 4,990,283; herein incorporated by reference in their entireties. A continuous phase of a polyhydroxylated compound, such as polyalcohols and saccharides in concentrated aqueous solution may be effective. The following polyalcohols and saccharides have proved to be particularly effective; glycerol, xylitol, mannitol, sorbitol, glucose, fructose, saccharose, maltitol, dimer compounds of glycerol (di-glycerol or bis(2,3-dihydroxypropyl) ether, solid water soluble polyhydroxylated compounds as sugars and glycerol condensation products as triglycerol and tetraglycerol. The dispersion in emulsion may be performed in the presence of conventional surfactants, including cationic, anionic, amphoteric and non-ionic surfactants. Examples of suitable surfactants include sodium lauryl sulphate, sulphosuccinate (sulphosuccinic hemiester), coco-amphocarboxyglycinate, potassium cetyl phosphate, sodium alkyl-polyoxyethylene-ether carboxylate, potassium benzalconium chloride, alkyl amidopropyl betaine, cetyl-stearilic ethoxylated alcohol, and sorbitan-ethoxylate(20)-mono-oleate Tween 20. While thermodynamic equations may be used to attempt to predict mixtures of imaging reagents that will give emulsions having the desired droplet sizes and stability, it is generally accepted that actual testing of various mixtures will be most effective. The emulsification of mixtures is simple and quick, permitting rapid testing of a wide range of combinations to identify those that give rise to emulsions that are suitable for use in the methods disclosed herein.

In the applications involving ex vivo labeling, some emulsions are designed to facilitate uptake of the imaging reagent by the subject cells. A surfactant may be designed to form stable emulsions that carry a large quantity of perfluoro-15-crown-5 ether or PFPE oxide into the aqueous phase. Additionally, it may have properties that increase the intracellular delivery of the emulsion droplets in the shortest possible incubation time. Increasing the perfluoro-15-crown-5 ether or PFPE oxide intracellular loading improves sensitivity to the labeled cells. Furthermore, minimizing the culture time can be important when working with the primary cells cultures. The efficiency of intracellular uptake depends on cell type. For example macrophages and some dendritic cells will endocytose almost any particulate, whereas other cell types of interest may only be weakly phagocytic. In either case the uptake efficiency can be boosted substantially by designing the surfactant so that the surface of the emulsion droplet has properties that promote cellular uptake in culture (i.e., “self-delivering” emulsion droplets) (see Janjie et al, JACS, 2008, 130 (9), 2832-2841 and U.S. Provisional Patent Application 61/062,710, both of which are incorporated by reference in their entirety). The emulsion droplet surface can be made to have lipophilic, or optionally cationic, properties via appropriate surfactant design. For example the surfactant can incorporate lipids, such as cationic or neutral lipids, oil-in-water colloidal emulsions, micelles, mixed micelles, or liposomes, that tend to bind to or fuse with the cell's surface, thereby enhancing emulsion droplet uptake. The emulsion droplet surface may also incorporate cell delivery signals such as polyamines. Examples include emulsions that have polyamines, such as polyethylenimine or protamine sulfate, incorporated into the emulsion droplet's surfactant layer during processing.

In certain embodiments, a colloidal system for use as a delivery vehicle in vitro and in vivo is a liposome (i.e., an artificial membrane vesicle). The preparation and use of such systems is well known in the art. Suitable cationic lipids are described in the following and are herein incorporated in their entirety; Felgner et al., 1987, PNAS 84, 7413-7417; U.S. Pat. Nos. 4,897,355; 5,279,833; 5,283,185; 5,334,761; 5,527,928; Bailey et al., U.S. Pat. Nos. 5,552,155; and 5,578,475). Other approaches include incorporation into the surfactant peptides (e.g. oligo-Arg9 and TAT-like peptides) that facilitate entry into cells, or antibodies that target specific cell surface molecules. Additionally, in certain embodiments, one can incorporate small cationic proteins into the surfactant, such as protamine sulfate, to enhance cellular uptake. Protamine sulfate is non-toxic to cells and has FDA approval for use in humans as a heparin antagonist. In certain embodiments, colloidal dispersion systems are used, such as macromolecule complexes, nanocapsules, microspheres, and beads. Other approaches for enhancing uptake of the emulsified fluorocarbons, such as by using additional transfection agents or by using electroporation of the cells, is described herein.

In some embodiments, emulsions have “self-delivering” properties without having to add uptake enhancing reagents. The emulsions are preferably stable and have a shelf-life of a period of months or years. In some embodiments, the stability is 3 months, 6 months, 9 months, 12 months, 24 months, or 48 months. In some embodiments, the stability is at 0° C., 10° C., 15° C., 20° C., 25° C., 30° C., 35° C., 37° C., and/or 40° C.

It is understood that surfactants and uptake enhancing reagents are not meant to be exclusive groups and in some cases they may be overlapping.

Additional descriptions of emulsions can be found, for example, in U.S. Pat. No. 9,352,057, the contents are herein incorporated by reference in its entirety.

### Cells and Labeling

Methods described herein may be used with a wide range of cells, including both prokaryotic and eukaryotic cells, and including mammalian cells, such as human cells. In some embodiments, the cells are mammalian cells. In some embodiments, the cells are human cells. Technologies for cell preparation include cell culture, cloning, nuclear transfer, genetic modification and encapsulation. In some embodiments, the cells are engineered cells, such as genetically engineered or genetically modified cells. In some cases, the engineered cells are recombinant human cells, e.g., a human cell expressing recombinant DNA or a recombinant protein. In some embodiments, the engineered cells are T cells comprising chimeric antigen receptors. In other words, the engineered cells are CAR-T cells.

A partial list of suitable mammalian cells includes: blood cells, myoblasts, bone marrow cells, peripheral blood cells: umbilical cord blood cells, cardiomyocytes (and precursors thereof), chondrocytes (cartilage cells), dendritic cells, fetal neural tissue, fibroblasts, hepatocytes (liver cells), islet cells of pancreas, keratinocytes (skin cells), stem cells, and diseased cells, such as cancer cells. In certain embodiments, the cells to be used are a fractionated population of immune cells. Recognized subpopulations of immune cells include lymphocytes, such as B lymphocytes (Fc receptors, MHC class II, CD19+, CD21+), hELer T lymphocytes (CD3+, CD4+, CD8−), cytolytic T lymphocytes (CD3+, CD4−, CD8+), natural killer cells (CD16+), the mononuclear phagocytes, including monocytes, neutrophils and macrophages, and dentritic cells. Other cell types that may be of interest include eosinophils and basophils.

Cells may be autologous (i.e., derived from the same individual) or syngeneic (i.e., derived from a genetically identical individual, such as a syngeneic littermate or an identical twin), although allogeneic cells (i.e., cells derived from a genetically different individual of the same species) are also contemplated. Xenogeneic (i.e., derived from a different species than the recipient) cells, such as cells from transgenic pigs, may also be administered. When the donor cells are xenogeneic, the cells can be obtained from an individual of a species within the same order, more preferably the same superfamily or family (e.g., when the recipient is a human, the cells can be derived from a primate, more preferably a member of the superfamily Hominoidea).

Cells may, where medically and ethically appropriate, be obtained from any stage of development of a donor individual (e.g., a human donor), including prenatal (e.g., embryonic or fetal), infant (e.g., from birth to approximately three years of age in humans), child (e.g., from about three years of age to about 13 years of age in humans); adolescent (e.g., from about 13 years of age to about 18 years of age in humans), young adult (e.g., front about 18 years of age to about 35 years of age in humans), adult (from about 35 years of age to about 55 years of age in humans) or elderly (e.g., from about 55 years and beyond of age in humans).

In many embodiments, cells are labeled by contacting the cells with an emulsion of the imaging compound, such that the compound is taken up (e.g., internalized) by cells. In some embodiments, cells are labeled ex vivo or in vitro under certain conditions such that the imaging compound is internalized by the cells. Both phagocytic and non-phagocytic cells may be labeled by such a method. For example, as demonstrated in WO2005072780, both dendritic cells (phagocytic) and gliosarcoma cells (non-phagocytic) can be labeled by contacting the cells with an emulsion of the imaging compound.

In some embodiments, cells at a density of about 10 million cells in about 5 ml of media are incubated overnight with 15 mg/ml of a nanoemulsion described herein. In certain embodiments, cells at a density of about 1-100 million cells in about 1-100 ml of media are incubated overnight with 5-50 mg/ml of a nanoemulsion described herein.

In certain embodiments, a method of the invention may comprise labeling cells in vivo with a ¹⁹F imaging compound and detecting labeled cells in the subject. The imaging compound can be administered to the subject, e.g., human subject, by administration routes including, but not limited to, parenterally administration, e.g., intravenous administration. The cells to be labeled may be determined by specific properties of the cells such as phagocytic activity. The cells that are labeled may be controlled by the route of administration of the imaging reagent. The types of cells that are labeled may be controlled by the nature of the imaging compound. For example, simple colloidal suspensions of imaging compound will tend to be taken up more quickly by cells with phagocytic activity. As another example, an imaging compound may be formulated with or covalently bound to a targeting moiety that facilitates selective targeting of the imaging reagent to a particular population of cells.

In some embodiments, the imaging reagent described herein is used to detect engineered cells such as CAR-T cells in a subject, e.g., a human subject. In some cases, the imaging reagent tracks CAR-T cells carrying the PFC nanoemulsion upon injection into a tumor in a subject.

In certain embodiments the cells to be labeled are stem cells and in some cases, progenitor cells. In some embodiments, the cells are pluripotent stem cells including induced pluripotent stem cells and differentiated cells thereof. Stem cell therapies are commonly used as part of an ablative regimen for treatment of cancer with high dose radiation and/or chemotherapeutic agents. Ablative regimens generally employ hematopoietic stem cells, or populations of cells containing hematopoietic stem cells and hematopoietic progenitor cells, as may be obtained, for example, from peripheral blood, umbilical cord blood or bone marrow. Cells of this type, or a portion thereof, may be labeled and tracked in vivo to monitor survival and engraftment at the appropriate location. Other types of stem cells are increasingly attractive as therapeutic agents for a wide variety of disorders. Labelled cells can be tracked or detected using any method known in the art including, but not limited to, flow cytometry, FACS, electron microscopy, magnetic resonance imaging (MRI), positron emission tomography (PET), single-photon emission coherent tomography (SPECT), ultrasonography (US), computed tomography (CT), ex vivo imaging, in vivo imaging, fluorescence microscopy and the like.

As an example, cells may be mouse embryonic stem cells, or ES cells from another model animal. The labeling of such cells may be useful in tracking the fate of such cells administered to mice, optionally as part of a preclinical research program for developing embryonic stem cell therapeutics. Examples of mouse embryonic stem cells include: the JMI ES cell line described in M. Qiu et al., Genes Dev 9, 2523 (1995), and the ROSA line described in G. Friedrich, P. Soriano, Genes Dev 5, 1513 (1991), and mouse ES cells described in U.S. Pat. No. 6,190,910. Many other mouse ES lines are available from Jackson Laboratories (Bar Harbor, Me.). Examples of human embryonic stem cells include those available through the following suppliers; Arcos Bioscience, Inc., Foster City, Calif., CyThera, Inc., San Diego, Calif., BresaGen, Inc., Athens, Ga., ES cell International, Melbourne, Australia, Geron Corporation, Menlo Park, Calif., Goteborg University, Goteborg, Sweden, Karolinska Institute, Stockholm, Sweden, Maria Biotech Co. Ltd.—Maria infertility Hospital Medical Institute, Seoul, Korea, MizMedi Hospital—Seoul National University, Seoul, Korea, National Centre for Biological; Sciences/Tata Institute of Fundamental Research, Bangalore, India, Pochon CHA University, Seoul, Korea, Reliance Life Sciences, Mumbai, India, ReNeuron, Surrey, United Kingdom, StemCells, Inc., Palo Alto, Calif., Technion University, Haifa, Israel, University of California, San Francisco, Calif., and Wisconsin Alumni Research Foundation, Madison, Wis. In addition, examples of embryonic stem cells are described in the following U.S. patents and published patent applications: U.S. Pat. Nos. 6,245,566; 6,200,806; 6,090,622; 9,351,406; 6,090,622; 5,843,780: 20020045259; 20020068045; all of which are incorporated by reference herein in their entireties. In some embodiments, the human ES cells are selected from the list of approved cell lines provided by the National Institutes of Health (NIH) and accessible at the NIH embryonic Stem Cell Registry. In certain embodiments, an embryonic stem cell line is selected from the group comprising: the WA09 line obtained from Dr. J. Thomson (Univ. of Wisconsin) and the UC01 and UC06 lines, both on the current NIH registry.

In certain embodiments, a stem cell for use in disclosed methods is a stem cell of neural or neuroendocrine origin, such as a stem cell from the central nervous system (see, for example U.S. Pat. Nos. 6,468,794; 6,040,180; 5,753,506; 5,766,948), neural crest (see, for example, U.S. Pat. Nos. 5,589,376; 5,824,489), the olfactory bulb or peripheral neural tissues (see, for example. US Patent Publication Nos. 2003/0003579; 2002/0123143; 2002/0016002 and Gritti et al. 2002 J Neurosci 22 (2):437-45), the spinal cord (see, for example, U.S. Pat. Nos. 6,361,996, 5,851,832) or a neuroendocrine lineage, such as the adrenal gland, pituitary gland or certain portions of the gut (see, for example, U.S. Pat. No. 6,171,610 and PC12 cells as described in Kimura et al., 1994, J. Biol. Chem. 269: 1896-67). In some embodiments, a neural stem cell is obtained from a peripheral tissue or an easily healed tissue, thereby providing art autologous population of cells for transplant.

Hematopoietic or mesenchymal stem cells may be employed in certain disclosed methods. Recent studies suggest that bone marrow-derived hematopoietic (HSCs) and mesenchymal stem cells (MSCs), which are readily isolated, have a broader differentiation potential than previously recognized. Purified HSCs not only give rise to all cells in blood, but can also develop into cells normally derived from endoderm, like hepatocytes (Krause et ah, 2001, Cell 105: 360-77; Lagasse et al., 2000 Nat Med 6: 1229-34). Similarly, HSCs from peripheral blood and from umbilical cord blood are expected to provide a useful spectrum of developmental potential. MSCs appear to be similarly multipotent, producing progeny that can, for example, express neural cell markers (Pittenger et al., 1999 Science 284: 143-7; Zhao et al., 2002 Exp Neurol 174: 11-20). Examples of hematopoietic stem cells include those described in U.S. Pat. Nos. 4,714,680; 5,061,620; 5,437,994; 5,914,108; 5,925,567; 5,703,197; 5,750,397; 5,716,827; 5,643,741; 5,061,620. Examples of mesenchymal stem cells include those described in U.S. Pat. Nos. 5,486,350; 5,327,735, 5,942,235; 5,972,703, those described in PCT publication nos. WO 00/53705, WO 00/02654; WO 98/20907, and those described in Pittenger et al. and Zhao et al., supra.

Stem cell lines are preferably derived from mammals, such as rodents (e.g., mouse or rat), primates (e.g., monkeys, chimpanzees or humans), pigs, and ruminants (e.g., cows, sheep and goats), and particularly from humans. In certain embodiments, stem cells are derived from an autologous source or an HLA-type matched source. For example, stem cells may be obtained from a subject in need of pancreatic hormone-producing cells (e.g., diabetic patients in need of insulin-producing cells) and cultured to generate autologous insulin-producing cells. Other sources of stem cells are easily obtained from a subject, such as stem cells from muscle tissue, stem cells from skin (dermis or epidermis), stem cells from fat, and stem cells from any organ or tissue of the body.

In some embodiments, cells for administration to a human should be compliant with good tissue practice guidelines set by the U.S. Food and Drug Administration (FDA) or equivalent regulatory agency in another country. Methods to develop such a cell line may include donor testing, and avoidance of exposure to non-human cells and products.

Cells derived from a donor (optionally the patient is the donor) may be administered as unfractionated or fractionated cells, as dictated by the purpose of the cells to be delivered. Cells may be fractionated to enrich for certain cell types prior to administration. Methods of fractionation are well known in the art, and generally involve both positive selection (i.e., retention of cells based on a particular property) and negative selection (i.e., elimination of cells based on a particular property). As will be apparent to one of skill in the art, the particular properties (e.g., surface markers) that are used for positive and negative selection will depend on the desired population of cells. Methods used for selection/enrichment of cells may include immunoaffinity technology or density centrifugation methods. Immunoaffinity technology may take a variety of forms, as is well known in the art, but generally utilizes an antibody or antibody derivative in combination with some type of segregation technology. The segregation technology generally results in physical segregation of cells bound by the antibody and cells not bound by the antibody, although in some instances the segregation technology which kills the cells bound by the antibody may be used for negative selection.

Any suitable immunoaffinity technology may be utilized for selection/enrichment of the selected cells to be used, including fluorescence-activated cell sorting (FACS), panning, immunomagnetic separation, immunoaffinity chromatography, antibody-mediated complement fixation, immunotoxin, density gradient segregation, and the like. After processing in the immunoaffinity process, the desired cells (the cells bound by the immunoaffinity reagent in the case of positive selection, and cells not bound by the immunoaffinity reagent in the case of negative selection) are collected and either subjected to further rounds of immunoaffinity selection/enrichment, or reserved for administration to the patient.

Immunoaffinity selection/enrichment is typically carried out by incubating a preparation of cells comprising the desired cell type with an antibody or antibody-derived affinity reagent (e.g., an antibody specific for a given surface marker), then utilizing the bound affinity reagent to select either for or against the cells to which the antibody is bound. The selection process generally involves a physical separation, such as can be accomplished by directing droplets containing single cells into different containers depending on the presence or absence of bound affinity reagent (FACS), by utilizing an antibody bound (directly or indirectly) to a solid phase substrate (panning, immunoaffinity chromatography), or by utilizing a magnetic field to collect the cells which are bound to magnetic droplets via the affinity reagent (immunomagnetic separation). Alternately, undesirable cells may be eliminated from the preparation using an affinity reagent which directs a cytotoxic insult to the cells bound by the affinity reagent. The cytotoxic insult may be activated by the affinity reagent (e.g., complement fixation), or may be localized to the target cells by the affinity reagent (e.g., immunotoxin, such as ricin B chain).

Although it is expected that methods disclosed herein will be frequently used for in vivo monitoring of cells, it should be noted that the methodologies are equally effective for the monitoring of cells in culture (i.e., in vitro), in a tissue sample or other ex vivo cellular material. For therapeutic uses, cells may be labeled at a desired step during the preparation for administration to the patient.

A variety of methods may be used to label cells with imaging reagent. In general, cells will be placed in contact with imaging reagent such that the imaging reagent becomes associated with the cell. Conditions will often be standard cell culture conditions designed to maintain, cell viability. The term “associated” is intended to encompass any manner by which the imaging reagent and cell remain in sufficiently close physical proximity for a sufficient amount of time as to allow the imaging reagent to provide useful information about the position of the cell, whether in vivo or in vitro. Imaging reagent may be located intracellularly, e.g. after phagocytosis or surfactant mediated entry into the cell. Immune cells, such as dendritic cells, macrophages and T cells are often highly phagocytic and data presented herein and in other studies demonstrate that such cells, and other phagocytic cell types, are readily labeled. Other cell types, such as stem cells may also be labeled, regardless of phagocytic activity. Imaging reagent may be inserted into a cell membrane or covalently or non-covalently bound to an extracellular component of the cell. For example, certain linear fluorocarbons described herein may be derivatized to attach one or more targeting moiety. A targeting moiety will be selected to facilitate association of the imaging reagent with the cell to be labeled. A targeting moiety may be designed to cause non-specific insertion of the fibrocarbon into a cell membrane (e.g., a hydrophobic amino acid sequence or other hydrophobic moiety such as a palmitoyl moiety or myristoyl moiety) or to facilitate non-specific entry into the cell. A targeting moiety may bind to a cell surface component, as in the case of receptor ligands. A targeting moiety may be a member of a specific binding pair, where the partner is a cell surface component. The targeting moiety may be, for example, a ligand for a receptor, or an antibody, such as a monoclonal or polyclonal antibody or any of the various polypeptide binding agents comprising a variable portion of an immunoglobulin (e.g., Fv fragment, single chain Fv (scFv) fragment, Fab′ fragment, F(ab′)2 fragment, single domain antibody, camelized antibody, humanized antibody, diabodies, tribodies, tetrabodies). In certain embodiments, the fluorocarbon imaging reagent comprises perfluoro-15-crown ether.

Cellular labeling with fluorocarbons emulsions can also be facilitated using transfection agents to aid in cell delivery. Often transfection agents consist of cationic lipids, cationic liposomes, poly-cations, and the like. The transfection agent is pre-mixed with the fluorocarbon emulsion labeling agent, whereby it becomes associated with, or coats, the emulsion droplets. The transfection agent-treated emulsion droplets are then added to the cultured cells and incubated so that the cells become labeled. Common transaction agents include Lipofectamine (Invitrogen, Inc) FuGene, DOTAP (Roche Diagnostics, Inc.), and poly-L-lysine. Small proteins can also be used as transfection agents, such as many types of protamines. Protamines, the major DNA-landing proteins in the nucleus of sperm in most vertebrates, package the DNA in a volume less than 5% of a somatic cell nucleus. Protamines are simple proteins of low molecular weight that are rich in arginine and strongly basic. Commercially available protamines come from the sperm of salmon and certain other species of fish. The term “protamine” as used herein, refers to a low molecular weight cationic, arginine-rich polypeptide. The protamine molecule typically comprises about 20 to about 200 amino acids and is generally characterized by containing at least 20%, 50% or 70% arginine. Protamines are often formulated as salts, with one or more counter ions such as sulfate, phosphate and chloride.

Data provided in this application show that protamines (e.g., protamine sulfate) are highly effective in delivering PFPE fluorocarbon emulsion droplets to cultured cells. Suitable protamine sulfates can come from a variety of sources (e.g., salmon, herring, trout, etc.) and be of various grades and forms (e.g., USP, grades II, III, X, etc.), with and without histones or any recombinant derivative. Examples of other protamine solutions that may be used as transfection agents include protamine phosphate, protamine chloride, protamine sulfate-2, protamine sulfate-3, protamine sulfate-10, and protamine free base.

Data provided in this application shows self deliverable nanoemulsions prepared with fluorocarbon imaging reagents (e.g., perfluoro-15-crown-5 ether or PFPE oxide) and incorporate a Plutonic™ surfactant, optionally with Protamine Sulfate, or Cremophor EL® with an emulsifier and an additive. Simple co-incubation of cells with certain self-deliverable nanoemulsions provides sufficient cell labeling for imaging, without the need for transfection reagents.

Where cells are to be used in a therapeutic regimen, various methods have been used for delivery of cells including injections and use of special devices to implant cells in various organs. The present invention is not tied to any particular delivery method. Labeled cells may be monitored regardless of whether the cells are delivered directly to a particular site or delivered systemically. For example, labeled dendritic cells were successfully imaged following either a focal implantation directly into tissues or an intravenous injection, and T-cells were imaged following intraperitoneal injection. Cells may be inserted into a delivery device which facilitates introduction by injection or implantation into the subjects. Such delivery devices may include tubes, e.g., catheters, for injecting cells and fluids into the body of a recipient subject. In some embodiments, the tubes additionally have a needle, e.g., a syringe, through which the cells of the disclosure can be introduced into the subject at a desired location. The cells may be prepared for delivery in a variety of different forms. For example, the cells may be suspended in a solution or gel or embedded in a support matrix when contained in such a delivery device. Cells may be mixed with a pharmaceutically acceptable carrier or diluent in which the cells of the disclosure remain viable. Pharmaceutically acceptable carriers and diluents include saline, aqueous buffer solutions, solvents and/or dispersion media. The use of such earners and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the disclosure may be prepared by Incorporating cells as described herein in a pharmaceutically acceptable carrier or diluent and, as required, other ingredients enumerated above, followed by filtered sterilization.

Additional descriptions of useful cells and methods of labeling such cells can be found, for example, in U.S. Pat. No. 9,352,057, the contents of which is herein incorporated by reference in its entirety.

In some embodiments, cells are labeled with any nanoemulsion described herein by contacted or incubating the cells with about 1 mg/ml to about 50 mg/ml (e.g., about 1 mg/ml-about 50 mg/ml, about 5 mg/ml-about 50 mg/ml, about 5 mg/ml-about 45 mg/ml, about 5 mg/ml-about 40 mg/ml, about 5 mg/ml-about 35 mg/ml, about 5 mg/ml-about 30 mg/ml, about 5 mg/ml-about 20 mg/ml, about 15 mg/ml-about 50 mg/ml, about 15 mg/ml-about 40 mg/ml, about 15 mg/ml-about 30 mg/ml, about 10 mg/ml-about 50 mg/ml, about 1 mg/ml, about 2 mg/ml, about 5 mg/ml, about 8 mg/ml, about 10 mg/ml, about 12 mg/ml, about 15 mg/ml, about 17 mg/ml, about 20 mg/ml, about 22 mg/ml, about 25 mg/ml, about 27 mg/ml, about 30 mg/ml, about 32 mg/ml, about 35 mg/ml, about 37 mg/ml, about 40 mg/ml, about 42 mg/ml, about 45 mg/ml, about 47 mg/ml, and about 50 mg/ml) of the nanoemulsion.

### Nuclear Magnetic Resonance Imaging Techniques

As described herein, also referred to herein as a type of imaging modality, nuclear magnetic resonance techniques may be used to detect populations of labeled cells. The term “detect” is used to include any effort to ascertain the presence or absence of a labeled molecule or cell, particularly by a nuclear magnetic resonance technique. The term “detect” is also intended to include more sophisticated measurements, including quantitative measurements and two- or three-dimensional image generation. For example, MRI may be used to generate images of such cells. In many instances, the labeled cells may be administered to a living subject. Following administration of the cells, some portion of the subject, or the entire subject, may be examined by MRI to generate an MRI data set. In other instances, the emulsion is injected directly iv, and the subject is subsequently imaged at one or more time points. A “data set”, as the term is used herein, is intended to include raw data gathered during magnetic resonance probing of the subject material, the acquisition parameters, as well as information processed, transformed or extracted from the raw data. The raw data includes transient signals obtained by MRI (magnetic resonance imaging)/MRS (magnetic resonance spectroscopy), including the free-induction decays, spin-echoes, stimulated-echoes, and/or gradient echoes. Examples of processed information include two-dimensional or three-dimensional pictorial representations of the subject material. The processed information may also include magnitude images, the real and imaginary image components, as well as the associated phase map images. Another example of extracted information is a score representing the amount or concentration of imaging reagent or ¹⁹F signal in the subject material. By using the amount of ¹⁹F signal in the subject material, and a calibration of the mean amount of imaging reagent per cell pre-implantation (m the case of ex vivo labeling), one can estimate the absolute number of cells in the subject material. The amount of ¹⁹F signal present in a subject material can be represented or calculated in many ways; for example, the average signal-to-noise-ratio (SNR) of the ¹⁹F signal for a region of interest (ROI) may be measured and used to calculate the abundance of labeled cells. In certain embodiments, the average intensity, or pixel- or voxel-wise summation of the ¹⁹F signal may be used to calculate the abundance of labeled cells. This type of data may be gathered at a single region of the subject, such as, for example, the spleen or another organ of particular relevance to the labeled cells. Labeled cells may be examined in contexts other than in the subject. It may be desirable to examine labeled cells in culture. In certain embodiments, labeled cells may be applied to or generated within a tissue sample or tissue culture, and labeled cells may therefore be imaged in those contexts as well. For example, an organ, tissue or other cellular material to be transplanted may be contacted with an imaging reagent to generate labeled cells prior to implantation of such transplant in a subject.

In general, labeling agents of the disclosure are designed for use in conventional MRI detection systems. In the most common implementation of MRI, one observes the hydrogen nucleus (proton, ¹H) in molecules of mobile water contained in subject materials. To detect labels disclosed herein, an alternate nucleus is detected, ¹⁹F. ¹⁹F MRI has only slightly less intrinsic sensitivity compared to ¹H; the relative sensitivity is approximately 0.83. Both have a nuclear spin of +½. The natural isotopic abundance of ¹⁹F is 100%, which is comparable to 99.985% for ₁H. The physical principles behind the detection and image formation are the same for both ¹H and ¹⁹F MRI. The subject material is placed in a large static magnetic field. The field tends to align the magnetic moment associated with the ¹H or ¹⁹F nuclei along the field direction. The nuclei are perturbed from equilibrium by pulsed radio-frequency (RF) radiation at the Larmor frequency, which is a characteristic frequency proportional to the magnetic field strength where nuclei resonantly absorb energy. Upon removing the RF, the nuclei induce a transient voltage in a receiver antenna; this transient voltage constitutes the nuclear magnetic resonance (NMR) signal. Spatial information is encoded in both the frequency and/or phase of the NMR signal by selective application of magnetic field gradients that are superimposed onto the large static field. The transient voltages are generally digitized, and then these signals may be processed by, for example, using a computer to yield images.

At constant magnetic field strength, the Larmor frequency of ¹⁹F is only slightly lower (about 6%) compared to ¹H. Thus, it is straightforward to adapt conventional MRI scanners, both hardware and software, to acquire ¹⁹F data. The ¹⁹F detection may be coupled with different types of magnetic resonance scans, such as MRI, MRS or other techniques. Typically, it will be desirable to obtain a ¹H MRI image to compare against the ¹⁹F image. In a living organism or other biological tissue, the proton MRI will provide an image of the subject material and allow one to define the anatomical context of the labeled cells detected in the ¹⁹F image. In some embodiments, data is collected for both ¹⁹F and ¹H during the same session; the subject is not moved during these acquisitions to better ensure that the two data sets are in spatial registration. Normally, ¹⁹F and ¹H data sets are acquired sequentially, in either order. An RF coil (i.e., antenna) can be constructed that can be electrically tuned from the ¹⁹F and ¹H Larmor frequency. Tuning between these two frequencies can be performed manually (e.g. via an electro-mechanical variable capacitor or inductor), or electrically, via active electronic circuitry. Alternatively, with appropriate modifications to the hardware and/or software of the MRI instrument, both data sets can be acquired simultaneously, for example, to conserve imaging time. Simultaneous acquisition of the ¹⁹F and ¹H data sets require an RF coil or antenna that can be electrically tuned simultaneously to the ¹⁹F and ¹H Larmor frequency (i.e., a double-tuned coil). Alternatively the RF coil can be “broadband,” with one broadly-tuned electrical resonance that covers both Larmor frequencies (i.e., ¹⁹F and ¹H). Other imaging techniques, such as fluorescence detection may be coupled with ¹⁹F MRI. This will be particularly desirable where a fluorocarbon imaging reagent has been derivatized with a fluorescent moiety. In other embodiments, the ¹⁹F MRI scan may be combined with a PET scan in the same subject or patient by using dual-model radioactive ¹⁸F/¹⁹F fluorocarbon labeling reagents as described herein.

MRI examination may be conducted according to any suitable methodology known in the art. Many different types of MRI pulse sequences, or the set of instructions used by the MRI apparatus to orchestrate data collection, and signal processing techniques (e.g., Fourier transform and projection reconstruction) have been developed over the years for collecting and processing image data (for example, see Magnetic Resonance Imaging, Third Edition, editors D. D. Stark and W. G. Bradley, Mosby, Inc., St. Louis Mo. 1999). The reagents and methods of this disclosure are not tied to any particular imaging pulse sequence or processing method of the raw NMR signals. For example, MRI methods that can be applied to this disclosure broadly encompasses spin-echo, stimulated-echo, gradient-echo, free-induction decay based imaging, and any combination thereof. Fast imaging techniques, where more than one line in k-space or large segments of k-space are acquired from each excited signal, are also highly suitable to acquire the ¹⁹F (or ¹H) data. Examples of fast imaging techniques include fast spin-echo approaches (e.g., FSE, turbo SE, TSE, RARE, or HASTE), echo-planar imaging (EPI), combined gradient-echo and spin-echo techniques (e.g., GRASE), spiral imaging, and burst imaging. The development of new and improved pulse sequence and signal processing methods is a continuously evolving field, and persons skilled in the art can devise multiple ways to image the ¹⁹F labeled cells in their anatomical context.

As another example of a nuclear magnetic resonance technique, MRS can be used to detect the presence of fluorocarbon-labeled cells in localised tissues or organs. Normally MRS methods are implemented on a conventional MRI scanner. Often the localized volume of interest (VOI) is defined within a conventional anatomical ¹H MRI scan. Subsequently, the magnitude of the ¹⁹F NMR signal observed within the VOI is directly related to the number of labeled cells, and/or the mean concentration of PFPE per cell present in the tissue or organ. Methods for isolating a VOI within a much larger subject are well known the art (for example, Magnetic Resonance Imaging, Third Edition, Chapter 9, Editors D. D. Stark and W. G. Bradley, Mosby, Inc., St Louis Mo. 1999). Examples include using a localised RF surface coil near the VOI, surface spoiling, surface coil Bi-gradient methods, slice-selective Bo-gradient techniques, STEAM, PRESS, image selective in vivo spectroscopy (ISIS), and magnetic resonance spectroscopic imaging (MRSI).

The development of new and improved pulse sequence and signal processing methods is continuously evolving for MRS, and persons skilled in the art can devise multiple ways to detect the ¹⁹F NMR signals emanating from the fluorocarbon labeled cells in VOIs.

In some embodiments, the subject material is a fixed or otherwise preserved specimen of tissue that has been biopsied or necropsied from the animal or human. The subject material is then subjected to conventional high-resolution, one or multi-dimensional, liquid state ¹⁹F NMR to determine the amount of fluorine present in the sample. The fluorine content is directly related to the number of labeled cells in the subject material specimen. In the case of in situ labeling of resident phagocytes (e.g., monocytes, macrophage, neutrophil, cells of the liver) with fluorine emulsion as described above (e.g., using nanoemulsion 3), the amount of ¹⁹F measured in the sample is directly proportional to the number of these phagocytes present in the tissue. In this way one can assay the relative amount of inflammation in the intact tissues without having to use histology or any other destructive and time-consuming techniques. In certain embodiments, to analyze the ¹⁹F content of the tissue, one uses one-dimension ¹⁹F NMR. In certain embodiments, a ¹⁹F reference compound will be added to the sample of known number of ¹⁹F spins that has a chemical shift that is different than the composition of the cell labeling emulsion (see below). In certain embodiments, the relative integrated areas under the emulsion peak and reference peak can be used to calculate the absolute number of fluorines present in the tissue sample. In certain embodiments, the weight of the tissue sample can also be incorporated into the calculation to extract the mean fluorine density of the tissue sample, and this parameter can be considered a quantitative index of inflammation or “inflammation index”.

In certain embodiments the disclosure provides a method of quantifying the numbers of labeled cells in vivo or in subject materials within an ROI. An ROI may include all labeled cells in a subject or labeled cells in specific organs such as the pancreas, specific tissues such as lymph nodes, or any region or of one or more voxels showing detectable MRI/MRS ¹⁹F signal. A ROI can be an otherwise undefined area beyond a particular experiment. There are a number of ways that labeled cells may be quantified in the subject materials or in vivo, as described herein.

In the case or ex vivo labeling, calibrating the mean “cellular dose” of ¹⁹F labeling agent pre-implantation of a particular cell population is often a pre-requisite for quantitative cell determinations in subject materials or the patient. It is anticipated that different cell types have different inmate abilities to take up the labeling agents in vitro, and thus the cellular dose of the labeling agent will also vary. Furthermore, different cells of the same type acquired from different sources (e.g., different patients) may have different affinities for the labeling agent. Thus a cellular dose calibration may be required. This calibration may be used, initially, to modify the labeling protocol (i.e., incubation conditions, duration of time that cells are incubated with labeling fluorocarbon emulsion, concentration of fluorocarbon emulsion in culture medium during labeling, etc.) to achieve a certain range of cellular dose before labeled cells are actually used in a subject to be imaged. Alternatively, one can fix the labeling conditions and protocol and measure the mean value ¹⁹F labeled per cell, as is, for subsequent quantification in the subject to be imaged. In certain embodiments the mean number of ¹⁹F molecules (F's) per cell of a labeled cell population is measured (i.e., calibrated) in vitro prior to administration of the cells to the subject or patient. In certain embodiments the mean number of ¹⁹F molecules (F's) per cell of a labeled cell population is calibrated in a test population of cells of a particular type, not necessarily destined for a patient, but used to calibrate cellular dose of labeling agent as a consequence of a particular labeling protocol or set of conditions; optionally, the value of cellular dose is then used for future labeling and in vivo imaging experiments in the same population type of cells with the same labeling protocol.

The cellular dose of labeling agent can be assayed in vitro using a variety of quantitative techniques. For example, one can use a one-dimensional (1D) ¹⁹F NMR spectrum obtained from a cell pellet, cell suspension, or cell lysate, of a known number of labeled cells. From this spectrum, one can calculate the integrated area of the ¹⁹F spectrum or a portion thereof, originating from the labeling reagent associated with the cells. The integrated area of the ¹⁹F spectrum, denoted S_{cells}, is directly proportional to the total amount of ¹⁹F in the cell pellet, suspension, or lysate. To measure the absolute number of ¹⁹F nuclei, the measured S.sub.cells may be normalized to a ¹⁹F standard. A ¹⁹F standard can be, for example, a solution of a known volume and concentration of a fluoro-chemical, where one can calculate the total number of ¹⁹F nuclei in the standard, denoted F_{scan}. A suitable fluoro-chemical reference ideally has a simple ¹⁹F NMR spectrum, preferable with a single narrow resonance (e.g. trifluoroacetic acid or TFA) and optionally a ¹⁹F chemical shift that is significantly different than the labeling fluorocarbon. The ¹⁹F standard can be placed in the same NMR tube as the labeled cell material being measured, in a separate tube, or optionally can be measured in a separate experiment using the same NMR instrument. The integrated area of the spectrum from the ¹⁹F standard, denoted Sₛₜₐₙ, can then be measured. Subsequently, the mean number of ¹⁹F per labeled cell, denoted F_{c}, can be calculated, for example using the following formula:

$${F}_{c} = \frac{{S}_{cells}}{{S}_{s t a n}} {F}_{stan} \frac{1}{{N}_{cells}}$$

where N_{cells} is the number of labeled cells contained in the in vitro test sample. Quantitative NMR methods for ¹⁹F and other nuclei are well known in the art, and those skilled can devise many variations to the cellular dose calibration procedure described above. Besides ¹⁹F NMR, there are other quantitative methods that can be used to assay the cellular dose of the labeling reagent. For example, a reagent may be labeled fluorescently, luminescently, optically, or radioactively (see, U.S. Patent Publication Nos. 2007/0258886 and 2013/0343999, herein incorporated by reference in their entireties).

Similarly, in the case of in situ cell labeling of circulating phagocytes following iv injection of emulsion, to measure the effective cell labeling, one can extravesate a portion of peripheral blood from the subject and measure the effective cell loading of leukocytes using the methods described above. Furthermore, one or more of the various cell sorting or enrichment techniques can be used to sort out phagocytic cells (e.g., macrophages) prior to the loading measurement (above) to better define which cell population has been labeled in situ. The measured cell labeling parameter can then be used to calculate the apparent number of inflammatory cells present in tissue using the magnetic resonance methods described herein.

In order to extract accurate quantification of labeled cells and/or relative inflammation score from the ¹⁹F MRI/MRS data sets, additional calibrations and standards may be employed. For example, one can use a calibrated external ¹⁹F reference (i.e., phantom) during the actual ¹⁹F MRI/MRS scan of the subject material containing labeled cells. The image intensity of the calibrated phantom is used, tor examples, when analyzing the ¹⁹F MRI/MRS data set to prove an absolute standard for the number of ¹⁹F nuclei when examining the subject material or patient. The calibrated phantom is used to normalize the sensitivity of the particular MRI/MRS system that has been loaded with a particular subject to be imaged. The ¹⁹F reference may be, for example, one or more vessels containing a solution of a known concentration of ¹⁹F nuclei. In some embodiments, the solution contains a dilute concentration of the emulsified fluorocarbon labeling reagent. Optionally, the solution contains non-emulsified fluorocarbon labeling reagent, a gel, or liquid, for example that has been diluted in a suitable solvent. Optionally, the solution can be composed of another fluoro-chemical, ideally wish a simple ¹⁹F NMR spectrum, preferably with a single narrow NMR resonance (e.g. trifluoroacetic acid (TFA) or trifluoroacetamide (TFM) and other fluorinated acids, trifluorotoluene or trifluoroethanol). In some embodiments, the T1 and T2 values of the reference solution are similar to those of the labeling reagent. Optionally, the solution can contain perfluorocarbon-labeled cells, or lysines of the same. The non-cellular reference has the advantage of longer storage times. Optionally, the solution can take the form of a gel. The vessel containing the solution can be sealable, and can take a variety of geometries; vessel geometries including ellipsoidal, cylindrical, spherical, and parallel piped shapes. One or more vessels containing ¹⁹F reference solution can be used during the ¹⁹F MRI/IRS of the subject material if multiple ¹⁹F references (i.e., vessels) are used they can contain the same ¹⁹F concentration or different concentrations, and in the case of the latter, they ideally contain graded concentrations of fluorochemical. The placement of the calibrated ¹⁹F reference vessel(s) can in some embodiments, be placed externally or alongside, or optionally inside, the imaged subject or patient prior to data acquisition. In some embodiments, the reference is imaged using ¹⁹F MRI along with the subject in the same image field of view (FOV). Optionally, ¹⁹F MRS data is acquired in the reference either sequentially or in parallel with the subject data set. Optionally, data from the reference can be acquired using MRI/MRS acquired in a separate scan. Optionally, the external reference is not scanned along with a subject in every ¹⁹F MRI/MRS examination, but rather, values of the reference ¹⁹F signal intensity acquired using MRI/MRS is used from a scan of a comparable subject or a simulated-subject. In a given ¹⁹F MRI/NRS scan, the calibrated ¹⁹F standard may be sampled by one or more voxels. The observable ¹⁹F intensity produced by a voxel may be proportional to the concentration of the fluorochemical in the solution for gel and the voxel volume. Often in a ¹⁹F MRI scan the reference standard is comprised of many voxels. Often one calculates the mean intensity of one, several, or all voxels in the reference standard. Optionally, the mean image intensity is calculated over an ROI defined with in the ¹⁹F image of the reference standard. Optionally, the physical geometry of the reference standard vessel contributes to defining the observed ¹⁹F signal intensity, for example, the volume compartment(s) containing the ¹⁹F reference solution is smaller than the voxel volume. In other embodiments, the calibrated external reference relies on a solution with a ¹H signal intensity of a known number of detectable ¹H; in this case the sensitivity of the ¹⁹F signal in the subject material is reference to a ¹H calibrated standard. Ideally the solution or gel in the ¹H calibrated reference (contained in a vessel as described above) yields a simple ¹H NMR spectrum, preferably with a single narrow NMR resonance (e.g., H₂O, or mixtures of H₂O—D₂O). Other than a different nuclei, the use of the ¹H standard reference is the same in many other respects as described above for the ¹⁹F reference. Optionally, the calibrated reference standard contains any other MRI/MRS-active nuclei. In some embodiment, the reference is an internal organ or tissue detected via ¹H MRI/MRS, where the data may be raw or normalized. In other embodiments, the reference is a standard that is not scanned with the subject, but is calibrated by relevant factors such as the weight of the patient or the size of the body cavity.

By computationally manipulating or combining two or more key parameters from the ¹⁹F MRI/MRS data set, one can calculate the number of labeled cells and/or relative amount of inflammation present in an ROI as described herein. For example, a fey set of parameters may include: (i) the cellular dose of labeling agent (i.e., Fe) measured in vitro; (ii) in vivo ¹⁹F MRI/MRS data set taken in the subject at one or more time points following labeled cell administration; (iii) the voxel volume; (iv) the in-plane voxel area (i.e., area of the image pixel); (v) optionally, the MRI/MRS data set from the ¹⁹F reference standard; (vi) optionally, the measured Johnson noise of the ¹⁹F MRI/MRS data in the subject material; (vii) optionally, the measured signal-to-noise ratio (SNR) of one or more voxels of the ¹⁹F MRI/MRS data set in the subject material; (viii) optionally, the measured SNR of one or more voxels of the ¹⁹F MRI/MRS data set from the reference standard; (ix) optionally, the ¹⁹F NMR relaxation times (T1, T2, and T2*) of the subject material; (x) optionally, the ¹⁹F NMR relaxation times (T1, T2, and T2*) of the reference standard (for example, see Magnetic Resonance Imaging, Third Edition, chapter 4, editors D. D. Stark and W. G. Bradley, Mosby, Inc., St, Louis Mo. 1999). Those skilled in the art can derive other parameters, combinations of the above set, or derivations thereof, particularly from the ¹⁹MRI/MRS dataset, that can be used to quantify the number of labeled cells in situ. In certain embodiments the above set of key parameters can be used to derive quantitative or statistical measures of the accuracy or confidence of the measured number of labeled cells.

There are many ways to combine the key parameters, (i-x, above), any subsets of these, or any of their combinations or approximations, to estimate the effective number of labeled cells seen by ¹⁹F MRI in the subject material, denoted by N_{c}. For example, one can use an equation of the following form:

$${N}_{c} = \frac{[ {F}_{R} ] v}{{I}_{R}} \frac{1}{{F}_{c}} {\sum }_{{F}_{c}}^{{N}_{ROI}} {I}_{c}^{( i )}$$

where: N_{c}=total number of labeled cells in the ROI; [F_{R}]=concentration of ¹⁹F in the calibrated ¹⁹F reference solution (or gel); v=voxel volume; I_{R}=mean intensify of the calibrated ¹⁹F reference taken with the MRI/MRS scan, averaged over one or more voxels, F_{c}=average ¹⁹F cellular dose of the labeling agent measured in vitro; N_{ROI}=number of voxels in the ROI containing labeled cells; I_{c}⁽ⁱ⁾=image intensify of the i^{th} voxel in the ROI containing labeled cells; i=unitless index for voxels in the ROI containing labeled cells. See, U.S. Patent Publication No. 2013/0343999, herein incorporated by reference in its entirety.

There are also many ways to approximate N_{c} from the ¹⁹F data set. For example, one could use the following expression.

$${N}_{c} \approx \frac{{I}_{c}^{a v g}}{{I}_{R}} [ {F}_{R} ] v \frac{1}{{F}_{c}} {N}_{ROI}$$

where I_{c}^{avg} is the average intensity of the ROI containing the labeled cells, (i.e. the average intensity of the N_{ROI} voxels).

As another example, one could use the following expression.

$${N}_{c} \approx \frac{{I}_{c}^{a v g}}{{I}_{R}} {V}_{c} \frac{1}{{F}_{c}} [ {F}_{R} ]$$

where V_{c} is the total volume of the ROI containing the labeled cells.

As a further example, one could use the following expression.

$${N}_{c} \approx \frac{{I}_{c}^{a v g}}{{I}_{R}} \frac{{V}_{c}}{{V}_{{r}_{c}}} \frac{1}{{F}_{c}} {N}_{R}$$

where V_{R} is the effective volume of the reference in the ¹⁹F MRI/MRS and N_{R} is the number ¹⁹F nuclei in V_{R}. Note that in all of the above formulas the various intensities (i.e., I_{R}, I_{c}^{avg}, I_{c}⁽ⁱ⁾) can be normalized to the image noise, and thus the above formulas can be equivalently expressed in terms of the appropriate SNR values for the particular regions. Thus, there are many ways to estimate the number of labeled cells, N_{c}, and many similar forms of these basic expressions can be derived by basic mathematical manipulations, however, all rely on the same basic content contained within the input parameters described by (i-x). Furthermore, quantification of labeled cells in an ROI need not be expressed in terms of absolute numbers or effective cell numbers. Other quantitative indices can be derived that are indicative of the amount of cells in an ROI. For example, one can calculate the ratio I_{c}^{avg}/I_{R}, or the ratio of the average SNR values observed in the ROI and the reference; all of these fall within subsets of the above expressions and/or the parameters. See, U.S. Patent Publication No. 2013/0343999, herein incorporated by reference in its entirety.

It is noted that the above analysis of cell numbers and related indices assume that the ¹⁹F NMR relaxation times (i.e., particularly T1 and/or T2) of the fluorocarbon label is approximately the same as material in the calibrated ¹⁹F reference standard. In the case that the relaxation times are not comparable, one of skill in the art can readily correct for this by employing the known MRI intensity equations of the particular imaging protocol being used, expressed in terms of T1 and T2.

Optionally, the ¹⁹F MRI data set of the subject material can undergo post-processing before the actual cell quantification calculation is performed (as described above). For example, post-processing algorithms may include “de-noising” the ¹⁹F data set. This can be accomplished by, for example, by thresholding the image to cut off low-intensity noise; this involves rescaling the image intensity so that low values are set to zero. In magnitude MRI images, random Johnson noise is often apparent and uniformly distributed across the image FOV. It is well known in the art that one can threshold out the low-level image intensity so that regions known to contain no true signal (i.e. devoid of ¹⁹F and/or ¹H nuclei) appear to have a null or very near-null intensity. This process can be performed in an ad-hoc fashion (i.e., “manually” or by visual inspection), or by using a computer algorithm. In other embodiments, de-noising of the data set can be achieved by using other algorithms, for example using wavelet analysis, and many methods are known in the art for image de-noising.

The following references are incorporated in their entirety herein: Khare, A., et al., INTERNATIONAL JOURNAL OF WAVELETS MULTIRESOLUTION AND INFORMATION PROCESSING, 3 (4): 477-406 December 2005; Cruz-Enriquez, H., et al., IMAGE ANALYSIS AND RECOGNITION, 3656: 247-254 2005; Awate, S P., et al., INFORMATION PROCESSING IN MEDICAL IMAGING PROCEEDINGS, 3565: 677-688 7005; Ganesan. R.; et al., IIE TRANSACTIONS, 36 (9): 787-806 September 2004; Seheunders, P., IEEE TRANSACTIONS ON IMAGE PROCESSING, 13 (4): 475-485 April 2004; Ghugre, N R., MAGNETIC RESONANCE IMAGING, 21 (8): 913-921 October 2003; Bao, P., et al., IEEE TRANSACTIONS ON MEDICAL IMAGING, 22 (9): 1089-1099 September 2003; Wu, Z Q., et al., ELECTRONICS LETTERS, 39 (7): 603-605 Apr. 3, 2003; LaConte, S M., et al., MAGNETIC RESONANCE IN MEDICINE, 44 (5): 746-757 November 2000: Laine, A F., ANNUAL REVIEW OF BIOMEDICAL ENGINEERING, 2: 511-550 2000; Zuroubi, S., et al., MAGNETIC RESONANCE IMAGING, 18 (1): 59-68 January 2000: Nowak, RD., IEEE TRANSACTIONS ON IMAGE PROCESSING, 8 (10): 1408-1419 October 1999; and Healy, D M., et al., ANNALS OF BIOMEDICAL ENGINEERING, 23 (5): 637-665 September-October 1995.

Other types of post-processing algorithms are known in the art that can be applied to the ¹⁹F MRI data set before or after quantification, such as zero-filing (A Handbook of Nuclear Magnetic Resonance, 2nd Edition, Ray Freeman, Addison Wesley Longman Press 1997) and various image interpolation, de-noising, and image smoothing algorithms (for example, see The Image Processing Handbook, 3rd Edition, John C. Russ, CRC Press/IEEE Press).

In certain embodiments the above set of key parameters (i-x) can be used to derive quantitative or statistical measures of the accuracy or confidence of the measured number of labeled cells or related indices. ¹⁹F MRI/MRS data sets are often subject to SNR limitations within ROI, and thus if is often useful to calculate a metric of the confidence or accuracy of the measurement. Many methods are known in the art for the statistical analysis of MRI and other biomedical-type images. It is understood that some embodiments described herein encompass these known methods.

Additional descriptions of useful MRI techniques and the like can be found, for example, in U.S. Pat. No. 9,352,057, the contents are herein incorporated by reference in its entirety.

### Pharmaceutical Formulations and Uses

Methods of administration of the emulsions of the application are well-known to those of skill in the art. To achieve the desired activity, the emulsions can be administered in a variety of unit dosage forms. The dose will vary according to the particular emulsion. The dose will also vary depending on the manner of administration, the overall health, condition, size, and age of the patient.

In certain embodiments, administration of the emulsions may be performed by an intravascular route, e.g., via intravenous infusion by injection. In certain embodiments, other routes of administration may be used. Formulations suitable for injection are found in Remington's Pharmaceutical Sciences, Mack Publishing Company, Philadelphia, Pa., 17th ed. (1983). Such formulations must be sterile and non-pyrogenic, and generally will include a pharmaceutically effective carrier, such as saline, buffered (e.g., phosphate buffered) saline, Hank's solution, Ringer's solution, dextrose/saline, glucose solutions, and the like. The formulations may contain pharmaceutically acceptable auxiliary substances as required, such as, tonicity adjusting agents, wetting agents, bactericidal agents, preservatives, stabilizers, and the like. In certain embodiments suitable buffers for intravenous administration are used to aid in emulsion stability. In certain embodiments glycols are used to aid in emulsion stability.

In certain embodiments, administration of the emulsions may be performed by a parenteral route, typically via injection such as intra-articular or intravascular injection (e.g., intravenous infusion) or intramuscular injection. Other routes of administration, e.g., oral (p.o.), may be used if desired and practicable for the particular emulsion to be administered.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the application.

In certain embodiments, formulations of the subject emulsions are pyrogen-free formulations which are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)).

Formulations of the subject emulsions include those suitable for oral, dietary, topical, parenteral (e.g., intravenous, intraarterial, intramuscular, subcutaneous injection), ophthalmologic (e.g., topical or intraocular), inhalation (e.g., intrabronchial, intranasal or oral inhalation, intranasal drops), rectal, and/or intravaginal administration. Other suitable methods of administration can also include rechargeable or biodegradable devices and controlled release polymeric devices. Stents, in particular, may be coated with a controlled release polymer mixed with an agent of the application. The pharmaceutical compositions of this disclosure can also be administered as part of a combinatorial therapy with other agents (either in the same formulation or in a separate formulation).

The amount of the formulation which will be therapeutically effective can be determined by standard clinical techniques. In addition, in vitro assays may optionally be employed to help identify optimal dosage ranges. The precise dose to be employed in the formulation will also depend on the route of administration. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula: Dose (mL)=[patient weight (kg)×dose level (mg/kg)/drug concentration (mg/mL)]

Therapeutics of the disclosure can be administered in a variety of unit dosage forms and their dosages will vary with the size, potency, and in vivo half-life of the particular therapeutic being administered.

For in situ applications, emulsions may be formulated to have optimal pharmacokinetic properties to enable uptake by phagocytes before clearance of the emulsion.

Doses of therapeutics of the disclosure will also vary depending on the manner of administration, the particular use of the emulsion, the overall health, condition, size, and age of the patient, and the judgment of the prescribing physician.

The formulations of the application can be distributed as articles of manufacture comprising packaging material and a pharmaceutical agent which comprises the emulsion and a pharmaceutically acceptable carrier as appropriate to the mode of administration. The pharmaceutical formulations and uses of the disclosure may be combined with any known compositions for the applications of the application.

### Diagnostic Detection Methods

Exemplary applications of the present invention include the diagnostic detection of cells, e.g., immune cells that accumulate at tissue sites as part of an inflammatory response and cells that are grafted into the body in order to treat a disease or condition, i.e., cytotherapy. Cytotherapy can generally include the administration of cells to a subject in need thereof. In some cases, the imaging method described herein is used to diagnose a disease or to determine a prognosis. Cells can be endogenous cells in the body, for example, various immune cells (T cells, B cells, macrophages, NK cells, DCs, etc.), stem cells, progenitor cells, cancer cells, as well as engineered cells, which are often used in cytotherapy in its various forms. An engineered cell can express a heterologous nucleic acid or a recombinant protein.

Non-invasive imaging of cells, e.g., immune cells in the body is useful because it can aid in the diagnosis and monitoring of disease, e.g., inflammation. In the field of cytotherapy, the ability to image the cell graft provides valuable feedback about the persistence of the graft, potential cell migration, and improves safety surveillance. Many experimental cell therapies that are in clinical trials, e.g., stem cells and immunotherapeutic cells, could benefit from the use of this technology.

### Computer Methods

Methods for quantifying labeled cells will typically be conducted with the aid of a computer, which may operate software designed for the purpose of such quantification. Such software may be a stand-alone program or it may be incorporated into other software, such as MRI image processing software. See, for example, U.S. Patent Publication No. 2007/0253910, herein incorporated by reference in its entirety.

The disclosure will be more readily understood by reference to the following examples, which are included merely for purposes of illustration, of certain aspects and embodiments of the present application, and are not intended to limit the disclosure.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### EXAMPLES

### Example 1: Cell Penetrating Peptide Functionalized Perfluorocarbon Nanoemulsions for Targeted Cell Labeling and Enhanced Fluorine-19 MRI Detection

Detailed descriptions of exemplary of the present invention of cell penetrating peptide functionalized perfluorocarbon nanoemulsions can be found in Hingorani et al., Magn Reson Med, 2020, 83:974-987, the contents are hereby incorporated in its entirety including the figures, figure legends, and supplemental information.

### Abstract

Purpose: A bottleneck in developing cell therapies for cancer is assaying cell biodistribution, persistence and survival in vivo. Ex vivo cell labeling using perfluorocarbon (PFC) nanoemulsions, paired with ¹⁹F MRI detection, is a non-invasive approach for cell product detection in vivo. Lymphocytes are small and weakly phagocytic, which can limit PFC labeling levels and MRI sensitivity. To boost labeling, we designed PFC nanoemulsion imaging probes displaying a cell-penetrating peptide, namely the transactivating transcription sequence (TAT) of the human immunodeficiency virus. We report optimized synthesis schemes for preparing TAT co-surfactant to complement the common surfactants used in PFC nanoemulsion preparations

Methods: We performed ex vivo labeling of primary human chimeric antigen receptor (CAR) T cells with nanoemulsion. Intracellular labeling was validated using electron microscopy and confocal imaging. To detect signal enhancement in vivo, labeled CAR T cells were intra-tumorally injected into mice bearing bilateral flank glioma tumors.

Results: By incorporating TAT into the nanoemulsion, a labeling efficiency of ˜10¹² fluorine atoms per CAR T cell was achieved which is a >8-fold increase compared to nanoemulsion without TAT while retaining high cell viability (˜84%). Flow cytometry phenotypic assays show that CAR T cells are unaltered after labeling with TAT nanoemulsion, and in vitro tumor cell killing assays display intact cytotoxic function. The ¹⁹F MRI signal detected from TAT-labeled CAR T cells was eight times higher than cells labeled with PFC without TAT.

Conclusion: The peptide-PFC nanoemulsion synthesis scheme presented can significantly enhance cell labeling and imaging sensitivity and is generalizable for other targeted ¹⁹F MRI imaging probes.

### Introduction

Noninvasive methods for tracking cell therapy grafts are an urgent unmet clinical need. With the development of adoptive immunotherapy against cancer, such as using chimeric antigen receptor (CAR) T cell therapy (1,2), there is a need to determine the initial biodistribution and survival of the therapeutic cells (3). Visualizing cell populations in vivo can also provide insights into off-site toxicities and help refine dosing regimens to enhance therapeutic efficacy (4,5).

The inventors have developed a novel formulation of perfluorocarbon (PFC) based emulsions that are functionalized with a peptide, or other moiety, on the emulsion droplet's surface as a component of the surfactant layer. This is the first example of peptide-PFC nanoemulsions formulated entirely from synthetic components. Prior art employs phospholipid surfactants to form nanoemulsion that mimic the membranes of live cells and impart biocompatibility. However, phospholipid-formulated emulsions are prone to instability under storage conditions due to oxidation-mediated changes to the lipid that limits shelf-life, especially if metal ions are present, and lipid oxidation by-products may lead to cytotoxicity upon cell contact. Additionally, the formulation of phospholipid-based nanoemulsions requires a time-consuming multi-step chemical process. For these reasons, peptide conjugates for use with synthetic polymeric co-surfactants are a significant over prior art. The peptide-PFC emulsion described in the present example can be used for boosting emulsion uptake into cells labeled ex vivo for use in in vivo imaging after grafting to the subject. The compositions described in the present example can also be combined and incorporate other chemical and formulation modifications. For example, the addition of fluorous metal chelates dissolved in the fluorous phase of the emulsion can impart additional functionality to peptide-PFC emulsion.

Noninvasive imaging techniques for cell detection post-transfer often employ radioisotopes (6,7), bioluminescence reporters (8), and fluorescence probes (9,10). MRI is also being adapted to visualize cells (11). MRI has no depth penetration limitations, displays anatomy with clarity, and can be used with in conjunction with imaging agents clinically (12,13).

Fluorine-19 based MRI nanoemulsion probes are an option for non-invasively imaging of cell populations (14-20). The ¹⁹F nuclei have high intrinsic sensitivity, with 89% relative sensitivity compared to ¹H. De minimis endogenous ¹⁹F in the body ensures that any MRI signals collected are from the introduced tracer probe. F-dense perfluorocarbon (PFC) molecules are often used to form nanoemulsion imaging probes that can be endocytosed by cells. As PFCs are mostly chemically inert, lipophobic, and hydrophobic, and nanoemulsions do not osmotically diffuse out of viable cells thereby ensuring lasting labeling. Detailed reviews of the biomedical applications of ¹⁹F cell detection and tracking are found elsewhere (21-24).

Engineered lymphocytes commonly used in immunotherapy (25) have an intrinsically small cytoplasmic volume and are weakly phagocytic, thereby restricting uptake of intracellular PFC label. The limits of cell detection in spin-density weighted ¹⁹F MRI is linearly proportional to the cell labeling levels. Thus, to boost cell labeling, we designed PFC nanoemulsion imaging probes displaying a cell penetrating peptide (CPP) from the transactivator of transcription (TAT) component of the human immunodeficiency virus type-I (26). TAT is an 86 amino acid protein, and residues 49-58 [Arg-Lys-Lys-Arg-Arg-Gln-Arg-Arg-Arg] are positively charged and carry a nuclear localization signal sequence facilitating endocytosis (27). We report the synthesis schemes and physical characterizations of three novel TAT co-surfactants for PFC nanoemulsion formulation. For PFC, we employ perfluoropolyether (PFPE, a perfluorinated polyethylene glycol) or perfluoro-15-crown-5-ether (PFCE); both molecules are used for ¹⁹F MRI due to unitary major fluorine peaks and high sensitivity (28,29). The efficacy of TAT co-surfactants was tested by measuring cell uptake in Jurkat T cells and in human CAR T cells. In vitro functional cell (glioma) killing assays were performed using TAT-PFC labeled CAR T cells. The intracellular localization of PFC oil droplets in labeled CAR T cells was investigated by fluorescence and electron microscopy. Additionally, we conducted proof-of-concept in vivo ¹⁹F MRI sensitivity studies in CAR T cells labeled with TAT-PFC injected into flank gliomas.

### Methods

Synthesis of PFC Nanoemulsions with CPP and Poloxamer Surfactants

To prepare CPP surfactant, 1 mmol of 1H,1H-perfluoro-1-heptanol (0.350 g, 1 mmol, mol wt=350 g/mol) or 1H,1H-perfluoro-3,6,9-trioxadecan-1-ol (0.398 g, 1 mmol, mol wt=398 g/mol, Exfluor Research, Round Rock, Tex.) was added to a 25 mL round bottom flask along with 232 mg 6-maleimidohexanoic acid (232 mg, 1.1 equiv, 1.1 mmol, mol wt=211.32 g/mol, TCI America, Portland, Oreg.). Anhydrous dichloromethane (5 mL) was added, and the flask was maintained under a constant stream of N₂ gas while stirring. Once the reactants dissolved, 572.4 mg benzotriazol-1-yl-oxytripyrrolidinophosphonium hexafluorophosphate (PyBOP, 572.4 mg, 1.1 equiv, 1.1 mmol, mol wt=520.39 g/mol, Bachem, Torrance, Calif.) was added in one portion. After 2 min for the coupling reagent to dissolve, 350 μL of diisopropylethylamine (DIEA, 2 equiv, 2 mmol, Sigma Aldrich, St Louis, Mo.) was added to start the reaction. The flask was left under a slow N₂ stream with constant stirring at room temperature for 16 h. The reaction completion was monitored by thin layer chromatography (TLC, R_{f}=0.4, 3:7 EtOAc:hexanes). Purification and solvent removal were accomplished using a Combiflash Rf Lumen (Teledyne Isco, Lincoln, Nebr.) silica gel column (12 g, silica Redisep column) using a hexane and ethyl acetate gradient with 1:0 hexane:EtOAc for 3 min, followed by an increase in polarity to 1:1 hexane:EtOAc from 3 min to 14 min, followed by a 0:1 hexane:EtOAc wash for 1 min. An evaporative light scattering (ELS) detector was used for monitoring product peaks, at 250 nm and 280 nm wavelengths, which elute at retention times (t_{R})=9 min (1) and t_{R}=10.5 min (2), respectively. The collected fractions were concentrated with a rotary evaporator and dried on high vacuum overnight. The products are a clear oil with mol wt=591.28 g/mol (1) (yield=325 mg) and mol wt=543.28 g/mol (2) (yield=380.1 mg).

Cys-TAT.9TFA (30 mg, 0.016 mmol, 1 equiv, mol wt=2688.16 g/mol, Biomatik, Wilmington, DL) was dissolved in 464 μL of 0.05% TFA-water. A solution of 1 or 2 (0.014 mmol) in trifluoroethanol (556 μL) was then added to the solution of Cys-TAT, followed by the addition of 116 μL of 1 M 3-(N-morpholino)propanesulfonic acid (MOPS) buffer at pH=7.4. Reaction completion was assessed by liquid chromatography mass spectroscopy (LC-MS, Model 1100 with LC/MSD Trap, Agilent, Santa Clara, Calif.) using a 95:5 gradient of water+0.05% TFA:acetonitrile+0.05% TFA for 5 min, then 95:5 to 10:90 in 20 min, followed by 10:90 to 0:100 in 10 min, t_{R}=18.5 min (1) and t_{R}=18.6 min (2)] and stopped after 30 min by addition of 100 μL of glacial acetic acid. Following filtration (0.22 μm nylon filter), the crude mixture was purified by semi-prep high pressure liquid chromatography [HPLC, gradients used: 90:10 descending to 10:90 water+0.05% TFA, acetonitrile+0.05% TFA in 20 min, t_{R}=12.5 min, m/z=1127.4, mol wt=2254.28 g/mol (1a, TATP) and t_{R}=13.7, m/z=1103.5, mol wt=2206.28 g/mol (2a, TATA)].

To prepare nanoemulsions, a 5% w/w ratio of total surfactant to PFC was used. For 4 mL of nanoemulsion product, 40 mg of polyethylene-polypropylene (F68) in 400 μL of water was added to a glass vial containing 465 μL PFCE (Exfluor). To this solution, 4 mg (1.21 μmol) of TATP (1a) or TATA (2a) (1.23 μmol) was added followed by 3.135 mL of purified water. The solution was ultrasonicated (30% power, 1 min, Omni Ruptor 250W, Kennesaw, GA) and then passed through a microfluidizer (LV1, Microfluidics, Westwood, Mass.) at 10,000 psi pressure four times. The TATA- and TATP-F68-PFC (3) nanoemulsions were sterile filtered using a 0.22 μm syringe filter (Acrodisc PF, Pall, Port Washington, N.Y.) and bottled in autoclaved glass vials. The capped vials were stored at 4° C. until use.

Synthesis of PFC Nanoemulsions with CPP-Phospholipid Surfactants

For CPP-phospholipid surfactant, 1,2-distearoyl-sn-glycero-3-phosphoethanolamine-N-[maleimide(polyethyleneglycol)-2000] (DSPE-PEG2000-maleimide, 1.8 equiv., 0.00217 mmol, 6.5 mg, Avanti Polar Lipids, Alabaster, Ala.) was suspended in HEPES buffer (0.05 M, 500 μL, pH=7.5) by sonication. A fresh solution of Cys-TAT (1 equiv, 0.0012 mmol, 2.0 mg) in HEPES buffer (0.05 M, 300 μL, pH=7.5) was added in one portion, and the mixture was agitated on a shaker at 37° C. for 6 hours. 2-Mercaptoethanol (0.8 μL, 10 equiv, 0.012 mmol, Sigma Aldrich) was added to react with any remaining maleimide groups, and the solution was agitated further for 30 min.

The conjugate was de-salted and purified in deionized water using a dialysis cassette (Slide-A-Lyzer #2K MWCO, cassette size=3 mL, Thermo Fisher Scientific, Waltham, Mass.) at room temperature. Water was replaced at 2, 4 and 22 hours (volume 300.1 compared to cassette size). The sample was recovered from cassette and analyzed by matrix assisted laser desorption/ionization (MALDI) mass spectrometry (Biflex IV MALDI-TOFMS, Bruker, Billerica, Mass.) and identified as a mixture of the desired product, DSPE-PEG(2000)-Cys-TAT (mol wt=4660 g/mol) 4 and DSPE-PEG(2000)-mercaptoethanol (mol wt=3020 g/mol). The solution was lyophilized to a dry powder to give a near-quantitative yield of the desired product, 4.

The phospholipid-PEG-TAT conjugate was incorporated into egg yolk phospholipid (EYP) by the two methods described below. For both methods, compound 1 (2.8 mg, 0.6 μmol) and EYP (304 mg, 0.4 mmol, Sigma Aldrich) were mixed resulting in a TAT to lipid surfactant ratio of 0.15 mol %. Thereafter, PFPE oil (1.18 g, 0.87 mmol, mol wt=1300-1400 g/mol, Exfluor) was added to obtain a 26% w/w ratio of phospholipid to PFPE. Sterile water was added to obtain a 120-150 mg/mL concentration of PFPE. The nanoemulsions were then sterile-filtered through (0.2 μm, Pall) into glass vials, capped, and stored at 4° C. until use. Following formulation and filtration, each nanoemulsion was characterized by dynamic light scattering (DLS) particle analysis and ¹⁹F NMR (see Supporting Methods).

### Method 1: Direct Insertion of Peptide Conjugate

Compound 4 was added to a solution of EYP in chloroform (5 mL), vortexed on medium for 1 min, and the resulting solution evaporated with a stream of nitrogen while manually rotating the vessel. The vial was then placed under high vacuum overnight to give a dry lipid film. Sterile water was added to hydrate the lipid film for 5 min followed by vortexing on medium for 2 min and then ultrasonication (30% power, 4 min). PFPE was added to the vial in one portion, vortexed briefly, and then ultrasonicated (30% power, 2 min). The crude emulsion (5) was passed four times through a microfluidizer at 20,000 psi with the reaction chamber cooled on ice.

### Method 2: Post-Insertion of Peptide Conjugate

A suspension of EYP in sterile water was formed by ultrasonication (30% power, 4 min), and PFPE oil was added to the vial in one portion, vortexed briefly, and then ultrasonicated (30% power, 2 min). The crude emulsion was passed four times through a microfluidizer as in method 1. To incorporate TAT, solutions of 1 based on mol % of total EYP surfactant were prepared in sterile water. The solution of 1 is added to the preformed nanoemulsion and agitated on a bioshaker at 37° C. for 5 h to obtain (5) nanoemulsion.

### T Cell Preparation

The Jurkat T cell line was obtained commercially (#TIB-152, ATCC, Manassas, Va.) for initial nanoemulsion cell labeling characterizations. Jurkat cells were grown in RPMI-1640 media (Gibco, Waltham, Mass.) plus 10% fetal bovine serum (FBS), 10 mM HEPES buffer (4-(2-hydroxyethyl)-1-piperazineethanesulfonic acid), 1 mM sodium pyruvate and 1.5 mg/mL sodium bicarbonate.

Primary human T cells were obtained from blood samples sourced from the San Diego Blood Bank and enriched for T cells by Ficoll (Histopaque 1077, Sigma Aldrich) gradient density centrifugation and magnetic assisted cell sorting (Dynabeads, Thermo Fisher). T-cells were then activated with human T-activator CD3/CD28 Dynabeads and allowed to expand for two days in RPMI-1640 supplemented with 10% FBS and 100 units/mL of recombinant human interleukin 2 (IL-2, Peprotech, Rocky Hill, N.J.). For transduction, we employed a vector specific to epidermal growth factor receptor variant III (EGFR-vIII) as described by Johnson et al. (30). A detailed CAR virus production and human T cell transduction is available elsewhere (31). CAR receptor expression was confirmed by flow cytometry. We used T cell populations with >70% CAR+ expression.

### Glioma Cell Line

A human glioblastoma multiform (U87-EGFRvIII-Luc) cell line overexpressing EGFR-vIII (32) and the luciferase reporter gene (Luc) were used. Cells were incubated (37° C., 5% CO₂) and cultured in T-75 flasks (Thermo Fisher) in RPMI-1640 medium supplemented with 10% FBS.

### In Vitro T Cell Labeling

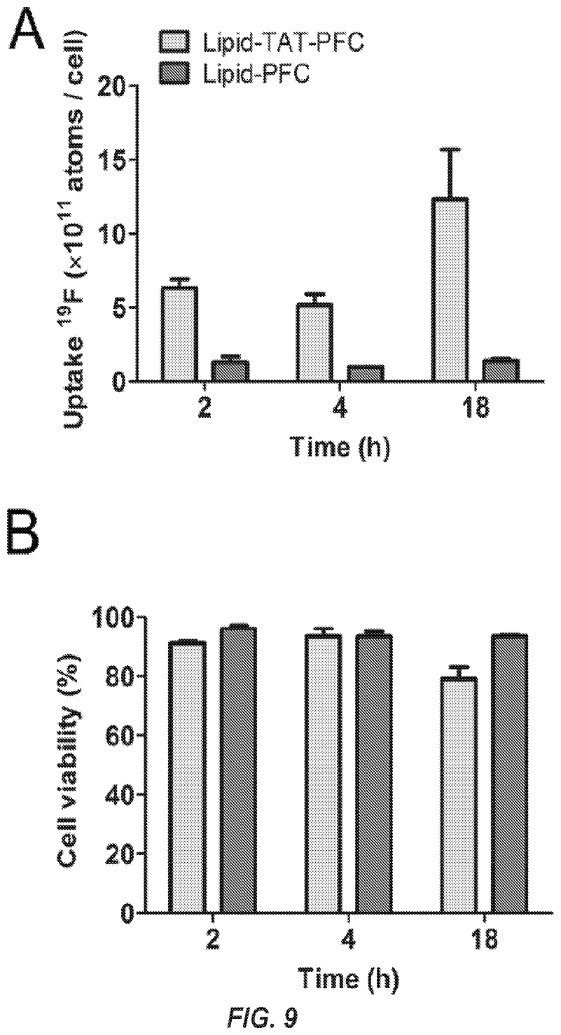

For initial uptake experiments, 1 million Jurkat or CAR T cells were plated in 1 mL full media in 24 well plates (n=3 wells per condition). PFC nanoemulsion was added to each well and incubated overnight (16 h) at 37° C. and 5% CO₂. The cells were then washed three times with phosphate buffered saline (PBS) to remove free nanoemulsion. Cells were counted and viability was assessed by Trypan blue staining. Thereafter, the cells were spun down, resuspended in 150 μL lysis buffer (1% Triton X in PBS) and transferred to 5 mm NMR tubes. 50 μL 0.1% TFA was added to each NMR tube, and spectra were acquired (see Supporting Methods). Total fluorine atom count was divided by the sample cell number to yield the average number of fluorine atoms per cell (FIG. 9A).

For in vivo experiments, CAR T cells were plated at a density of 10 million cells in 5 mL of media per well of a 6-well plate and incubated overnight with 15 mg/mL of nanoemulsion. Cells were washed as described above and an aliquot of 1 million cells was set aside to measure cell uptake by ¹⁹F NMR.

### Synthesis of Cyanine-5 (Cy5) Fluorescence Nanoemulsions

TAT-PFC nanoemulsions were prepared with Cy5 dye attached (FIG. 10). To 0.5 mmol of 1H,1H-Perfluoro-1-heptanol (mol wt=350.08 g/mol) or 1H,1H-Perfluoro-3,6,9-trioxadecan-1-ol (mol wt=398.08 g/mol), we added 0.55 mmol of 6-(Boc-amino)caproic acid (mol wt=231.29 g/mol, Sigma Aldrich); this mixture was dissolved in a minimum amount of dry dichloromethane (DCM), and the reaction mix was stirred under N₂ gas. 0.55 mmol pyBOB (benzotriazol-1-yl-oxytripyrrolidinophosphonium hexafluorophosphate) was added, followed by 0.74 mmol of diisopropylethylamine (DIEA), and mixture was stirred under inert gas overnight at room temperature. Reaction completion was monitored by TLC. The solvent was removed with a rotary evaporator, and sample was dissolved in minimum amount of DCM. Wet crude sample was loaded on a 4 gm silica gel Redisep column for purification using the Combiflash Rf Lumen. We eluted with 100% hexane for 2 min, then 70%:30% EtOAc:hexane over 10 min. The desired product 6 or 7 was eluted between 30-40% EtOAc and t_{R}=6-7.5 min, as monitored by ELS detector.

Boc protecting group in compound 6 or 7 was removed by adding 1 mL of TFA and 3 mL of DCM while stirring at room temperature for 1 h. The TFA was removed by forming an azeotrope with toluene, and the sample was dried under a rotary evaporator followed by high vacuum to extract all solvents. LC-MS used 10:90 to 90:10 Acetonitrile+0.05% TFA:1120 in 20 min. The purified compound eluted at t_{R}=15.3 min, m/z=515.3 (6a) and t_{R}=16.6 min, m/z=564.0 (7a) using the 215 nm detector.

A 25 mM stock solution of 6a or 7a was prepared by dissolving weighed oil in calculated amount of trifluoroethanol. We used 8 μL of 6 mM Cy5-N-hydroxysuccinimide (Cy5-NHS, 48 nmol, GE Healthcare, Chicago, Ill.) and an excess of 6a or 7a (approximately 20 equiv, 960 nmol or 38.5 μl of 25 mM stock prepared above) was added. The molar equivalent amount of N-methyl morpholine was added, prepared as a 50 mM solution in DMSO. The reaction was stirred at room temperature overnight. Thereafter, 2 μl of acetic acid was added, and the reaction mix was purified by HPLC (gradient 10:90 to 90:10 water+0.05% TFA:Acetonitrile+0.05% in 20 min and retain at 90:10 for an additional 10 min on a Phenomenex Luna 5 μm C18(2) 100 Å, 250×10 mm column). The desired product was eluted at t_{R}=21.6 min, m/z=1150.3 (8) and t_{R}=20.3 min, m/z=1102.3 min (9), as monitored by UV absorbance at 650 nm.

To prepare nanoemulsions, we used the procedure as described above. Prior to sonication, 0.3 μM of 8 is added to cocktail where TATP is used as the chosen anchor. Similarly, 0.3 μM of 9 is added to cocktail using TATA. Following sonication and microfluidization a faint blue nanoemulsion is obtained.

### In Vivo MRI

All animal protocols were approved by the University of California, San Diego, Institutional Animal Care and Use Committee (IACUC). Bilateral subcutaneous flank tumors were implanted in N=4 female NOD/SCID 4-6 week old mice (Jackson Laboratories, Bar Harbor, Me.). Tumor inoculant consisted of 5×10⁶ U87-EGFRvIII-Luc tumor cells in 100 μL matrigel (Corning, Tewksbury, Mass.) in PBS (1:1). Five days later, mice received intra-tumoral injection of 1×10⁷ labeled CAR T cells labeled with either TATP-F68-PFC or F68-PFC nanoemulsion. Two hours after intratumoral injection, mice were anesthetized with 1-2% isoflurane in O₂ and positioned an 11.7 T Bruker BioSpec preclinical scanner with a dual-tuned ¹H/¹⁹F birdcage volume coil. Animal temperature was regulated, and respiration was monitored during scans. A reference capillary with dilute PFC nanoemulsion was positioned in the image field of view (FOV). ¹H anatomical images were acquired using the RARE (rapid acquisition with relaxation enhancement) sequence with TR/TE=2000/13 ms, RARE factor 4, matrix 256×184, FOV 38×30 mm², slice thickness 1 mm, 18 slices, and 4 averages. The ¹⁹F images were also acquired using a RARE sequence with parameters TR/TE=1500/4.7 ms, RARE factor 8, matrix 64×46, FOV 38×30 mm², slice thickness 1 mm, 18 slices, and 400 averages. The total number of fluorine atoms per voxel in tumor regions were estimated directly from the vivo ¹⁹F image hot-spots using the software program Voxel Tracker (Celsense, Pittsburgh, Pa.), which also employs image measurements of the external ¹⁹F reference capillary signal and noise as inputs, and yields a statistical uncertainty of ¹⁹F-count; additional details are published elsewhere (33). For display, ¹⁹F images were manually thresholded to remove background noise, and ¹H/¹⁹F renderings were performed in ImageJ by overlaying ¹H (grayscale) and ¹⁹F (hot-iron scale) slices. Regions of interest (ROI) were segmented around relevant ¹⁹F signals (right tumor, left tumor and noise), and ROI voxel intensities were displayed as histograms.

### Statistical Analyses

Measurements are presented as mean±standard deviation. We performed unpaired T-tests with unequal variances to compare in vivo groups. Two tailed P-values<0.05 were considered statistically significant.

### Supporting Methods

### Synthesis of F68-TAT

3.78 g of polyethylene-polypropylene (F68, 1 equiv, 0.0453 mmol, mol wt=8350 g/mol, Spectrum Chemicals, Gardena, Calif.), purchased as a solid, was further dried under high vacuum for 1 hour prior to use. To this dried white powder, 25 mL of anhydrous dichloromethane was added and stirred until dissolution, yielding a clear solution. Reaction was maintained under dry conditions using a steady stream of N₂ gas. 172 mg of 6-maleimidohexanoic acid (1.8 equiv, 0.815 mmol) was added in one portion yielding a pale yellow solution. 0.906 mL of N, N′-Dicyclohexylcarbodiimide was added dropwise; solution cloudiness and formation of precipitate was observed almost immediately. The reaction was stirred overnight at room temperature under inert gas. Reaction was monitored by thin layer chromatography (TLC) with product retention factor (R_{f})=0.1 in 20:80:0.5 mix of MeOH:CHCl₃:AcOH. Reaction byproduct was removed by filtration. To the filtrate, an excess of hexanes was added causing the product to precipitate, which is then collected by filtration. Product is a pale pink solid, 10 (yield=2.183 g, mol wt=8493 g/mol). To 2.5 mg of 10 (1 equiv, 0.00029 mmol) prepared as a 5 mg/mL solution in distilled water, 0.3 mg of Cys-TAT (0.68 equiv, 0.00020 mmol) prepared as a 2 mg/mL solution in HEPES buffer was added and stirred overnight. Thereafter, 10 equiv of cysteine was added to cap any remaining maleimide groups. The contents of the reaction vessel were dialyzed (Slide-A-Lyzer Dialysis Cassettes, 3.5K MWCO, 3 mL, Thermo Fisher Scientific, Rockford, Ill.) to remove residual Cys-Tat (mol wt=1661.99 g/mol) and cysteine (mol wt=121 g/mol).

### Nanoemulsion Size and Homogeneity

Dynamic light scattering instrumentation (Zetasizer ZS, Malvern Panalytical, Malvern, UK) was used to determine the particle size, polydispersity index (PDI) and zeta potential for the samples. Nanoemulsions were diluted to 0.5% v/v in water and transferred to low volume (1.5 mL) disposable cuvettes. Measurements were performed in triplicate samples for each nanoemulsion.

### ¹⁹F NMR Measurement of Nanoemulsion Fluorine Concentration

The ¹⁹F NMR spectral data were acquired using a 400 MHz Bruker NanoBay Spectrometer (Bruker BioSpin, Billerica, Mass.) with a single 17 μs pulse, 32,000 free induction decay points, 100 ppm spectral width, 32 averages and 15 s repetition time. NMR samples were prepared by adding 0.1% (w/v) sodium TFA in D₂O to nanoemulsion (10% v/v). The concentration of PFC in nanoemulsion (C_{F}) was calculated from the integrals of the TFA signal (normalized to 1) and the major PFC peak (I_{PFC}) using the relation C_{F}=a I_{PFC} C_{TFA} V_{TFA} (b V_{PFC})⁻¹, where C_{TFA} is the concentration of TFA in mg/mL, V_{TFA} and V_{PFC} respective volumes of nanoemulsion and 0.1% TFA solution in mL in the NMR sample, and a and b are constants representing the ¹⁹F mass fraction of TFA (0.42) and PFC (e.g., 0.655 for PFCE and 0.58 for PFPE), respectively.

### Fluorescence Microscopy

Aliquots of Chimeric Antigen Receptor (CAR) T cells (1×10⁶ cells, N=3) were labeled with Cy5-TATP-F68-PFC nanoemulsion or mock-labeled for a 24 h period and then fixed with 4% paraformaldehyde for 20 min. Cells were stained with CD3-Alexa488 (1:200 dilution, Biolegend, San Diego, Calif.) and Hoechst dye nuclear stain (#33342, 1:500 dilution, Thermo Fisher). Cells were then mounted in Aqua-Mount media (Lerner Laboratories, Cheshire, Wash.), and slides were imaged using a confocal microscope (Model A1, Nikon Instruments, Melville, N.Y.) with a 40× water immersion objective and 405 nm, 488 nm and 640 nm lasers for excitation.

### Electron Microscopy

To confirm intracellular PFC localization, we examined CAR T cells labeled with TATP-F68-PFC or TATA-F68-PFC alongside unlabeled CAR T cells using electron microscopy. Cells were labeled as above. CAR T cell pellets were fixed in PBS containing 2% glutaraldehyde in 0.1 M sodium cacodylate (SC) buffer at room temperature for 30 min and stored overnight at 4° C. The cells were washed five times in 0.1 M SC buffer on ice and treated with 1% OsO₄ in 0.1 M SC buffer for 1 hour. All samples were washed in deionized water and treated with 2% uranyl acetate for 1 hour on ice. Pellets were dehydrated in ethanol and then anhydrous acetone. The cells were embedded in a solution containing a 1:1 mixture of acetone and Durcupan resin (Sigma Aldrich) for 2 h on a tube rotator and then in 100% Durcupan overnight. The next day, cell pellets were embedded in Durcupan resin and polymerized over 36 h at 60° C. Ultra-thin (60 nm) sections were cut using a diamond knife and collected on Cu mesh grids. The samples were stained with 1% aqueous uranyl acetate and Reynolds lead citrate. Sections were imaged using a Tecnai Spirit electron microscope (FEI, Hillsboro, Oreg.) at 80 kV.

### Flow Cytometry

Potential impact of TATP-F68-PFC on CAR T cell phenotype and viability was evaluated by measuring surface expression levels of CD3, CD4, CD8, and 7-AAD (viability marker) by flow cytometry (LSR Fortessa, BD Biosciences, San Jose, Calif.). We used Alexa488 anti-human CD3 clone HIT3a, Phycoerythrin-cyanine 5 (PE-Cy5) anti-human CD4 clone OKT4, Fluorescein isothiocyanate (FITC) anti-human CD8 clone SK1, and 7-Aminoactinomycin D (7-AAD) viability marker (all purchased from Biolegend, San Diego, Calif.). In these assays, we evaluated CAR T cells without PFC label as controls. Flow cytometry data processing used FlowJo software (FlowJo, Ashland, Oreg.).

### Tumor Cell Killing Assay

U87-EGFRvIII-Luc glioma cells were plated at a density of 30,000 cells per well in clear bottom 96-well plates (Corning, Inc., Corning, N.Y.) and were allowed to adhere overnight (60 wells total for two time points). Wells (n=6, per condition) received: (i) 5:1 ratio of CAR T cells to cancer cells, (ii) 5:1 ratio of TATP-F68-PFC-labeled CAR T cells to cancer cells, (iii) 5:1 ratio of untransduced T cells to cancer cells, (iv) 5:1 ratio of TATP-F68-PFC-labeled untransduced T cells to cancer cells or (v) untreated cancer cells for baseline signal. At 12 and 24 hours post T cell treatment, D-luciferin (300 μg/mL, Biosynth International, Itasca, IL) was added to wells, and bioluminescence signals were immediately measured with a plate reader (Infinite M200PRO, Tecan, Morrisville, N.C.). The relative U87-EGFRvIII-Luc cell killing efficacy was obtained from the mean photon count for groups (i-iv); the numerical differences between treated versus untreated (v) means are displayed as percentages.

### Ex Vivo Microimaging of Tumors

Two days after intratumoral injection, animals were sacrificed by CO₂ inhalation and tumors were excised with the surrounding skin tissue. Tumors were fixed in 4% paraformaldehyde overnight, rinsed in PBS, and transferred to a 10 mm NMR tube (Wilman Labglass, Vineland, N.J.) containing a 2% agarose solution (Fisher Scientific, Hampton, N.H.). Images of the tumors were acquired on a Bruker 400 MHz NanoBay NMR spectrometer equipped with microimaging accessories and a 10 mm ¹⁹F/¹H microimaging coil. Proton anatomical images were acquired with a three-dimensional (3D) spin-echo sequence using TR/TE=800/25 ms, matrix 256×128×128, FOV 9.5×9.5×12 mm³ and 1 average. Fluorine images were acquired with a 3D RARE sequence using TR/TE=2000/9.7 ms, RARE factor 4, matrix 128×64×64, FOV 9.5×9.5×12 mm³ and 35 averages. The ¹H/¹⁹F image overlays were performed in ImageJ.

### Results

### Synthesis and Characterization of TAT PFC Nanoemulsions

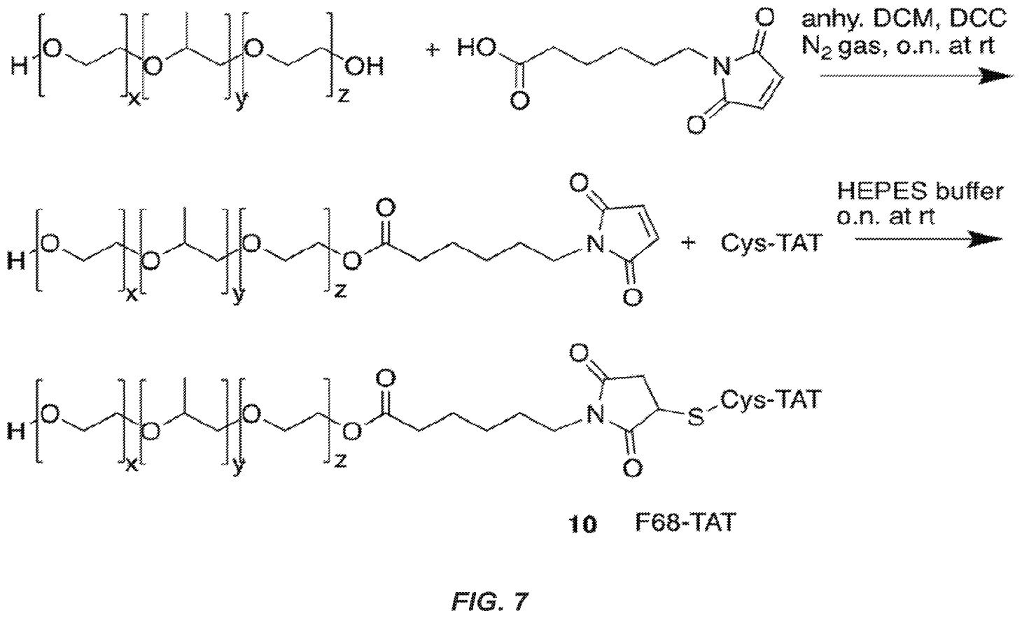

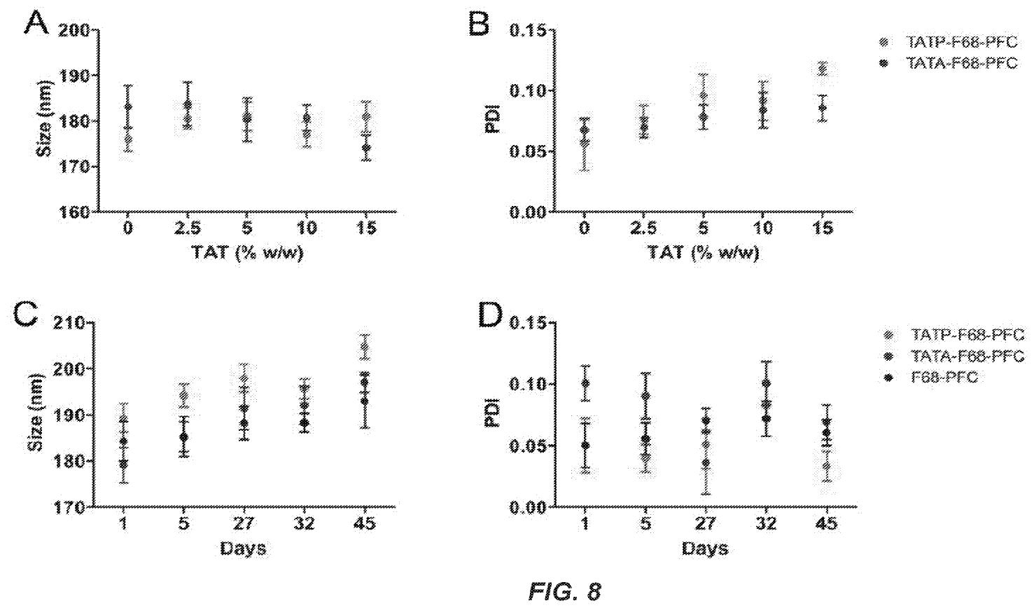

To increase the cellular uptake of PFC nanoemulsion, we chemically modified and attached the TAT peptide to the surfactant to display the hydrophilic and positively charged cell penetrating moiety on the nanoemulsion surface. We tested two general methods (FIG. 1A, FIG. 1B) for peptide incorporation into PFC nanoemulsions; these were formed with either Pluronic F68 or phospholipid. For the poloxamer surfactant, we first conjugated TAT with a terminal cysteine (TAT-cys) directly to F68 functionalized with a maleimide group (F68-TAT, FIG. 7) (34). Alternative PFC nanoemulsions formed with F68-TAT incorporated at ≥2% w/w of the F68 surfactant lacked long term stability and nanoemulsions that had <1% F68-TAT resulted in negligible cell uptake (data not shown). A successful approach involved conjugation of Cys-TAT to one of two small fluorous molecule anchors via a short hydrocarbon linker (FIG. 1A) bearing a maleimide group that was synthesized from the corresponding alcohols and PyBOP as a conjugation agent. The two anchors consisted of either a perfluoroheptyl (TATA) or a short perfluoroPEG group (TATP), designated TATA-F68-PFC and TATP-F68-PFC, respectively, with variable percentages by weight (% w/w). Following emulsification of PFC and surfactants, both nanoemulsion formulations yielded an average size particle of 180 nm (FIG. 8A, FIG. 8B) with a polydispersity index (PDI) of 0.0795-0.095, measured by light scattering methods (see Supporting Methods), and particle size slightly increased by an average of 9% by day 45 post synthesis, but stabilized, and did not separate into fluorous and aqueous phases over three months (FIG. 8C and FIG. 8D).

Cell Labeling with TAT Poloxamer Nanoemulsion

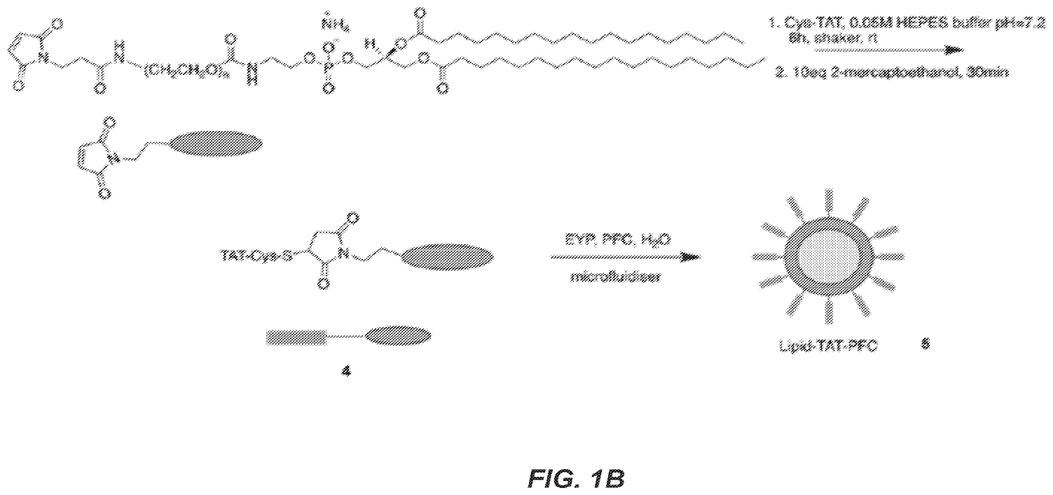

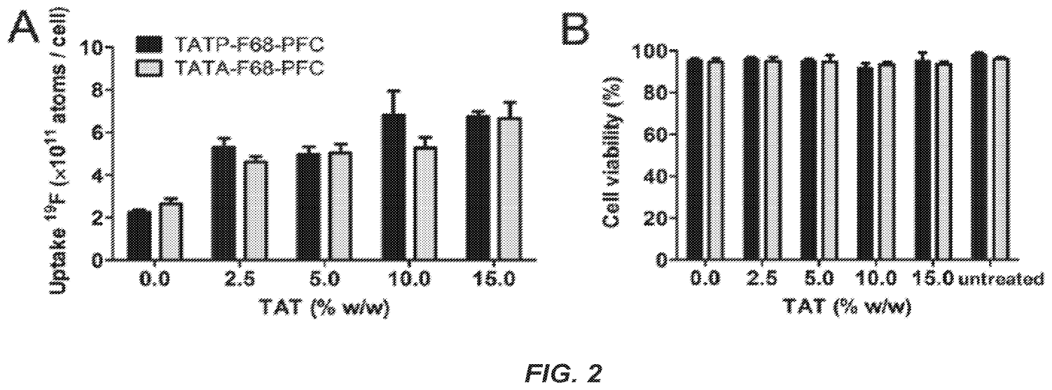

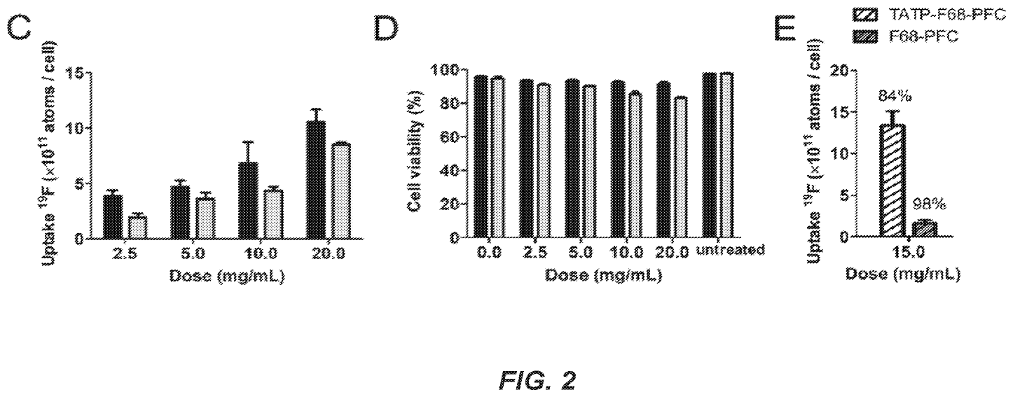

Incorporation of the TAT anchored co-surfactants into F68 formulated PFC nanoemulsions resulted in significantly enhanced uptake in Jurkat cells following an 18 hour incubation (FIG. 2A-FIG. 2E). At low CPP stoichiometry in nanoemulsion (2.5% w/w TAT), uptake was 5.33 (±0.71)×10^{11 19}F/cell for TATP-F68-PFC and 4.67 (±0.59)×10^{11 19}F/cell for TATA-F68-PFC (dose 10 mg/ml in media, FIG. 2A), measured using ¹⁹F NMR of cell pellets. For higher nanoemulsion CPP content (10% w/w TATP), cell uptake was increased to 6.82 (±1.92)×10¹¹ F/cell compared to 2.25 (±0.15)×10^{11 19}F/cell for control F68-PFC nanoemulsion; similarly, TATA-F68-PFC nanoemulsion yielded uptake values of 5.26 (±0.86)×10^{11 19}F/cell (FIG. 2A). Addition of either TATP or TATA did not impair cell viability (FIG. 2B) as measured by permeability to Trypan blue. Incubation of Jurkat cells with TATP-F68-PFC or TATA-F68-PFC with increasing concentrations of nanoemulsion in culture displays a canonical sigmoidal uptake pattern (p<0.01) (FIG. 2C). Activated CAR T cells labeled with TATP-F68-PFC at 15 mg/ml exhibit an average 8.2-fold uptake improvement compared to control F68-PFC-labeled cells (FIG. 2E). TATA-harboring nanoemulsions exhibited mild toxicity to cells with a decrease in cell viability to 85.3% at 10 mg/mL and 83% at 20 mg/mL compared to 97.7% for untreated cells (FIG. 2D). At the same doses, TATP containing nanoemulsions remained non-toxic to the cells with viability of 92% for 10 mg/mL dose and 91.7% for 20 mg/mL dose (FIG. 2D).

Cell Labeling with Phospholipid TAT-PFC Nanoemulsion

Alternative PFC nanoemulsions were also formed with EYP surfactant, where cys-TAT was conjugated to commercially available 1,2-Distearoyl-sn-glycero-3-phosphoethanolamine-peg2000-maleimide (35) (FIG. 1B), and the product 4 was confirmed by Matrix Assisted Laser Desorption/Ionization mass spectrometry. Conjugate 4 was inserted into nanoemulsion either into the crude mix prior to emulsification (“direct insertion”) or after emulsification (“post-insertion”). Formed nanoemulsions containing the adduct up to 0.15 mol % (of EYP) averaged 160 nm in size with a PDI˜0.2 immediately after emulsification; particle size did not change significantly over >10 weeks. No differences in size or zeta potential were observed for lipid-based nanoemulsions prepared with or without anchored TAT.

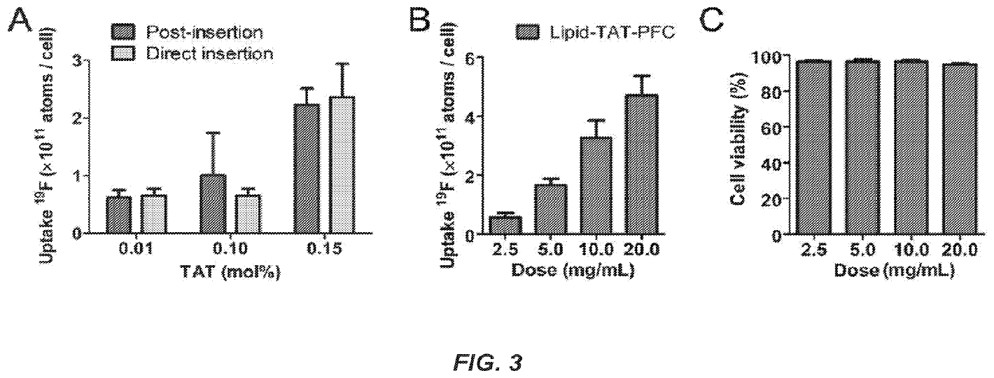

Cell labeling using TAT-modified pegylated phospholipid incorporated into EYP-surfactant nanoemulsions also resulted in greater uptake in Jurkat cells (FIG. 3A). Uptake was comparable for nanoemulsions irrespective of the method of incorporation (i.e., direct insertion or post-insertion, FIG. 3A). The ‘optimal’ incubation time was approximately 18 hours for 0.15 mol % phospholipid-TAT-PFC resulting in 1.23 (±0.85)×10^{12 19}F/cell. Shorter incubation times of 2 and 4 hours displayed lower uptake of 6.32 (±0.86)×10^{11 19}F/cell and 5.15 (±1.06)×10^{11 19}F/cell, respectively (FIG. 9A). Following 18 hours of incubation, a modest reduction in cell viability was noticed and measured to be 79 (±5.65) % for phospholipid-TAT-PFC compared to 93.5 (±0.71) % for control nanoemulsion (FIG. 9B). To test dose-dependent uptake, phospholipid-TAT-PFC (0.15 mol % of EYP) nanoemulsion was incubated with Jurkat cells for 18 hours at varying doses of 2.5, 5, 10 and 20 mg/mL. Uptake values followed a sigmoidal increase where cell uptake saturated at a dose of 10-15 mg/mL (FIG. 3B), with minimal loss in cell viability even at high doses (FIG. 3C).

### Intracellular Localization of TAT-PFC Nanoemulsion in CAR T Cells

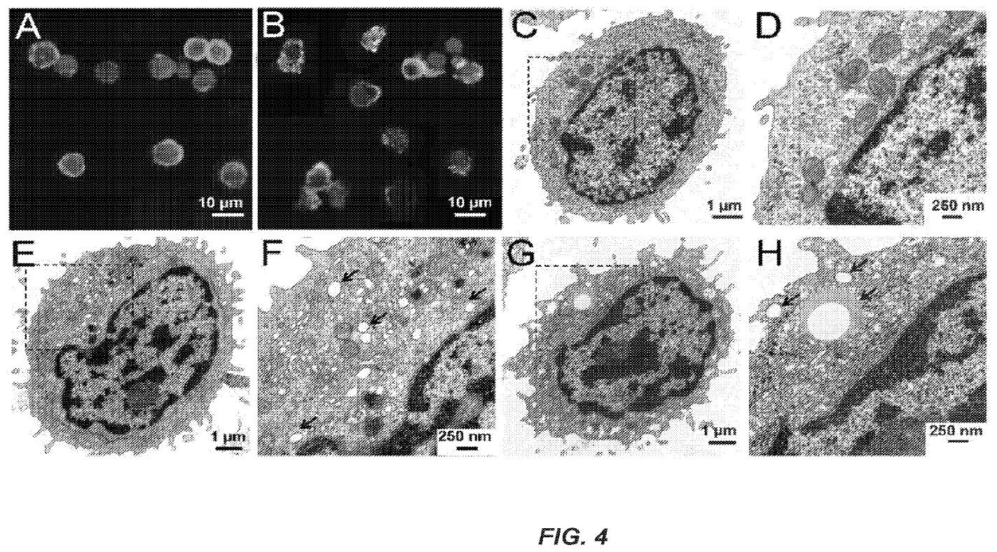

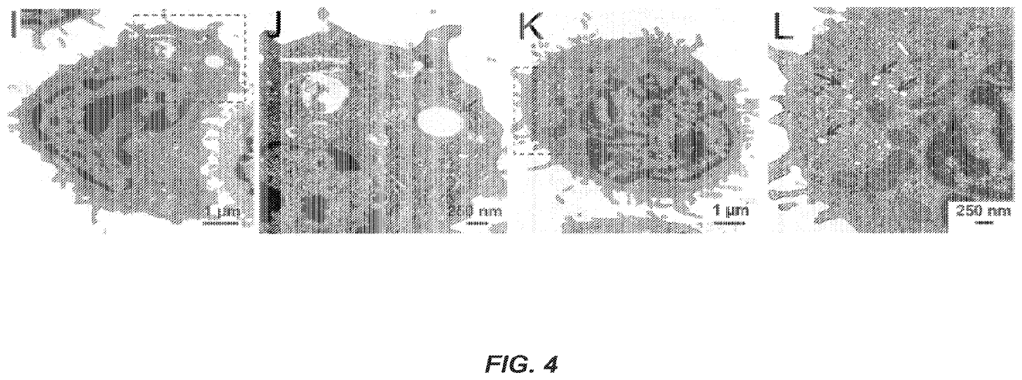

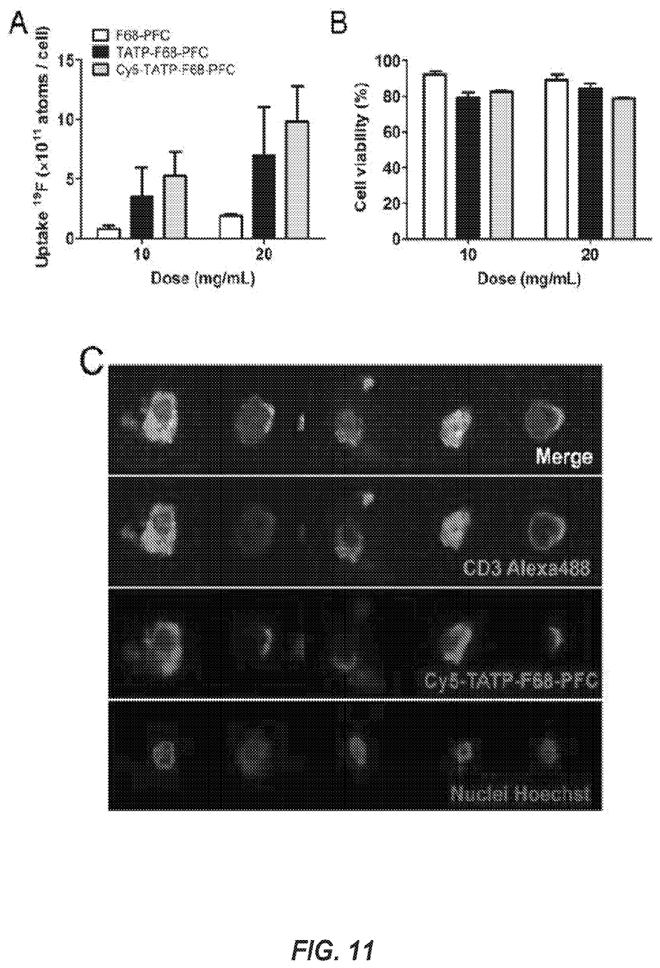

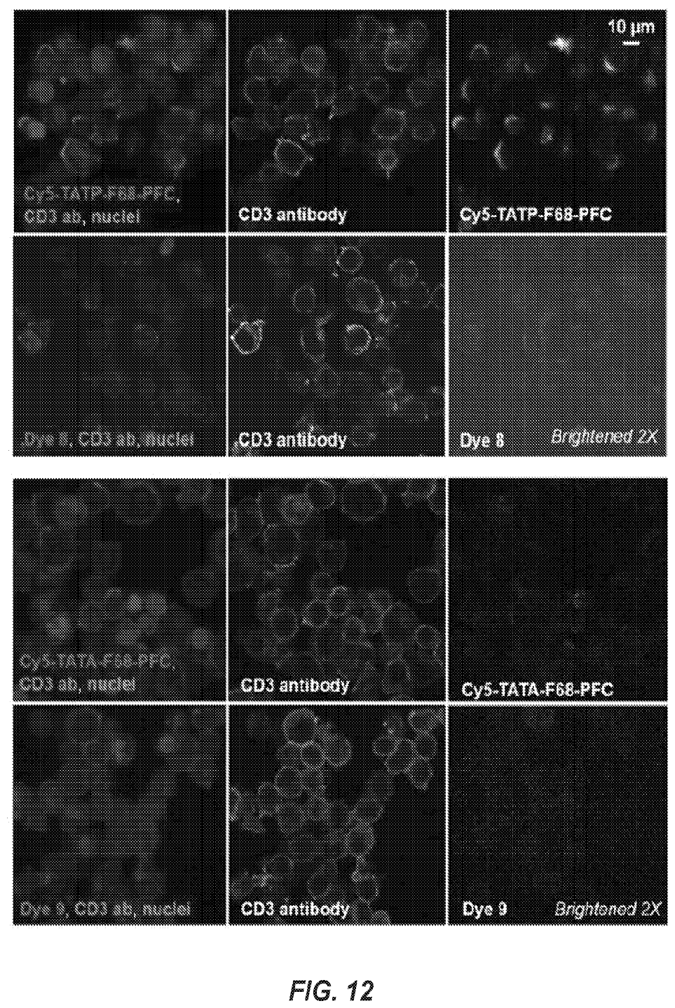

We next investigated cellular localization of TATP- and TATA-F68-PFC nanoemulsions in primary T cells using high-resolution fluorescence and electron microscopy. We synthesized fluorescently labeled co-surfactants 8 and 9 consisting of Cy5 dye attached to the respective fluorous anchors (FIG. 10) for incorporation into TATP-F68-PFC and TATA-F68-PFC nanoemulsions. Confocal microscopy of CAR T cells incubated with fluorescent nanoemulsions reveal intracellular and partial cell membrane localization of nanoemulsion (FIG. 4B) compared to untreated control cells (FIG. 4A); cells were co-stained with Hoechst nuclei stain and anti-CD3 fluorescent antibody for cell surface. As a further control, we tested whether the introduction of a surface dye on nanoemulsions enhanced cell uptake or caused overt cytotoxicity (FIG. 11A-FIG. 11C). The uptake of nanoemulsions formulated with dye co-surfactant are comparable to TATP-F68-PFC (p>0.05, FIG. 11A), retains cell viability (FIG. 11B), and compounds 8 and 9 do not appear to enhance internalization into live cells (FIG. 12).

To substantiate the intracellular localization of TAT nanoemulsion in CAR T cells, electron microscopy was performed (see Supporting Methods). The TATP-F68-PFC nanoemulsion droplets are present intracellularly and appear as clusters of small (˜100-200 nm) punctate regions of hyperintensity in micrographs (FIG. 4E, FIG. 4F), along with a few larger PFC deposits (˜1 μm) (FIG. 4G, FIG. 4H), presumably coalesced droplets, consistent with previous studies (29,36). Untreated control cells did not contain these hyperintense features in micrographs (FIG. 4C, FIG. 4D). Similar findings were observed for CAR T cells labeled with TATA-F68-PFC nanoemulsion (FIG. 4I-FIG. 4L). Normal cellular, mitochondrial and Golgi body morphologies are observed, consistent with minimal toxicity to labeled CAR T cells (FIG. 4E, FIG. 4L) (37).

### Phenotype and Function of TAT-PFC Labeled CAR T Cells

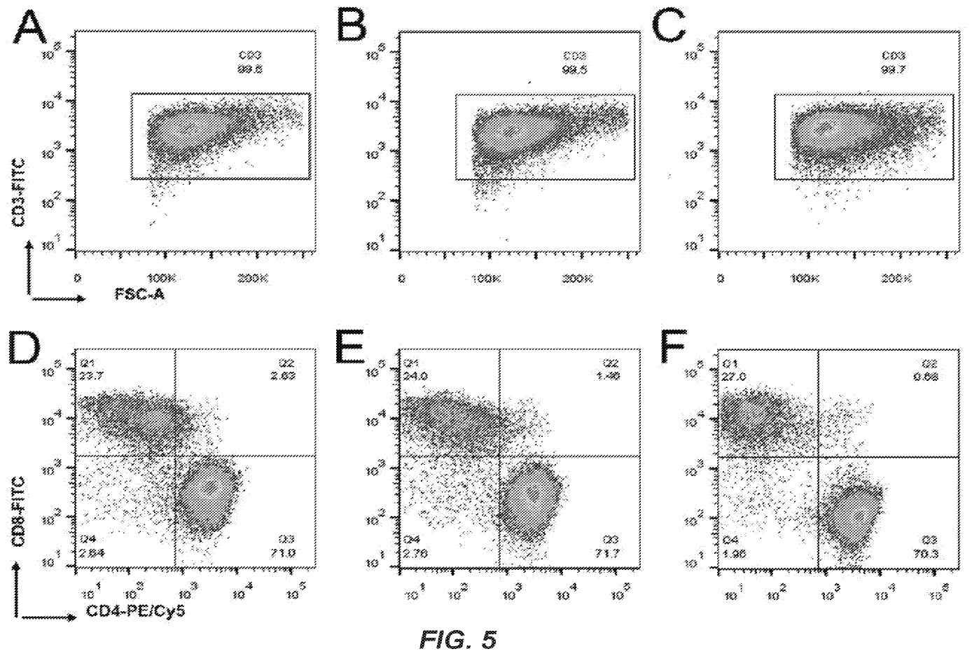

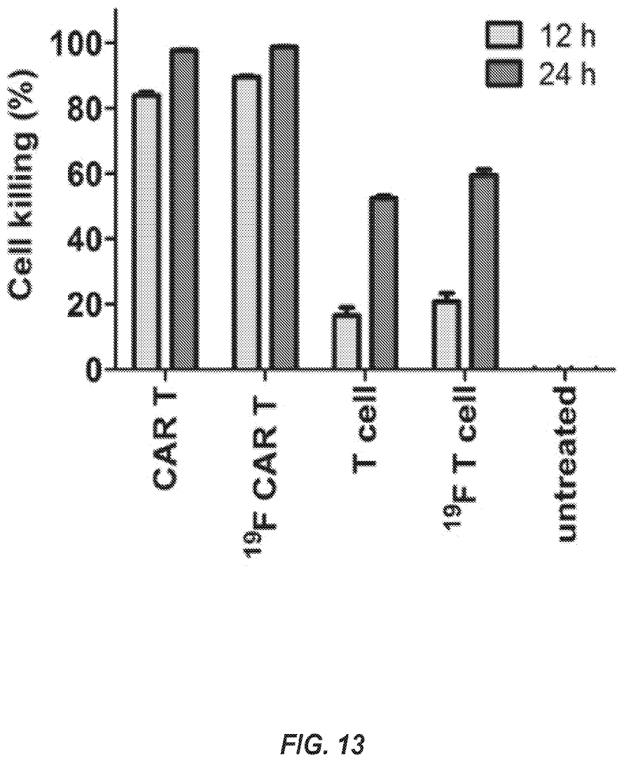

Expression of CD3, CD4 and CD8 CAR T cell surface markers is not altered by uptake of TATA-F68-PFC and TATP-F68-PFC nanoemulsions via flow cytometry (FIG. 5A-FIG. 5F). Furthermore, CAR T cell killing function against glioma cells remains intact following labeling with TATP-F68-PFC nanoemulsion (FIG. 13).

In Vivo MRI of CAR T Cells Labeled with TAT-PFC Nanoemulsion

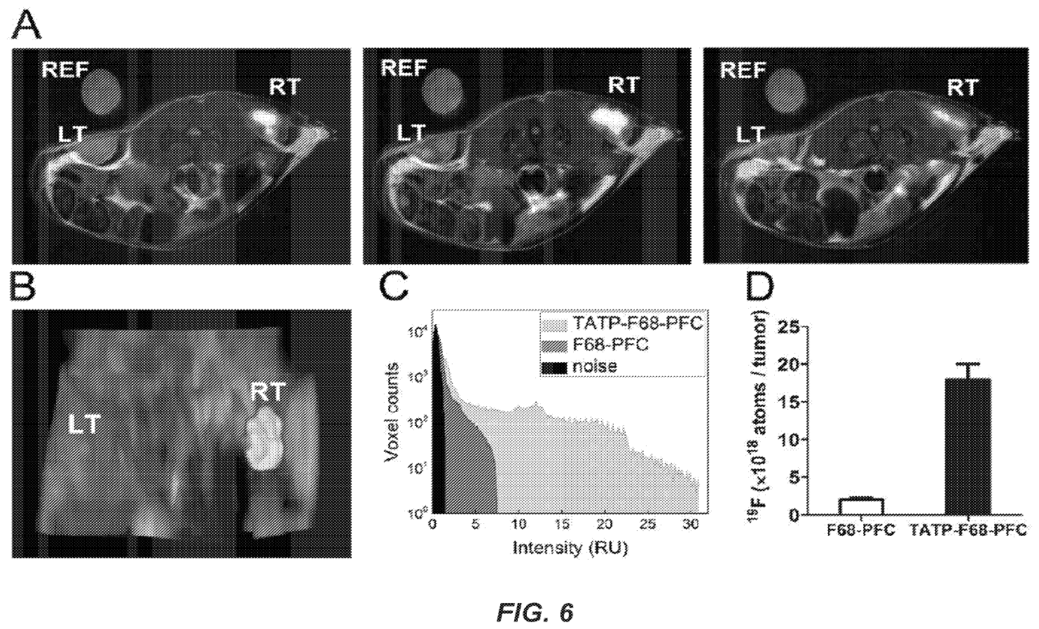

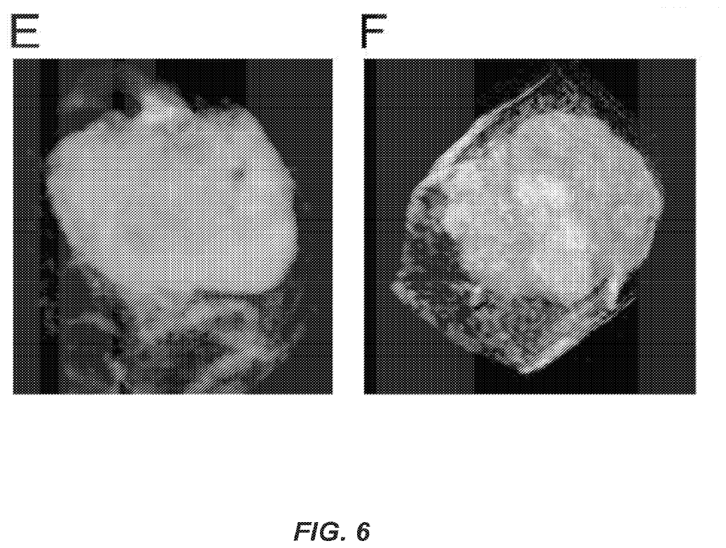

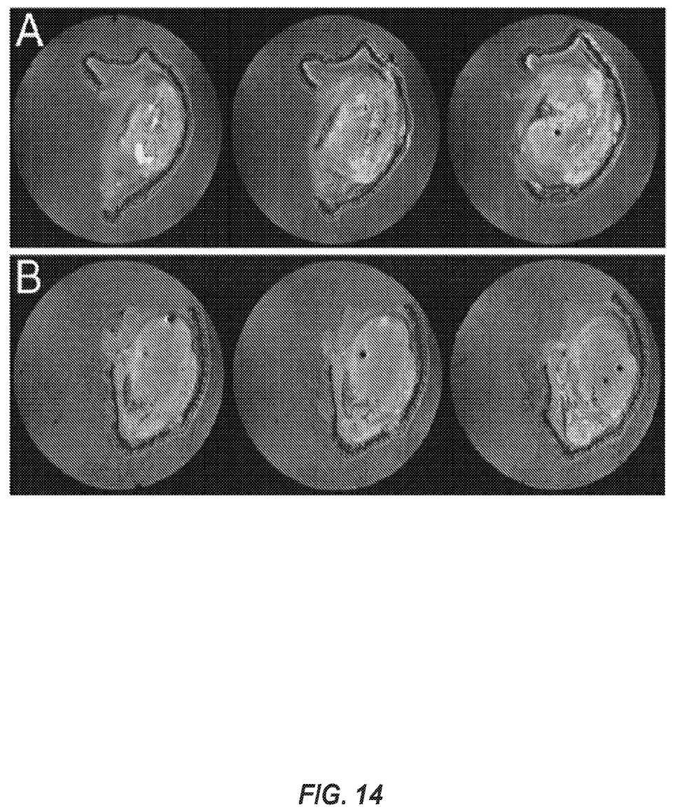

To demonstrate the utility of TAT-PFC nanoemulsion for in vivo MRI, we investigated ¹⁹F signal detection from CAR T cells implanted into a rodent model. Mice bearing bilateral flank glioma tumors were injected intra-tumorally with CAR T cells (1×10⁷ cells) labeled with either TATP-F68-PFC or F68-PFC (control) nanoemulsion. FIG. 6A, and FIG. 6B display spin-density weighted ¹⁹F images (pseudo-color) of injected cells, along with T₂-weighted ¹H images (grayscale) showing tumors in flanks. We use computational post-processing of the raw (unthresholded) image data using Voxel Tracker software to analyze the apparent ¹⁹F signal in vivo. The histogram in FIG. 6C represent the results of total signal detected in vivo. FIG. 6C displays greater number of high intensity pixels for TAT-F68-PFC-labeled CAR T cells compared to F68-PFC-labeled CAR T cells and noise. With aid of the calibrated ¹⁹F reference in the image FOV, quantification of the three-dimensional ¹⁹F images was performed; the right and left tumors display (FIG. 6D) 17.9 (±2.1)×10¹⁸ and 2.1 (±0.2)×10^{18 19}F atoms per tumor, respectively, revealing a significant (p<0.01) 8.5-fold sensitivity improvement in detection of CAR T cells labeled with TAT PFC nanoemulsion. This sensitivity increase is predicted, based on uptake analysis by ¹⁹F NMR of pelleted cells prior to injection, showing ˜8.2-fold increase in average fluorine content per cell for TATP-F68-PFC versus control nanoemulsion. Of note, the ¹⁹F/cell measured after labeling was observed to be greater for activated CAR T cells compared to the Jurkat cell line, ˜8.2-fold and ˜5-fold (FIG. 2C, FIG. 2E), respectively, when both are labeled with TATP-F68-PFC nanoemulsion. The ¹⁹F quantification results in vivo also confirm that approximately 100% of injected T cells are detected at two hours. Tumors were resected after imaging to verify intratumoral delivery of CAR T cells via extremely high-resolution ex vivo MRI (FIG. 6E, FIG. 6F, FIG. 14).

### Discussion

Fluorine-19 MRI methods have shown promise for the detection of cell therapy products post-transfer (12,14-16,18,19,38), inflammatory infiltrates (39-43) and molecular targets (44) in vivo in preclinical models. Moreover, first-generation ¹⁹F probes based on PFC nanoemulsions have been used in a pilot clinical trial (12). Overall, the utility of ¹⁹F MRI could be expanded by increasing the sensitivity of ¹⁹F probes via molecular design. Towards this goal, we aim to increase the cell adhesion and resulting endocytosis of PFC nanoemulsions by non-phagocytic cells, especially engineered lymphocytes. We synthesized three different surfactant-anchored cell penetrating TAT peptides using small molecules that either mimic the common surfactants used in emulsion formulations, namely phospholipids and poloxamers, or the fluorous environment of the nanoemulsion droplet. We compared the efficacy of poloxamer and phospholipid-based surfactants doped with TAT-conjugates and investigated nanoemulsion formation, droplet size, stability, cell uptake and viability ex vivo. Overall, nanoemulsions harboring TAT peptides led to a 4- to 8-fold cell loading improvement in T cells compared to the corresponding unmodified nanoemulsions. The droplet size, charge and initial cell safety was generally unaffected by the presence of TAT, presumably due to the low TAT to surfactant ratio in the nanoemulsions.

Phospholipid surfactants are often chosen to mimic the membranes of live cells and impart biocompatibility (45,46). Nonetheless, phospholipid-formulated nanoemulsions are prone to instability under storage conditions due to oxidation-mediated changes to the lipid (47), especially if metal ions are present, and lipid oxidation by-products may lead to cytotoxicity upon cell contact (48). Additionally, the formulation of phospholipid-based nanoemulsions requires a time consuming multi-step chemical process. For these reasons, we investigated the novel TAT conjugates for use with synthetic polymeric co-surfactants (e.g., F68) in detail.

Direct conjugation of TAT to the Pluronic F68 (TAT-F68) as a co-surfactant resulted in nanoemulsion instability (data not shown), thus instead we conjugated TAT to short linear fluorous molecules via a short aliphatic hydrocarbon linker. The nanoemulsions prepared with TATP (1a) or TATA (2a) were stable for several months at 4° C. (FIG. 8A-FIG. 8D).

As anticipated, increasing the percentage of TAT on the surface of the nanoemulsions enhanced cell uptake. However, levels >20% w/w of TAT to Pluronic surfactant resulted in increased cell-cell adhesion with formation of large cell clumps during labeling in vitro, thus a lower percent of TAT was used for further studies.

To validate internalization of the TAT nanoemulsions into CAR T cells, we prepared a comparable probe modified by the addition of a Cy5 fluorescent conjugate. The addition of this moiety did not alter the overall ¹⁹F uptake levels in T cells. Fluorescence microscopy of labeled cells shows that nanoemulsion droplets are present at the cell membrane and in the cytosol, perhaps in endosomal compartments and in various stages of internalization and cellular processing in early to late endosomes (49). PFC label is retained in the cell as long as it is viable (50), and dead cell contents are taken up by the Kupffer cells of the liver (31,51). T cell mitosis tends to dilute the label in daughter cells which may limit long-term detectability.

It has been noted that the TAT peptide can translocate nanoparticles to the nucleus (27,52), however, in our microscopy studies we did not find nuclear localization of the TAT nanoemulsion probe. We note that TATP-F68-PFC-labeled cells can optionally be washed with a diluted trypsin solution to assist with removal of surface-adhered TAT nanoemulsion prior to use in vivo. Nonetheless, presumably given sufficient time for incubation, complete endocytosis of membrane-bound TAT nanoemulsion should occur. Definitive evidence for internalized nanoemulsions in small (˜100-200 nm) to large vesicles (˜1 μm) was provided by electron microscopy. We speculate that small vesicles eventually coalesce into larger ones to minimize their hydrophobic surface area in contact with the aqueous cytosol, as cells have no mechanism for metabolizing PFCs (53,54).

While the exact mechanism of TAT- and other CPP-mediated internalization into cells remains unknown (55), the ability of CPPs to translocate molecules of various sizes from small molecules (56), proteins (57), nucleic acids and liposomes (58), many of which have been studied in vivo (59), makes their use for nanoemulsions attractive. In addition to the pre-clinical successes with TAT peptides (60-63), four compounds conjugated to TAT are currently being tested in the clinic (64) for conditions such as myocardial infarction (65), pain (66), hearing loss (66), and inflammation (67), preliminary results of phase I clinical trials indicate that TAT peptides exhibit acceptable safety profiles.

This study offers a potential strategy to assist with tracking cell populations. Our in vivo model shows that CAR T cells carrying TAT functionalized nanoemulsions give increased ¹⁹F MRI signal when injected into a tumor. Intratumoral immune cell delivery has successfully shown anti-tumor effects in mice (68) and has been considered as an approach for patients (69) as it may minimize toxicity from cytokine release syndrome upon treatment with high doses of T cells. Potential translation of this technology for human use will require additional safety testing (12). There are inherent limitations of xenograft preclinical tumor models; immunological differences, artificial tumor implants, and similar tumor size in mouse models and humans, versus vastly different doses of cytotherapy delivered to these subjects, further limit extrapolation of our model findings to human use (24).

### CONCLUSIONS

Overall, we have shown that incorporating the TAT cell penetrating peptide in PFC nanoemulsions significantly enhances cell uptake by lymphocytes and subsequently increased their detectability in vivo using ¹⁹F MRI. These same agents should be useful for tagging other weakly phagocytic cells such as stem and progenitor cells. Moreover, the peptide-PFC nanoemulsion synthesis scheme presented is generalizable for a multitude of ex vivo and in vivo targeted ¹⁹F MRI probes and offers new avenues for cellular-molecular imaging.

### REFERENCES

1. June C H, O'Connor R S, Kawalekar O U, Ghassemi S, Milone M C. CAR T cell immunotherapy for human cancer. Science 2018; 359(6382):1361-1365.
2. TownseEd M H, Shrestha G, Robison R A, O'Neill K L. The expansion of targetable biomarkers for CAR T cell therapy. J Exp Clin Cancer Res 2018; 37(1):163.
3. Bonifant C L, Jackson H J, Brentjens R J, Curran K J. Toxicity and management in CAR T-cell therapy. Mol Ther-Oncolytics 2016; 3.
4. Chang Z L, Chen Y Y. CARs: Synthetic immunoreceptors for cancer therapy and beyond. Trends Mol Med 2017; 23(5):430-450.
5. Hartmann J, Schüßler-Lenz M, Bondanza A, Buchholz C J. Clinical development of CAR T cells—challenges and opportunities in translating innovative treatment concepts. EMBO Mol Med 2017:e201607485.
6. Tarantal A F, Lee C C I, Kukis D L, Cherry S R. Radiolabeling human peripheral blood stem cells for positron emission tomography (PET) imaging in young rhesus monkeys. PloS ONE 2013; 8(10):e77148.
7. Wolfs E, Struys T, Notelaers T, Roberts S J, Sohni A, Bormans G, Van Laere K, Luyten F P, Gheysens O, Lambrichts I. 18F-FDG labeling of mesenchymal stem cells and multipotent adult progenitor cells for PET imaging: effects on ultrastructure and differentiation capacity. J Nucl Med 2013; 54(3):447-454.
8. Wang X, Rosol M, Ge S, Peterson D, McNamara G, Pollack H, Kohn D B, Nelson M D, Crooks G M. Dynamic tracking of human hematopoietic stem cell engraftment using in vivo bioluminescence imaging. Blood 2003; 102(10):3478-3482.
9. Xiong T, Zhang Z, Liu B-F, Zeng S, Chen Y, Chu J, Luo Q. In vivo optical imaging of human adenoid cystic carcinoma cell metastasis. Oral Oncol 2005; 41(7):709-715.
10. Giepmans B N G, Adams S R, Ellisman M H, Tsien R Y. The fluorescent toolbox for assessing protein location and function. Science 2006; 312(5771):217-224.
11. Ahrens E T, Bulte J W. Tracking immune cells in vivo using magnetic resonance imaging. Nat Rev Immunol 2013; 13(10):755-763.
12. Ahrens E T, Helfer B M, O'Hanlon C F, Schirda C. Clinical cell therapy imaging using a perfluorocarbon tracer and fluorine-19 MRI. Magn Reson Med 2014; 72(6):1696-1701.
13. Kircher M F, Gambhir S S, Grimm J. Noninvasive cell-tracking methods. Nat Rev Clin Oncol 2011; 8(11):677-688.
14. Bouchlaka M N, Ludwig K D, Gordon J W, Kutz M P, Bednarz B P, Fain S B, Capitini C M. (19)F-MRI for monitoring human NK cells in vivo. Oncoimmunology 2016; 5(5):e1143996.
15. Fink C, Gaudet J M, Fox M S, Bhatt S, Viswanathan S, Smith M, Chin J, Foster P J, Dekaban G A. (19)F-perfluorocarbon-labeled human peripheral blood mononuclear cells can be detected in vivo using clinical MRI parameters in a therapeutic cell setting. Sci Rep 2018; 8(1):590.
16. Janjic J M, Ahrens E T. Fluorine-containing nanoemulsions for MRI cell tracking. Wires Nanomed Nanobi 2009; 1(5):492-501.
17. Ku M C, Edes I, Bendix I, Pohlmann A, Waiczies H, Prozorovski T, Gunther M, Martin C, Pages G, Wolf S A, Kettenmann H, Uckert W, Niendorf T, Waiczies S. ERK1 as a therapeutic target for dendritic cell vaccination against high-grade gliomas. Mol Cancer Ther 2016; 15(8):1975-1987.
18. Somanchi S S, Kennis B A, Gopalakrishnan V, Lee D A, Bankson J A. In vivo (19)F-magnetic resonance imaging of adoptively transferred NK cells. Meth Mol Bio 2016; 1441:317-332.
19. Srinivas M, Turner M S, Janjic J M, Morel P A, Laidlaw D H, Ahrens E T. In vivo cytometry of antigen-specific t cells using 19F MRI. Magn Reson Med 2009; 62(3):747-753.
20. Waiczies H, Lepore S, Janitzek N, Hagen U, Seifert F, Ittermann B, Purfurst B, Pezzutto A, Paul F, Niendorf T, Waiczies S. Perfluorocarbon particle size influences magnetic resonance signal and immunological properties of dendritic cells. PLoS ONE 2011; 6(7).
21. Xander Staal O K, Mangala Srinivas. Chapter 11: In vivo 19-fluorine magnetic resonance imaging. In: Gunter Haufe FRL, editor. Fluorine in Life Sciences: Pharmaceuticals, Medicinal Diagnostics, and Agrochemicals; 2019 (Elsevier).
22. Ruiz-Cabello J, Barnett B P, Bottomley P A, Bulte J W M. Fluorine (19F) MRS and MRI in biomedicine. NMR in Biomed 2011; 24(2):114-129.
23. Ahrens E T, Zhong J. In vivo MRI cell tracking using perfluorocarbon probes and fluorine-19 detection. NMR in Biomed 2013; 26(7):860-871.
24. Chapelin F, Capitini C M, Ahrens E T. Fluorine-19 MRI for detection and quantification of immune cell therapy for cancer. J Immunother Cancer 2018; 6(1):105.
25. Lim W A, June C H. The principles of engineering immune cells to treat cancer. Cell 2017; 168(4):724-740.
26. Lindgren M, Hällbrink M, Prochiantz A, Langel Ü. Cell-penetrating peptides. Trends Pharmacol Sci 2000; 21(3):99-103.
27. Vives E, Brodin P, Lebleu B. A truncated HIV-1 Tat protein basic domain rapidly translocates through the plasma membrane and accumulates in the cell nucleus. J Biol Chem 1997; 272(25):16010-16017.
28. Janjic J M, Srinivas M, Kadayakkara D K, Ahrens E T. Self-delivering nanoemulsions for dual fluorine-19 MRI and fluorescence detection. J Am Chem Soc 2008; 130(9):2832-2841.
29. Ahrens E T, Flores R, Xu H, Morel P A. In vivo imaging platform for tracking immunotherapeutic cells. Nat Biotechnol 2005; 23(8):983-987.
30. Johnson L A, Scholler J, Ohkuri T, Kosaka A, Patel P R, McGettigan S E, Nace A K, Dentchev T, Thekkat P, Loew A. Rational development and characterization of humanized anti-EGFR variant III chimeric antigen receptor T cells for glioblastoma. Sci Transl Med 2015; 7(275):275ra222.
31. Chapelin F, Gao S, Okada H, Weber T G, Messer K, Ahrens E T. Fluorine-19 nuclear magnetic resonance of chimeric antigen receptor T cell biodistribution in murine cancer model. Sci Rep 2017; 7(1):17748.
32. Ohno M, Ohkuri T, Kosaka A, Tanahashi K, June C H, Natsume A, Okada H. Expression of miR-17-92 enhances anti-tumor activity of T-cells transduced with the anti-EGFRvIII chimeric antigen receptor in mice bearing human GBM xenografts. J Immunother Cancer 2013; 1:21.
33. Srinivas M, Morel P A, Ernst L A, Laidlaw D H, Ahrens E T. Fluorine-19 MRI for visualization and quantification of cell migration in a diabetes model. Magn Reson Med 2007; 58(4):725-734.
34. Howard M D, Hood E D, Greineder C F, Alferiev I S, Chorny M, Muzykantov V. Targeting to endothelial cells augments the protective effect of novel dual bioactive antioxidant/anti-inflammatory nanoparticles. Mol Pharm 2014; 11(7):2262-2270.
35. Hu S, Wang T, Pei X, Cai H, Chen J, Zhang X, Wan Q, Wang J. Synergistic enhancement of antitumor efficacy by PEGylated Multi-walled carbon nanotubes modified with cell-penetrating peptide TAT. Nanoscale Res Lett 2016; 11(1):452.
36. Hitchens T K, Liu L, Foley L M, Simplaceanu V, Ahrens E T, Ho C. Combining perfluorocarbon and superparamagnetic iron-oxide cell labeling for improved and expanded applications of cellular MRI. Magn Reson Med 2015; 73(1):367-375.
37. van Eeden S F, Klut Me Fau—Leal M A, Leal Ma Fau—Alexander J, Alexander J Fau—Zonis Z, Zonis Z Fau—Skippen P, Skippen P. Partial liquid ventilation with perfluorocarbon in acute lung injury: light and transmission electron microscopy studies. Am J Respir Cell Mol Biol 2000; 22(4):441-450.
38. Gonzales C, Yoshihara H A, Dilek N, Leignadier J, Irving M, Mieville P, Helm L, Michielin O, Schwitter J. In-vivo detection and tracking of T cells in various organs in a melanoma tumor model by 19F-fluorine MRS/MRI. PLoS One 2016; 11(10):e0164557.
39. Flogel U, Su S, Kreideweiss I, Ding Z P, Horning A, Witzke O, Schrader J. Early detection of transplant rejection by in vivo 19F MRI. Circulation 2009; 120(18):5817-5817.
40. Ahrens E T, Young W B, Xu H, Pusateri L K. Rapid quantification of inflammation in tissue samples using perfluorocarbon emulsion and fluorine-19 nuclear magnetic resonance. Biotechniques 2011; 50(4):229-234.
41. Khurana A, Chapelin F, Xu H, Acevedo J R, Molinolo A, Nguyen Q, Ahrens E T. Visualization of macrophage recruitment in head and neck carcinoma model using fluorine-19 magnetic resonance imaging. Magn Reson Med 2018; 79(4):1972-1980.
42. Kadayakkara D K, Ranganathan S, Young W B, Ahrens E T. Assaying macrophage activity in a murine model of inflammatory bowel disease using fluorine-19 MRI. Lab Invest 2012; 92(4):636-645.
43. Zhong J, Narsinh K, Morel P A, Xu H, Ahrens E T. In Vivo Quantification of inflammation in experimental autoimmune encephalomyelitis rats using Fluorine-19 magnetic resonance imaging reveals immune cell recruitment outside the nervous system. PLoS One 2015; 10(10):e0140238.
44. Temme S, Grapentin C, Quast C, Jacoby C, Grandoch M, Ding Z, Owenier C, Mayenfels F, Fischer J W, Schubert R, Schrader J, Flogel U. Noninvasive imaging of early venous thrombosis by 19F magnetic resonance imaging with targeted perfluorocarbon nanoemulsions. Circulation 2015; 131(16):1405-1414.
45. Grapentin C M, Friederike; Barnert, Sabine; A Süss, Regine; Schubert, Rolf; Temme, Sebastian; Jacoby, Christoph; Schrader, Juergen; Flögel, Ulrich. Optimization of perfluorocarbon nanoemulsions for molecular imaging by 19 F MRI. In: Nanomedicine. Edited by Seifalin A, de Mel A, Kalaskar D M; One Central Press, Manchester. 2014: 268-286.
46. Temme S, Baran P, Bouvain P, Grapentin C, Kramer W, Knebel B, Al-Hasani H, Moll J M, Floss D, Schrader J, Schubert R, Flogel U, Scheller J. Synthetic cargo internalization receptor system for nanoparticle tracking of individual cell populations by fluorine magnetic resonance imaging. ACS Nano 2018; 12(11):11178-11192.
47. Girotti A W. Mechanisms of lipid peroxidation. J Free Radic Biol Med 1985; 1(2):87-95.
48. Schaich K M. Metals and lipid oxidation. Contemporary issues. Lipids 1992; 27(3):209-218.
49. Fretz M, Jin J, Conibere R, Penning N A, Al-Taei S, Storm G, Futaki S, Takeuchi T, Nakase I, Jones A T. Effects of Na+/H+ exchanger inhibitors on subcellular localisation of endocytic organelles and intracellular dynamics of protein transduction domains HIV-TAT peptide and octaarginine. J Control Release 2006; 116(2):247-254.
50. Patrick M J, Janjic J M, Teng H, O'Hear M R, Brown C W, Stokum J A, Schmidt B F, Ahrens E T, Waggoner A S. Intracellular pH measurements using perfluorocarbon nanoemulsions. J Am Chem Soc 2013; 135(49):18445-18457.
51. Castro O, Nesbitt A E, Lyles D. Effect of a perfluorocarbon emulsion (Fluosol-DA) on reticuloendothelial system clearance function. Am J Hematol 1984; 16(1):15-21.
52. de la Fuente J M, Berry C C. Tat peptide as an efficient molecule to translocate gold nanoparticles into the cell nucleus. Bioconjug Chem 2005; 16(5):1176-1180.
53. Krafft M P. Fluorocarbons and fluorinated amphiphiles in drug delivery and biomedical research. Adv Drug Deliv Rev 2001; 47(2-3):209-228.
54. Krafft M P, Riess J G. Perfluorocarbons: Life sciences and biomedical uses dedicated to the memory of Professor Guy Ourisson, a true RENAISSANCE man. J Polym Sci Pol Chem 2007; 45(7):1185-1198.
55. Brooks H, Lebleu B, Vivès E. Tat peptide-mediated cellular delivery: back to basics. Adv Drug Deliv Rev 2005; 57(4):559-577.
56. Mazel M, Clair P, Rousselle C, Vidal P, Scherrmann J-M, Mathieu D, Temsamani J. Doxorubicin-peptide conjugates overcome multidrug resistance. Anti-cancer Drugs 2001; 12(2):107-116.
57. Fawell S, Seery J, Daikh Y, Moore C, Chen L L, Pepinsky B, Barsoum J. Tat-mediated delivery of heterologous proteins into cells. Proc Natl Acad Sci 1994; 91(2):664-668.
58. Torchilin V P, Levchenko T S, Rammohan R, Volodina N, Papahadjopoulos-Sternberg B, D'Souza G G M. Cell transfection in vitro and in vivo with nontoxic TAT peptide-liposome-DNA complexes. Proc Natl Acad Sci 2003; 100(4):1972-1977.
59. Foged C, Nielsen H M. Cell-penetrating peptides for drug delivery across membrane barriers. Expert Opin Drug Deliv 2008; 5(1):105-117.
60. Boisguerin P, Redt-Clouet C, Franck-Miclo A, Licheheb S, Nargeot J, Barrère-Lemaire S, Lebleu B. Systemic delivery of BH4 anti-apoptotic peptide using CPPs prevents cardiac ischemia-reperfusion injuries in vivo. J Control Release 2011; 156(2):146-153.
61. Borsello T, Clarke P G H, Hirt L, Vercelli A, Repici M, Schorderet D F, Bogousslavsky J, Bonny C. A peptide inhibitor of c-Jun N-terminal kinase protects against excitotoxicity and cerebral ischemia. Nat Med 2003; 9(9):1180.
62. Michiue H, Eguchi A, Scadeng M, Dowdy S F. Induction of in vivo synthetic lethal RNAi responses to treat glioblastoma. Cancer Biol Ther 2009; 8(23):2304-2311.
63. Yang D, Sun Y-Y, Lin X, Baumann J M, Dunn R S, Lindquist D M, Kuan C-Y. Intranasal delivery of cell-penetrating anti-NF-κB peptides (Tat-NBD) alleviates infection-sensitized hypoxic-ischemic brain injury. Exp Neurol 2013; 247:447-455.
64. Direct Inhibition of delta-Protein Kinase CEtLTISiAMII, Bates E, Bode C, Costa M, Gibson C M, Granger C, Green C, Grimes K, Harrington R, Huber K, Kleiman N, Mochly-Rosen D, Roe M, Sadowski Z, Solomon S, Widimsky P. Intracoronary KAI-9803 as an adjunct to primary percutaneous coronary intervention for acute ST-segment elevation myocardial infarction. Circulation 2008; 117(7):886-896.
65. Cousins M J, Pickthorn K, Huang S, Critchley L, Bell G. The safety and efficacy of KAI-1678—an inhibitor of epsilon protein kinase C (epsilon PKC)-versus lidocaine and placebo for the treatment of postherpetic neuralgia: a crossover study design. Pain Med 2013; 14(4):533-540.
66. Suckfuell M, Lisowska G, Domka W, Kabacinska A, Morawski K, Bodlaj R, Klimak P, Kostrica R, Meyer T. Efficacy and safety of AM-111 in the treatment of acute sensorineural hearing loss: a double-blind, randomized, placebo-controlled phase II study. Otol Neurotol 2014; 35(8):1317-1326.
67. Deloche C, Lopez-Lazaro L, Mouz S, Perino J, Abadie C, Combette J M. XG-102 administered to healthy male volunteers as a single intravenous infusion: a randomized, double-blind, placebo-controlled, dose-escalating study. Pharmacol Res Perspect 2014; 2(1):e00020.
68. Tatsumi T, Huang J, Gooding W E, Gambotto A, Robbins P D, Vujanovic N L, Alber S M, Watkins S C, Okada H, Storkus W J. Intratumoral delivery of dendritic cells engineered to secrete both interleukin (IL)-12 and IL-18 effectively treats local and distant disease in association with broadly reactive Tcl-type immunity. Cancer Res 2003; 63(19):6378-6386.
69. Ngwa W, Irabor O C, Schoenfeld J D, Hesser J, Demaria S, Formenti S C. Using immunotherapy to boost the abscopal effect. Nat Rev Cancer 2018; 18(5):313-322.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A nanoemulsion formulation comprising a surfactant, a perfluorocarbon, and a hydrophilic anchor, wherein said perfluorocarbon is conjugated to said hydrophilic anchor via a linker.

2. The nanoemulsion formulation of claim 1, wherein said hydrophilic anchor interacts with the one or more cells.

3. The nanoemulsion formulation of claim 1 or 2, wherein said hydrophilic anchor is at an amount of at least 2% (w/w) of said hydrophilic anchor to said surfactant.

4. The nanoemulsion formulation of any one of claims 1 to 3, wherein the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

5. The nanoemulsion formulation of any one of claims 1 to 4, wherein said perfluorocarbon comprises perfluoropolyether (PFPE) or perfluoro-15-crown-5-ether (PFCE).

6. The nanoemulsion formulation of any one of claims 1 to 5, wherein said linker is an aliphatic hydrocarbon linker.

7. The nanoemulsion formulation of any one of claims 1 to 6, wherein the surfactant comprises a block copolymer of polyethylene and polypropylene glycol.

8. The nanoemulsion formulation of any one of claims 1 to 7, wherein said nanoemulsion further comprises a detectable moiety.

9. The nanoemulsion formulation of claim 8, wherein said detectable moiety is attached to said perfluorocarbon.

10. The nanoemulsion formulation of claim 8 or 9, wherein said detectable moiety is a fluorescent moiety.

11. A nanoemulsion formulation comprising a poloxamer surfactant, a perfluorocarbon, and a TAT peptide of HIV, wherein said perfluorocarbon is conjugated to said hydrophilic anchor via a linker.

12. A nanoemulsion formulation comprising a poloxamer surfactant, a perfluorocarbon, and a TAT peptide of HIV, wherein said perfluorocarbon is conjugated to said hydrophilic anchor via a linker and said TAT peptide is an amount of at least 2% (w/w) of TAT peptide to surfactant.

13. A nanoemulsion formulation comprising a poloxamer surfactant, perfluoropolyether (PFPE), and a TAT peptide of HIV, wherein said PFPE is conjugated to said hydrophilic anchor via a linker.

14. A nanoemulsion formulation comprising a poloxamer surfactant, perfluoro-15-crown-5-ether (PFCE), and a TAT peptide of HIV, wherein said PFCE is conjugated to said hydrophilic anchor via a linker.

15. A nanoemulsion formulation comprising a poloxamer surfactant, perfluoropolyether (PFPE), and a TAT peptide of HIV, wherein said PFPE is conjugated to said hydrophilic anchor via a linker and said TAT peptide is an amount of at least 2% (w/w) of TAT peptide to surfactant.

16. A nanoemulsion formulation comprising a poloxamer surfactant, perfluoro-15-crown-5-ether (PFCE), and a TAT peptide of HIV, wherein said PFCE is conjugated to said hydrophilic anchor via a linker and said TAT peptide is an amount of at least 2% (w/w) of TAT peptide to surfactant.

17. The nanoemulsion formulation of any one of claims 11 to 16, wherein said nanoemulsion further comprises a detectable moiety.

18. The nanoemulsion formulation of claim 17, wherein said detectable moiety is attached to said perfluorocarbon, PFPE, or PFCE.

19. The nanoemulsion formulation of claim 17 or 18, wherein said detectable moiety is a fluorescent moiety.

20. A non-invasive imaging method comprising:
a) administering to a subject a cellular labelling composition comprising (i) a compound comprising fluorine-19 (¹⁹F) and (ii) a nanoemulsion comprising a surfactant, a perfluorocarbon, and a hydrophilic anchor,
wherein said perfluorocarbon is conjugated to said hydrophilic anchor via a linker, said hydrophilic anchor interacts with the one or more cells, and wherein said composition associates with one or more cells; and
b) detecting said association using an imaging modality, wherein said association can include cellular binding and/or cellular uptake.

21. The method of claim 20, wherein said imaging modality is selected from the group consisting of magnetic resonance imaging (MRI), positron emission tomography (PET), single-photon emission coherent tomography (SPECT), ultrasonography (US), and computed tomography (CT).

22. The method of claim 20 or 21, wherein said compound comprising fluorine-19 (¹⁹F) comprises a perfluorinated compound.

23. The method of any one of claims 20 to 22, wherein said hydrophilic anchor is at an amount of at least 2% (w/w) of said hydrophilic anchor to said surfactant.

24. The method of any one of claims 20 to 23, wherein the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

25. The method of claim 24, wherein said cell penetrating peptide comprises a transactivating transcription sequence.

26. The method of claim 25, said cell penetrating peptide comprises a transactivating transcription sequence of the human immunodeficiency virus.

27. The method of any one of claims 20 to 26, wherein said perfluorocarbon comprises perfluoropolyether (PFPE) or perfluoro-15-crown-5-ether (PFCE).

28. The method of any one of claims 20 to 27, wherein said linker is an aliphatic hydrocarbon linker.

29. The method of any one of claims 20 to 28, wherein said surfactant comprises a block copolymer of polyethylene and polypropylene glycol.

30. The method of any one of claims 20 to 29, wherein said nanoemulsion further comprises a detectable moiety.

31. The method of claim 30, wherein said detectable moiety is attached to said perfluorocarbon.

32. The method of claim 30 or 31, wherein said detectable moiety is a fluorescent moiety.

33. The method of any one of claims 20 to 32, wherein said composition allows tracking cells by MRI, wherein said method comprises detecting the cells associated with at least one component of the composition comprising fluorine-19 (¹⁹F).

34. The method of any one of claims 20 to 33, wherein said one or more cells are immune cells that accumulate at tissue sites as part of an inflammatory response.

35. The method of any one of claims 20 to 34, wherein said method is a diagnostic detection method.

36. The method of any one of claims 20 to 33, wherein said one or more cells are engineered immune cells that are administered to said subject to treat a disease or condition.

37. The method of any one of claims 20 to 36, wherein said method is cytotherapy.

38. The method of any one of claims 20 to 37, wherein said one or more cells are endogenous cells of said subject.

39. The method of any one of claims 20 to 38, wherein said one or more cells are selected from the group consisting of T cells, B cells, macrophages, natural killer (NK) cells, dendritic cells (DCs), stem cells, progenitor cells, and cancer cells.

40. The method of any one of claims 20 to 39, wherein said one or more cells comprise engineered cells.

41. The method of claim 40, wherein said one or more cells are engineered chimeric antigen receptor (CAR) T cells that are administered to a subject to treat cancer.

42. The method of any one of claims 20 to 41, wherein said compound comprising fluorine-19 (¹⁹F) is a dual-mode agent and is capable of being detected by more than one imaging modality.

43. The method of any one of claims 20 to 42, wherein said compound comprising fluorine-19 (¹⁹F) is a dual-mode agent and is capable of being detected by two or more imaging modalities.

44. An in vivo imaging method comprising:
a) ex vivo labeling cells with a cellular labelling composition comprising (i) a compound comprising fluorine-19 (¹⁹F) and (ii) a nanoemulsion comprising a surfactant, a perfluorocarbon, and a hydrophilic anchor, wherein said perfluorocarbon is conjugated to said hydrophilic anchor via a linker under such conditions that said composition is internalized by the cells;
b) administering the labeled cells to a subject;
c) detecting said labeled cells in said subject using an imaging modality; and
d) assaying for the degree of cell accumulation in one or more tissues in said subject.

45. The method of claim 44, wherein said assaying comprises quantitating an average total intracellular probe mass at one or more sites of accumulation of said labeled cells.

46. The method of claim 44 or 45, wherein said cells are autologous cells.

47. The method of claim 44 or 45, wherein said cells are allogeneic cells.

48. The method of any one of claims 44 to 47, wherein said imaging modality is selected from the group consisting of magnetic resonance imaging (MRI), positron emission tomography (PET), single-photon emission coherent tomography (SPECT), ultrasonography (US), and computed tomography (CT).

49. The method of claim 48, wherein said imaging modality is magnetic resonance imaging (MRI).

50. The method of any one of claims 44 to 49, wherein said cells are selected from the group consisting of T cells, B cells, macrophages, natural killer (NK) cells, dendritic cells (DCs), stem cells, progenitor cells, and cancer cells.

51. The method of any one of claims 44 to 50, wherein said cells are engineered cells.

52. The method of any one of claims 44 to 51, wherein said compound comprising fluorine-19 (¹⁹F) comprises a perfluorinated compound.

53. The method of any one of claims 44 to 52, wherein said hydrophilic anchor is at an amount of at least 2% (w/w) of said hydrophilic anchor to said surfactant.

54. The method of any one of claims 44 to 53, wherein said hydrophilic anchor interacts with the one or more cells.

55. The method of any one of claims 44 to 54, wherein the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

56. The method of any one of claims 44 to 55, wherein said perfluorocarbon comprises perfluoropolyether (PFPE) or perfluoro-15-crown-5-ether (PFCE).

57. The method of any one of claims 44 to 56, wherein said linker is an aliphatic hydrocarbon linker.

58. The method of any one of claims 44 to 57, wherein the surfactant comprises a block copolymer of polyethylene and polypropylene glycol.

59. The method of any one of claims 44 to 58, wherein said nanoemulsion further comprises a detectable moiety.

60. The method of claim 59, wherein said detectable moiety is attached to said perfluorocarbon.

61. The method of claim 59 or 60, wherein said detectable moiety is a fluorescent moiety.

62. A pharmaceutical and/or diagnostic composition comprising a compound comprising fluorine-19 (¹⁹F) and a nanoemulsion formulation comprising a surfactant, a perfluorocarbon, and a hydrophilic anchor, wherein said perfluorocarbon is conjugated to said hydrophilic anchor via a linker, wherein said composition associates with one or more cells, and wherein said association is capable of being detected using an imaging modality.

63. The pharmaceutical and/or diagnostic composition of claim 62, wherein said compound comprising fluorine-19 (¹⁹F) comprises a perfluorinated compound.

64. The pharmaceutical and/or diagnostic composition of claim 62 or 63, wherein said hydrophilic anchor interacts with the one or more cells.

65. The pharmaceutical and/or diagnostic composition of any one of claims 62 to 64, wherein the hydrophilic anchor is at an amount of at least 2% (w/w) of said hydrophilic anchor to said surfactant.

66. The pharmaceutical and/or diagnostic composition of any one of claims 62 to 65, wherein the hydrophilic anchor is selected from the group consisting of a cell penetrating peptide, a peptide rich in lysine, arginine and histidines, a peptide or small molecule ligand that binds to a specific cell surface receptor or target cell type, an oligonucleotide, an antibody, a nanobody, an aptamer, and a moiety useful for enhanced endocytosis or cell adhesion.

67. The pharmaceutical and/or diagnostic composition of any one of claims 62 to 66, wherein said perfluorocarbon comprises perfluoropolyether (PFPE) or perfluoro-15-crown-5-ether (PFCE).

68. The pharmaceutical and/or diagnostic composition of any one of claims 62 to 67, wherein said linker is an aliphatic hydrocarbon linker.

69. The pharmaceutical and/or diagnostic composition of any one of claims 62 to 68, wherein the surfactant comprises a block copolymer of polyethylene and polypropylene glycol.

70. The pharmaceutical and/or diagnostic composition of any one of claims 62 to 69, wherein said nanoemulsion further comprises a detectable moiety.

71. The pharmaceutical and/or diagnostic composition of claim 70, wherein said detectable moiety is attached to said perfluorocarbon.

72. The pharmaceutical and/or diagnostic composition of claim 70 or 71, wherein said detectable moiety is a fluorescent moiety.

73. The pharmaceutical and/or diagnostic composition of any one of claims 62 to 72, wherein said composition comprises at least two compounds comprising fluorine-19 (¹⁹F), wherein the at least two compounds provide at least two distinct signatures when detected using an imaging modality capable of individual detection.

74. The pharmaceutical and/or diagnostic composition of any one of claims 62 to 73, wherein said imaging modality is selected from the group consisting of magnetic resonance imaging (MRI), positron emission tomography (PET), single-photon emission coherent tomography (SPECT), ultrasonography (US), and computed tomography (CT).

75. The pharmaceutical and/or diagnostic composition of claim 73 or 74, wherein said distinct signatures correspond to multiple cell types, the same cell type at different time points, or multiple molecular epitopes within a subject.

76. The pharmaceutical and/or diagnostic composition of any one of claims 62 to 75, wherein said compound comprising fluorine-19 (¹⁹F) is a theranostic agent.

77. The pharmaceutical and/or diagnostic composition of claim 76, wherein said theranostic agent functions as both a therapeutic agent and an imaging probe.

78. The pharmaceutical and/or diagnostic composition of claim 77, wherein said theranostic agent allows for visualizing the accurate delivery and dose of the therapy within the subject.
